(19) United States
(12) Patent Application Publication
(10) Pub. No.: US 2004/0170995 A1
(43) Pub. Date: Sep. 2, 2004

(54) Isolated nucleic acid molecules encoding a novel human signal transducing kinase-mapkap-2; encoded proteins, cells transformed therewith and uses thereof

(76) Inventors: Phillip Lograsso, Carlsbad, CA (US); Julio Hawkins, Summit, NJ (US); Jean Marie Lisnock, Middletown, NJ (US)

Correspondence Address: MERCK AND CO INC, P O BOX 2000, RAHWAY, NJ, 070650907

(21) Appl. No.: 10/469,221

(22) PCT Filed: Feb. 25, 2002
(86) PCT No.: PCT/US02/05670

(30) Foreign Application Priority Data
Feb. 28, 2001 (US) 60272260

(51) Int. Cl. C12Q 1/68; C07H 21/04; C12N 9/12

(52) U.S. Cl. 435/6; 435/69.1; 435/194; 435/320.1; 435/325; 536/23.2

(57) ABSTRACT

Disclosed herein are two newly identified enzymes—mitogen-activated protein kinase-activated protein kinase-2, each comprising a sequence of nucleotides as set forth in SEQ ID NOs: 1 and 3. Each of the herein disclosed enzymes is a serine/threonine signal transduction kinase that is phosphorylated and activated by Erks and p38 MAPK in vitro. Also disclosed, inter alia, are cells containing the recombinant nucleic acid molecules; antisense constructs thereto; antibodies specific for each of the disclosed proteins; methods for using the novel nucleic acid molecules and their gene products including kits containing the same.

### CROSS-REFERENCE TO RELATED APPLICATIONS

Not Applicable.

### STATEMENT REGARDING FEDERALLY-SPONSORED R&D

Not Applicable.

### REFERENCE TO MICROFICHE APPENDIX

Not Applicable.

### FIELD OF THE INVENTION

The present invention relates to newly identified nucleic acid molecules encoding intracellular signal-transduction serine/threonine kinases, polypeptides encoded by the polynucleotides, and antibodies raised against the novel polypeptides. The invention further provides for the use of the novel polynucleotides and polypeptides, e.g., methods to regulate signal transduction in a cell and methods for utilizing the herein disclosed MAPKAP-2 kinase in the design of protocols for the treatment of MAPKAP-2 mediated disorders. Also provided are therapeutic compositions and their use to treat mammals having medical disorders manifested by a dysfunctional signal transducing pathway of which the disclosed MAPKAP-2 kinase is an integral member or a dysfunctional MAPKAP-2 kinase.

### BACKGROUND OF THE INVENTION

Regulation of cell growth is mediated by a complex array of signaling pathways precisely coordinated by different families of cell surface receptors that serve to “sense” the cell environment and supply the cell with an input signal about any changes in the environment. These signaling pathways regulate all the critical phases of cell growth which lead to changes in protein synthesis, secretion, metabolism and gene expression. These signals take the form of growth factors, hormones, cytokines, and peptides, which bind to and activate specific receptor molecules located on cell surface membranes. The activated receptors, in turn, trigger intracellular signal transduction pathways ultimately culminating in a wide range of cellular responses affecting gene expression, protein secretion, cell cycle progression, and cell differentiation. Signaling pathways within cells are generally formed by chains of intercommunicating proteins, wherein each protein component integrates signals from upstream activators and passes them to various downstream targets or effector proteins.

Reversible protein phosphorylation is a central feature of signal transduction. Many cellular processes are known to be modulated in some manner by phosphorylation/dephosphorylation mechanisms. In fact, a number of protein kinase “cascades” have been recognized, in which a series of protein kinases phosphorylate and regulate one another in a sequential fashion ultimately leading to differential phosphorylation of key regulatory molecules such as metabolic enzymes, transcription factors, cytoskeleton proteins, cell-cycle regulators, translation control proteins, ion channels and other protein kinases. Thus, protein kinases and phosphatases play a major role in cell regulation.

It is estimated that more than 1000 of the 10,000 proteins active in a typical mammalian cell are phosphorylated. The high-energy phosphate which drives activation is generally transferred from adenosine triphosphate molecules (ATP) to a particular protein by protein kinases and removed from that protein by protein phosphatases. Phosphorylation occurs in response to extracellular signals (hormones, neurotransmitters, growth and differentiation factors, etc.), cell cycle checkpoints, and environmental or nutritional stresses, and is roughly analogous to turning on a molecular switch. When the switch goes on, the appropriate protein kinase activates a metabolic enzyme, regulatory protein, receptor, cytoskeletal protein, ion channel or pump, or transcription factor.

Protein kinases are enzymes that phosphorylate other proteins and/or themselves (autophosphorylation). With regard to substrates, protein kinases involved in signal transduction in eukaryotic cells can be divided into three major groups based upon their substrate utilization: protein-tyrosine specific kinases (which phosphorylate substrates on tyrosine residues), protein-serine/threonine specific kinases (which phosphorylate substrates on serine and/or threonine residues) and dual-specificity kinases (which phosphorylate substrates on tyrosine, serine and/or threonine residues). Well over a hundred protein kinases have been identified to date. For reviews on protein kinases, see Kemp, B. E. (ed.), Peptides and Protein Phosphorylation, CRC Press Inc. (1990) and Hanks et al., Science 241:42-52 (1988).

Given their key role in cellular regulation, it is not surprising that defects in protein kinases have been implicated in many disease states and conditions. For example, the over-expression of cellular tyrosine kinases such as the EGF or PDGF receptors, or the mutation of tyrosine kinases to produce constitutively active forms (oncogenes) occurs in many cancer cells. See Drucker et al., Nature Medicine 2: 561-56 (1996). Protein tyrosine kinases are also implicated in inflammatory signals. Defective Thr/Ser kinase genes have been demonstrated to be implicated in several diseases such as myotonic dystrophy as well as cancer, and Alzheimer's disease. Sanpei et al. Biochem. Biophys. Res. Commun. 212: 341-6 (1995); Sperber et al. Neurosci. Lett. 197: 149-153 (1995); Grammas et al. Neurobiology of Aging 16: 563-569 (1995); Govoni et al. Ann. N.Y. Acad. Sci. 777: 332-337 (1996). More, kinases have also been implicated in angiotensin II and hematopoietic cytokine receptor signal transduction. B. Berk et al., Circ. Res., 80:5, pp. 607-16 (1997); R. Mufson, FASEB J., 11:1 pp. 37-44 (1997).

Mitogen-activated protein kinases (MAP kinases/MAPK), a family of serine/threonine protein kinases, have been recently demonstrated to play a central role in mediating the intracellular actions of a variety of extracellular agonists that include growth factors, mitogens and differentiating agents whose receptors are protein tyrosine kinase (Cobb et al., 1991; Sturgill and Wu, 1991).

Over the past few years, data suggest that MAP kinase facilitates signal transduction through both protein kinases and protein phosphatases and is responsible for triggering biological effects across a wide variety of pathophysiological conditions. These include conditions manifested by dysfunctional leukocytes; T-lymphocytes; acute and chronic inflammatory diseases such as rheumatoid arthritis, Crohn's disease, inflammatory bowl disease; osteoarthritis; atherosclerosis; auto-immune disorders; asthma and allergic response.

Nearly all cell surface receptors use one or more of the MAP protein kinase “cascades” during signal transduction. Signals from diverse receptors, including receptor protein tyrosine kinases, nonreceptor protein tyrosine kinases, cytokine receptors, and heterotrimeric G protein-coupled receptors have all been report to result in activation of the MAPKs.

The MAP kinase cascade, is activated as an early event in the response of cells to a wide variety of stimuli. The mechanism of activation of MAP kinases has been intensively investigated and reveals a conserved signaling cascade initiated by ligand induced activation of receptor tyrosine kinases which leads to a sequential activation of a series of protein kinases. The variety of signals that conscript the MAP kinase pathway demonstrates that this cascade serves a myriad of purposes, and the consequences of its activation will depend on cellular context.

MAP kinases are activated by phosphorylation at a dual phosphorylation motif with the sequence Thr-X-Tyr by mitogen-activated protein kinase kinases (MAPKKs) which is itself ‘dual specificity’ enzyme (Ahn et al., 1991; Gomez and Cohen, 1991; Nakielny et al., 1992a,b). The dual-specificity kinase is capable of phosphorylating both tyrosine and serine/threonine residues in proteins. Activated MAPK undergoes a translocation to the nucleus where it can directly phosphorylate and activate a variety of transcription factors including c-Myc, C/EBP.beta., p62.sup.TCF/Elk-1, ATF-2 and c-Jun. Grunicke, Hans H., Signal Transduction Mechanisms in Cancer, Springer-Verlag (1995).

The mammalian MAP kinases include the extracellular signal regulated kinases (ERKs) the c-Jun N-terminal kinases (JNKs) and the CSBP/p38/RK/Mpk2 kinases. (Cobb et al.). These subgroups are distinguished by the sequence of the tripeptide dual phosphorylation motif that is required for MAP kinase activation: Thr-Glu-Tyr (ERK), Thr-Pro-Tyr (JNK), and Thr-Gly-Tyr (p38). The best-characterized pathway leads to the activation of the extracellular-signal-regulated kinase (ERK). Less well understood are the signal transduction pathways leading to the activation of the cJun N-terminal kinase (JNK) and the p38 MAPK (for reviews, see Davis, Trends Biochem. Sci. 19:470-473 (1994). Each pathway contains a MAP kinase module, consisting of a MAP kinase or ERK, a MAP/ERK kinase (MEK), and a MEK kinase (MEKK).

All known signaling pathways are believed to use the two dual specificity protein kinases MEK1 and MEK2 to phosphorylate and activate MAP kinase. Substrates of the MAP kinases include other protein kinases and several transcription factors, which are activated by phosphorylation to induce gene expression.

JNK and p38 kinases are activated in response to the pro-inflammatory cytokines TNF-α and interleukin-1, and by cellular stress such as heat shock, ultraviolet radiation, lipopolysaccharides and inhibitors of protein synthesis. See B. Derijard et al., Cell, 76, pp. 1025-37 (1994); L. Shapiro et al., Proc. Natl. Acad. Sci. U.S.A., 92, pp. 12230-4 (1995). Activation of the MAPK kinase, particularly the p38 pathway results in phosphorylation of transcription and initiation factors, and affects cell division, apoptosis, invasiveness of cultured cells and the inflammatory response.

p38 MAP kinase activates many protein kinases such as the MAP-kinase-activated protein kinases MAPKAP-2 and MAPKAP-3, the MAP-kinase-interacting kinases MNK1 and MNK2, and the p38-regulated/activated protein kinases PRAK and MSK1. Moreover, transcription factors such as ATF2 (activating transcription factor 2), CHOP/GADD153 and MEF2 (myocyte enhancer factor 2) are phosphorylated in parallel. Recently, the nucleus has been shown to be a target for the signal transduction of p38 MAP kinases.

In sharp contrast, ERK kinases are activated by mitogens and growth factors. See D. Bokemeyer et al., Kidney Int., 49, pp. 1187-98 (1996). The ERK cascade starts with one or more Raf family kinases, which phosphorylate and activate MAP kinase kinase 1 (MKK1) and MKK2, permitting the MKKs to phosphorylate and activate the MAP kinases extracellular signal-regulated kinase 1 (Erk1) and Erk2 (Davis, 1993; Marshall, 1995). Erks then phosphorylate both cytoplasmic and nuclear substrates, including the epidermal growth factor receptor, cytosolic phospholipase A2, ribosomal S6 kinase II (Rsk). Elk-1 (a ternary complex factor), c-Jun and c-Myc (Sturgill et al., 1988; Pulverer et al., 1991; and Gille et al., 1995). A net result of activating the ERK cascade is induction of immediate early gene transcription leading to cell proliferation or differentiation. Upon activation, MAP kinases have been reported to translocate into the nucleus and phosphorylate transcription factors.

Among the substrates for MAP kinases are S6 kinases—rsk1 and rsk2 (p90rsk) and MAPKAP kinase-2 (MAPKAP-2), which is an acronym for mitogen-activated protein kinase-activated protein kinase-2.

MAPKAP-2 was originally identified in skeletal muscle as an enzyme that is activated by the p42 and p44 isoforms of mitogen-activated protein MAP kinase (MAPK) (Stokoe et al., 1992a) and distinguished from the RSK family of ribosomal protein S6 kinases (MAPKAP kinases-1), which are also activated by p42/p44MAPK, by its substrate specificity, insensitivity to the inhibitor H7, and amino acid sequence (Stokoe et al., 1992a, 1993). The catalytic domain of MAPKAP kinase-2 is most similar (35%40% identity) to a branch of the protein kinase phylogenetic tree that includes several calmodulin-dependent protein kinases and the C-terminal kinase domain of MAPKAP kinase-2 (Stokoe et al., 1993, Engel et al., 1993). It is preceded by a proline-rich N-terminal domain, while two different C-terminal sequences have been reported that may be generated by alternative splicing of a common precursor mRNA. One of these isoforms contains a putative nuclear localization signal KK(X)₁₀KRRKK (Stokoe et al., 1993; Zu et al., 1994). The MAPKAP-2 mRNA transcript is present at a similar concentration in every mammalian tissue examined (Stokoe et al., 1993; Zu et al., 1994).

Substrates of MAPKAP-2 include glycogen synthase; small heat shock proteins Hsp-25 and Hsp-27, ATF2 and CREB (Stokoe et al., 1992b; Freshney et al., 1994; Tan et al., 1996). The best characterized substrate for MAPKAP-2 is Hsp27.

MAPKAP-2 becomes activated when cells are stimulated with interleukin-1 or tumour necrosis factor or exposed to cellular stresses (Rouse et al., 1994). MAPKAP-2 is also phosphorylated and activated by Erks in vitro. In cells, however, it is predominantly regulated by p38 MAPK. In the inflammatory response, TNF-α and IL-β activate p38, which triggers production of more TNF and IL-1, which, in turn, amplifies the inflammatory response. This process is thought to play a role in septic shock and formation of the athersclerotic plaque.

In view of the recent literature, it is generally accepted that the p38 kinase and MAPKAP-2 kinase pathway are central components in the response of cells to chemical, mechanical and proinflammatory assaults.

Clear evidence has been shown, for instance, that ERK, JNK and p38 pathways are strongly linked to IL-2 production. P38 activation is clearly established to be required for full T-lymphocyte activation leading to IL-2 gene transcription and T-cell proliferation. Interruption of the signaling process by selective kinase inhibition is therefore expected to reduce IL-2 production and T-lymphocyte proliferation which would be therapeutically beneficial in chronic inflammatory diseases including but not limited to rheumatoid arthritis, Crohn's disease and inflammatory bowl disease.

The MAPKAP-2 kinase described herein (SEQ ID NO:2) is believed to transduce cellular response to stress related stimuli leading to the activation of proinflammatory gene products. The deleterious effects of the mediators of inflammation, including cytokines, opens new avenues for the development of original anti-inflammatory therapies. As MAPKs and MAPKAP-2 play a central role in signaling events which mediate cellular response to stress, their inactivation or antagonization is key to the attenuation of the response.

MAPKs including MAPKAP-2 are thus expected to play key roles in the pathology of autoimmune diseases including but not limited to rheumatoid arthritis (RA), Crohn's disease, inflammatory bowl disease, osteoarthritis (OA), and multiple sclerosis (MS).

Accordingly, the novel human serine/threonine signal-transduction kinase described herein (SEQ ID NO:2), and nucleic acid sequence coding therefor, e.g. SEQ ID NO:1 has significant potential as a specific targets that can be exploited diagnostically and therapeutically for the control of dysfunctional leukocytes, including but not limited to dysfunctional T-lymphocytes, and in the treatment of chronic inflammation including rheumatoid arthritis (RA), Crohn's disease, inflammatory bowl disease, osteoarthritis (OA) and autoimmune disease, and in the study, diagnosis, and treatment of acute and chronic inflammation as well as other diseases manifested by dysfunctional native MAPKAP-2 kinase.

Indeed, potential diagnostic and therapeutic applications are readily apparent for modulators of the human MAPKAP-2 signal-transduction serine/threonine kinase described herein. Areas which are common to disease particularly in need of therapeutic intervention include but are not limited to pathophysiological disorders manifested by dysfunctional leukocytes, T-cell activation, acute and chronic inflammatory disease such as rheumatoid arthritis, Crohn's disease and inflammatory bowl disease, auto-immune disorders, osteoarthritis, transplant rejection, macrophage regulation, atherosclerosis, fibroblasts regulation, pathological fibrosis, asthma, allergic response, atheroma, osteoarthritis, sepsis, neurodegeneration, and related disorders.

Compounds which modulate or inactivate specific signal transduction polypeptide(s) integral to specific cytosolic pathways generally will have significant potential for the ability to modulate or attenuate downstream physiological responses. Accordingly, the ability to screen for compounds which modulate the activity of native or recombinant human MAPKAP-2 kinase such as the one described herein is of paramount importance toward the development of therapeutic agents. Accordingly, applicants have endeavored to clone a human MAPKAP-2 kinase, which will prove useful in target-based drug design programs.

Applicants now report the isolation by expression cloning of a novel human serine/threonine signal-transduction kinase described herein. Applicants believe that the newly discovered isolated nucleic acid molecules that encode the novel kinase will allow for the development of therapeutic candidates effective to treat various disorders attending a defective signal-transduction pathway involving a PKAP-2 kinase.

It is noted that the novel kinase of the present invention differs in sequence—at one or more base-contacting amino acid residues—from the known MAPKAP-2 or has a nucleotide base sequence that also differs and is thus distinct from that of a known MAPKAP-2 kinase.

### SUMMARY OF THE INVENTION

Disclosed herein is a newly identified enzyme, MAPKAP-2, which is a serine/threonine signal transduction kinase that is phosphorylated and activated by, inter alia, Erks and p38 MAPK in vitro.

In particular, the present invention relates to nucleic acid molecules coding for MAPKAP-2 kinases; recombinant nucleic acid molecules; cells containing the recombinant nucleic acid molecules; antisense MAPKAP-2 nucleic acid constructs; antibodies raised against such proteins; hybridomas containing the antibodies; nucleic acid probes for the detecting of MAPKAP-2 nucleic acid; a method of detecting MAPKAP-2 nucleic acid or polypeptide in a sample; kits containing nucleic acid probes or antibodies; method(s) of detecting a compound capable of binding to MAPKAP-2 or a fragment thereof; a method of detecting an agonist or antagonist of MAPKAP-2 activity; a method of agonizing or antagonizing MAPKAP-2 associated activity in a mammal; a method of treating pathological conditions associated with over or underexpression of MAPKAP-2 kinase(s) compared to normal in a mammal with an agonist or antagonist of the disclosed polypeptide; and a pharmaceutical composition comprising a MAPKAP-2 agonist or antagonist.

The isolated kinase-encoding nucleic acid molecule preferably comprises the sequences of nucleotides as set forth in SEQ ID NO:1.

Another aspect of the present invention includes a recombinant molecule, comprising a nucleic acid molecule capable of hybridizing under stringent conditions with a nucleic acid sequence as set forth in SEQ ID NO:1, in which the nucleic acid molecule is operatively linked to an expression vector.

Antisense molecule(s) comprising the complement of a polynucleotide comprising the sequence set forth in SEQ ID NO:1 is also provided. In particular, an aspect of the invention is drawn to antisense molecules that have the ability to modulate the transcription/translation of the nucleic acid coding region of SEQ ID NO:1.

Methods to identify compounds that modulate the biological activity of human MAPKAP-2 kinase are also provided.

Allelic variants of the polynucleotide having a sequence of nucleotides as set forth in SEQ ID NO:1 are also provided.

The invention is further directed to an expression vector for the expression of the signal transduction kinase of the invention in a recombinant host cell, wherein the vector contains a nucleic acid molecule comprising a sequence of nucleotides that encode a kinase having an amino acid sequence substantially as depicted in SEQ ID NO:2 or a pharmacologically and/or biologically active or biologically effective derivative thereof.

The invention is further directed to an expression vector for the expression of a biologically effective nucleic acid molecule comprising an antisense nucleic acid sequence derived from SEQ ID NO:1 which has the ability to modulate the transcription/translation of a nucleic acid coding region of the signal transducing polypeptide of the present invention.

Recombinant cells containing the above-described DNAs, mRNA or plasmids i.e., encoding MAPKAP-2 kinase are also provided herein.

Indeed, an aspect of the invention is directed to a host cell containing an expression vector for expression of a signal transducing polypeptide, wherein the vector contains a nucleic acid molecule comprising a sequence of nucleotides which encode the signal transducing polypeptide having the amino acid sequence substantially as set forth in SEQ ID NO:2 or a pharmacologically and/or biologically active or biologically effective derivative thereof.

Yet another aspect of the present invention, is drawn to nucleic acid probes comprising nucleic acid molecules of sufficient length to specifically hybridize to the polynucleotide sequences disclosed herein. The nucleic acid probes of the invention enable one of ordinary skill in the art of genetic engineering to identify and clone similar polypeptides from any species thereby expanding the usefulness of the sequences of the invention.

The present invention is also directed to an isolated and purified nucleic acid molecule derived from SEQ ID NO:1 comprising a sequence of nucleotides that encode a biologically effective dominant negative mutant polypeptide substantially as forth in SEQ ID NO:2 which has the ability to modulate the biological activity and/or pharmacological activity of the MAPKAP-2 kinase of the invention.

The newly discovered member of the protein serine/threonine kinases is capable of regulating signals initiated from a receptor on the surface of a cell, the ability to regulate being dependent upon activation by a member of the MAPK-dependent pathway. The herein disclosed polypeptide comprises at least a portion of an amino acid sequence encoded by a nucleic acid sequence that is capable of hybridizing under stringent conditions with a nucleic acid molecule encoding an amino acid sequence set forth in SEQ ID NO: 2.

Another aspect of the invention contemplates a purified polypeptide comprising the amino acid sequence substantially as set forth in SEQ ID NO:2. Biologically active fragments thereof are also included.

A further aspect of the invention provides assay(s) for screening and the quantitative characterization of potentially pharmacologically effective compounds that specifically interact with and modulate the activity of a signal transducing polypeptide of a signal transduction pathway, particularly the disclosed MAPKAP-2 kinase.

The invention further provides a method of identifying compounds that modulate the biological activity and/or pharmacological activity of a signal transducing molecule, comprising:

(a) combining a candidate compound suspected of modulating signal transduction activity with a polypeptide having an amino acid sequence as set forth in SEQ ID NO:2, and

(b) measuring an effect of the candidate compound modulator on the biological and/or pharmacological activity of the polypeptide.

The invention further provides a method of identifying compounds that modulate the biological and/or pharmacological activity of a signal transducing molecule, comprising:

(a) combining a candidate compound modulator of signal transduction activity with a host-cell expressing a polypeptide having a sequence of amino acids as set forth in SEQ ID NO:2, and

(b) measuring an effect of the candidate compound modulator on the biological and/or pharmacological activity attending the polypeptide. Such a measurement may include detecting changes in level or activity of the polypeptide.

The present invention is also directed to a compound identified by means of one of the aforementioned methods, wherein the compound modulates the biological and/or pharmacological activity of i.e, kinase activity of a MAPKAP-2 kinase.

Further, the invention is directed to a pharmaceutical composition comprising a compound identified by means of one of the aforementioned methods, wherein the compound modulates the biological and/or pharmacological activity of a MAPKAP-2 kinase.

Method(s) of treatment of a disease state manifested by a dysfunctional signal transduction pathway are also provided. One such method comprises administrating an effective amount of a compound identified by means of one of the aforementioned methods, wherein the compound modulates the biological and/or pharmacological activity of a MAPKAP-2 kinase; or administering an effective amount of a biologically effective dominant negative mutant substantially as depicted in SEQ ID NO:2 or a functional derivative thereof.

An alternative embodiment is drawn to a method of treatment of a patient in need of such treatment for a pathophysiological condition mediated by a defective signal-transducing molecule, comprising administration of an effective amount of a biologically effective antisense nucleic acid molecule derived from SEQ ID NO:1; or administering an effective amount of a nucleic acid which encodes a biologically effective dominant negative mutant substantially as depicted in SEQ ID NO:2 or a functional derivative thereof.

In accordance with yet another aspect of the present invention, there are provided antibodies specific for the signal transducing polypeptide comprising the amino acid sequence substantially as depicted in SEQ ID NO:2, as well as a diagnostic composition for the identification of a polypeptide sequence comprising the amino acid sequence substantially as set forth in SEQ ID NO:2.

The invention is also directed to PCR primers derived from SEQ ID NO:1 as well as to methods of making nucleic acid molecules substantially as depicted in SEQ ID NO:1.

Plasmids containing genomic DNA, cDNA or mRNA encoding the MAPKAP-2 kinase polypeptide are also provided.

In accordance with a further aspect of the present invention, there are provided processes for producing the signal transducing kinase of the invention by recombinant techniques comprising culturing transformed prokaryotic and/or eukaryotic host cells, containing nucleic acid sequences encoding the MAPKAP-2 kinase of the invention under conditions promoting expression of the kinase polypeptide(s), followed by subsequent recovery of the polypeptide(s).

In another aspect, the invention provides means for regulating the expression levels of the MAPKAP-2 kinase of the invention thus treating, therapeutically and/or prophylactically, a disorder which can be linked directly or indirectly to the novel kinase disclosed herein.

Also within the invention is a therapeutic composition including, in a pharmaceutically-acceptable carrier, (a) the MAPKAP-2 kinase of the invention, (b) an immunologically active or biologically active fragment thereof, or (c) an antibody having affinity for (a) or (b) above. These therapeutic compositions provide a means for treating various disorders characterized by abnormal (low or ubiquitous) level of expression or activity or a defective signal transducing polypeptide having an amino acid sequence substantially as set forth in SEQ ID NO: 2.

By virtue of having the signal transducing kinase of the invention, agonists or antagonists may be identified which stimulate or inhibit the interaction of a native or recombinant MAPKAP-2 kinase with its binding partner(s), i.e., upstream kinases such as ERK or p38, both of which are known to phosphorylate MAPKAP-2 or downstream target substrates such as Hsp-27 or Hsp-25, which are phosphorylated by activated MAPKAP-2. With either agonists or antagonists the metabolism and reactivity of cells, which express a MAPKAP-2 kinase having an amino acid sequence substantially as set forth of SEQ ID NO: 2, are controlled, thereby providing a means to abate or in some instances prevent the disease of interest.

In accordance with the above, there are provided methods of screening for compounds which bind to and activate (agonist) or inhibit activation (antagonist) of a MAPKAP-2. Indeed, testing of the herein disclosed kinase with a variety of potential agonists or antagonists would provide additional information with respect to the function and activity of the kinase. Such information may lead to the identification of compounds which are capable of very specific interaction with the signal transducing kinase disclosed herein. Such specificity may prove of great value in medical application. Such assays may be designed to identify compounds which bind to the MAPKAP-2 kinase and thereby block or inhibit interaction of the kinase with its binding partner. Other assays can be designed to identify compounds which can stimulate MAPKAP-2-mediated intracellular pathways.

A method of detecting an agonist or antagonist of the herein disclosed human MAPKAP-2 kinase comprises the steps of incubating cells that produce MAPKAP-2 in the presence of a compound and detecting changes in the level of MAPKAP-2 kinase activity.

Another aspect of the invention is drawn to a pharmaceutical composition comprising a MAPKAP-2 agonist or antagonist in an amount sufficient to alter MAPKAP-2 associated kinase activity, and a pharmaceutically acceptable diluent, carrier, or excipient.

In accordance with still another aspect of the present invention, there are provided diagnostic assays for detecting diseases related to mutations in the nucleic acid sequences encoding the novel invention and for detecting an altered level of the encoded polypeptide.

Also, testing of the herein disclosed MAPKAP-2 kinase with a variety of potential agonists or antagonists will provide additional information with respect to the function and activity of the MAPKAP-2 kinase and should lead to the identification and design of compounds that are capable of very specific interaction with native MAPKLAP-2 or its interaction with its binding partner.

The novel nucleic acids disclosed herein, polypeptides encoded by them, vectors, and cells provided herein permit production of a MAPKAP-2 kinase, as well as antibodies to the kinase and antibodies thereto. This provides a means to prepare synthetic or recombinant kinase polypeptides proteins that are substantially free of contamination from many other proteins whose presence can interfere with analysis of a single invention. The availability of the novel MAPKAP-2 makes it possible to observe the effect of a drug substance on the MAPKAP-2 kinase to thereby perform initial in vitro screening of the drug substance in a test system that is specific for the MAPKAP-2 kinase disclosed herein or its native form and its corresponding binding partner.

As well, these also provide diagnostic assays for detecting diseases mediated by a defective signal transduction pathway, particularly one which is attended by a defective signal transducing polypeptide(s) having an amino acid sequence substantially as that set forth in SEQ. ID NO: 2.

The availability of invention—specific antibodies also makes possible the application of the technique of immunohistochemistry to monitor the distribution and expression density of the MAPKAP-2 kinase as well as its corresponding ligand (e.g., in normal vs diseased brain tissue). Such antibodies could also be employed for diagnostic and therapeutic applications. This antibody is preferably capable of neutralizing a biological activity of the polypeptide (i.e. adenylate cyclase activation).

Thus, antibodies, (monoclonal or polyclonal), including purified preparations of an antibody, which is capable of forming an immune complex with the kinase of the invention, such antibody being generated by using as antigen either a polypeptide or a fragment thereof.

As a consequence, an aspect of the invention provides a substantially pure intracellular signal regulated kinase, in which the protein is isolated using an antibody capable of selectively binding to a human kinase polypeptide having the amino acid sequence as set forth in SEQ D NO: 2.

Another aspect of the invention includes an isolated antibody capable of selectively binding to a polypeptide that is phosphorylated by at least one member of a MAPK-dependant pathway, the antibody capable of being produced by a method comprising administering to an animal an effective amount of a substantially pure protein of the present invention, and recovering an antibody capable of selectively binding to the protein.

In accordance with yet a further aspect of the present invention, there are provided processes for utilizing the signal transducing kinase of the invention or nucleic acid molecules encoding such kinase(s) for in vitro purposes such as synthesis of DNA; manufacture of DNA vectors, kinase assays etc.

Further in relation to drug development and therapeutic treatment of various disease states, the availability of polynucleotides encoding the MAPKAP-2 kinase of the invention enables identification of any alterations in such genes (e.g., mutations) which may correlate with the occurrence of certain disease states. In addition, the creation of animal models of such disease states becomes possible, by specifically introducing such mutations into synthetic DNA sequences which can then be introduced into laboratory animals or in vitro assay systems to determine the effects thereof.

At least some of these and other objects are addressed by the various embodiments of the invention disclosed herein. Other features and advantages of the invention will be apparent to those of skill in the art upon further study of the specification and claims.

### DETAILED DESCRIPTION OF THE FIGURES

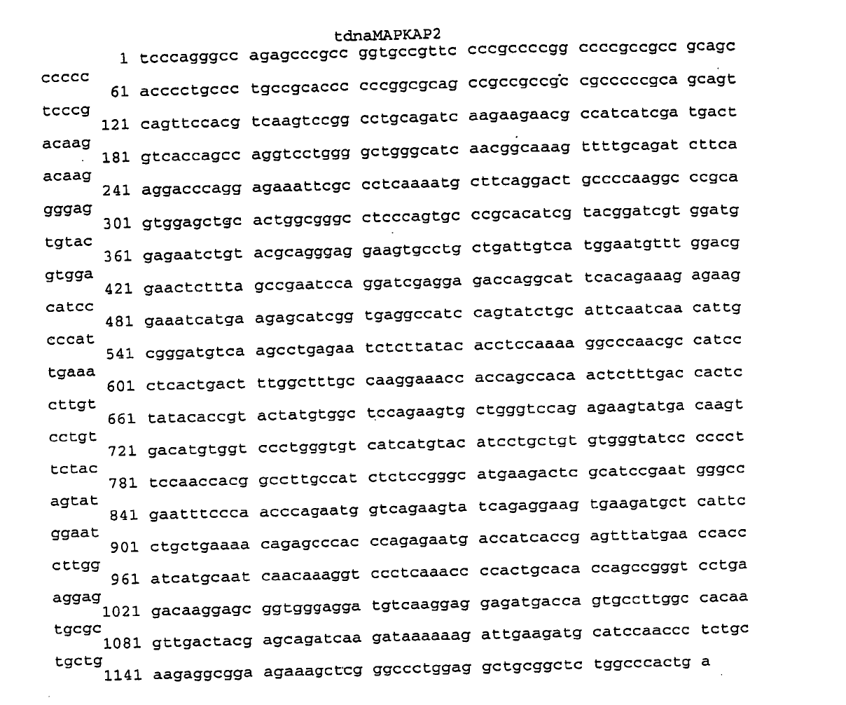

FIG. 1 presents the nucleotide sequence encoding a truncated human MAPKAP-2 kinase of the invention designated tdnaMAPKAP-2 comprising 1191 nucleotides bases in length.

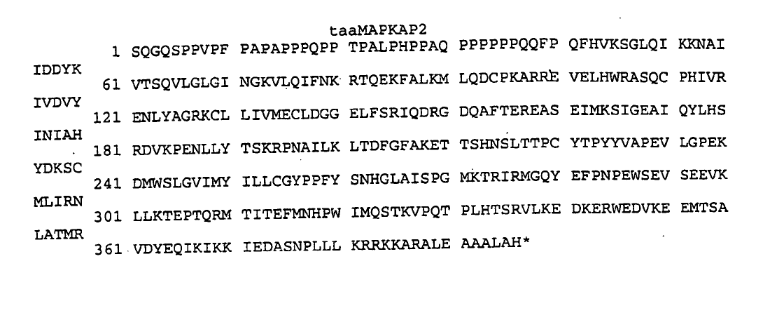

FIG. 2 presents the deduced amino acid sequence of truncated human MAPKAP-2 kinase of the invention, designated taaMAPKAP-2 of 396 amino acids.

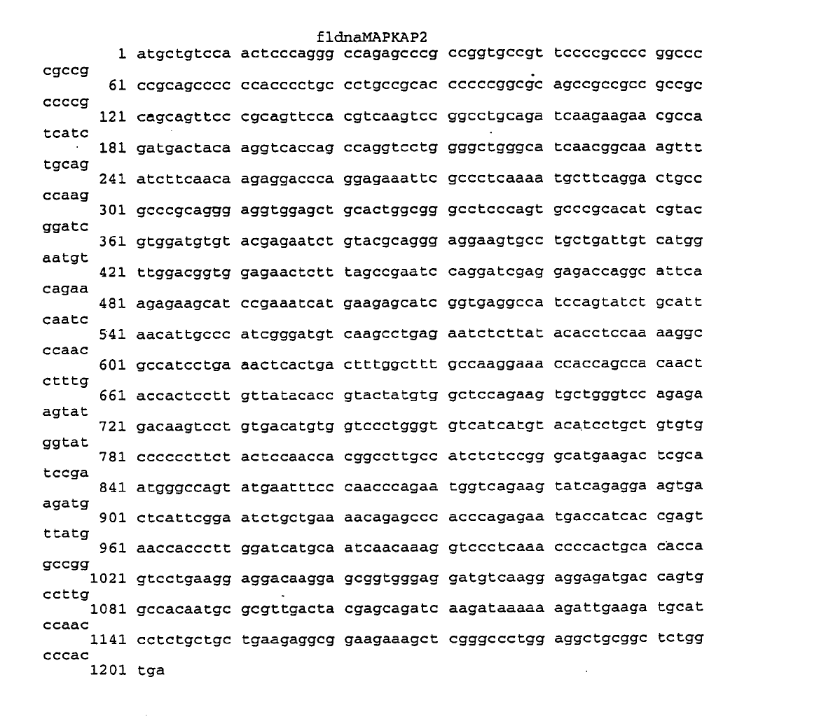

FIG. 3 presents the nucleotide sequence encoding a full length human MAPKAP-2 kinase of the invention designated fldnaMAPKAP-2 comprising 1203 nucleotides bases in length.

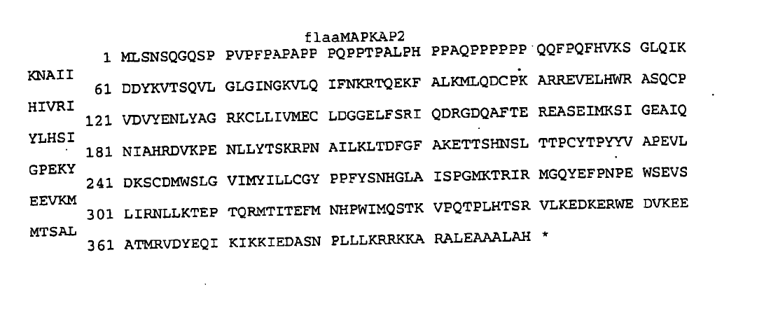

FIG. 4 presents the deduced amino acid sequence of the full length human MAPKAP-2 kinase clone of the invention, designated flaaMAPKAP-2 of 400 amino acids.

### DETAILED DESCRIPTION OF THE INVENTION

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

It is understood that the definition(s) and description(s) following hereunder, while directed to the truncated MAPKAP-2 encoding polynucleotide(s) and the encoded protein(s) (SEQ ID NO:1 and 2 respectively), apply equally to the full length clone (SEQ ID NOs: 3 and 4).

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described.

All publications mentioned herein are incorporated herein by reference for the purpose of describing and disclosing the methodologies, vectors etc which are reported in the publications that might be used in connection with the invention. Nothing herein is to be construed as an admission that the invention is not entitled to antedate such disclosure by virtue of prior invention.

In the description that follows, a number of terms used in the field of recombinant DNA technology are extensively utilized. In order to provide a clearer and consistent understanding of the specification and claims, including the scope to be given such terms, the following definitions are provided.

A “gene” refers to a nucleic acid molecule whose nucleotide sequence codes for a polypeptide molecule. Genes may be uninterrupted sequences of nucleotides or they may include such intervening segments as introns, promoter regions, splicing sites and repetitive sequences. A gene can be either RNA or DNA. A preferred gene is one that encodes the MAPKAP-2 kinase of the present invention.

Use of the terms “isolated” and/or “purified” in the present specification and claims as a modifier of DNA, RNA, polypeptides or proteins means that the DNA, RNA, polypeptides or proteins so designated have been produced in such form by the hand of man, and thus are separated from their native in vivo cellular environment. As a result of this human intervention, the recombinant DNAs, RNAs, polypeptides and proteins of the invention are useful in ways described herein that the DNAs, RNAs, polypeptides or proteins as they naturally occur are not.

Similarly, as used herein, “recombinant” as a modifier of DNA, RNA, polypeptides or proteins means that the DNA, RNA, polypeptides or proteins so designated have been prepared by the efforts of human beings, e.g., by cloning, recombinant expression, and the like. Thus as used herein, recombinant proteins, for example, refers to proteins produced by a recombinant host, expressing DNAs which have been added to that host through the efforts of human beings.

An “insertion” or “addition”, as used herein, refers to a change in an amino acid or nucleotide sequence resulting in the addition of one or more amino acid or nucleotide residues, respectively, as compared to the naturally occurring molecule.

A “substitution”, as used herein, refers to the replacement of one or more amino acids or nucleotides by different amino acids or nucleotides, respectively.

By “signal transduction disorder” is meant any disease or condition associated with an abnormality in a signal transduction pathway. The abnormality may result from a defective MAPKAP-2, kinase or inadequate phosphorylation by its native upstream protein kinase, i.e., mammalian ERK or p38, or low or ubiquitous level of expression of MAPKAP-2 kinase. Alternatively, activated ERK or p38 may not interact effectively with endogenous MAPKAP-2 kinase and thus cause the abnormality in the signal transduction pathway. On the other hand, the level of interaction between them or MAPKAP-2 and any of its native binding partners may be normal, but affecting that interaction may effectively treat a signal transduction pathway disorder.

A “disorder” is any condition that would benefit from treatment with the MAPKAP-2 kinase or nucleic acid encoding such a kinase. This includes chronic and acute disorders or diseases including those pathological conditions which predispose the mammal to the disorder in question. Disorders include, but are not limited to, those of the cardiovascular system, the nervous system and those involving pain perception.

By “abnormality” or “aberrant” is meant a level which is statistically different from the level observed in organisms not suffering from such a disease or condition and may be characterized as either an excess amount, intensity or duration of signal or a deficient amount, intensity or duration of signal. The abnormality in signal transduction may be realized as an abnormality in cell function, viability or differentiation state. An abnormal interaction level may also either be greater or less than the normal level and may impair the normal performance or function of the organism.

By “signal transduction pathway” is meant the sequence of events that involves the transmission of a message from an extracellular protein receptor to the cytoplasm through a cell membrane. The signal ultimately will cause the cell to perform a particular function, for example, to uncontrollably proliferate and therefore cause cancer. Various mechanisms for the signal transduction pathway (Fry et al., Protein Science, 2:1785-1797, 1993) provide possible methods for measuring the amount or intensity of a given signal. Depending upon the particular disease associated with the abnormality in a signal transduction pathway, various symptoms may be detected. Those skilled in the art recognize those symptoms that are associated with the various other diseases described herein. Furthermore, since some adapter molecules recruit secondary signal transducer proteins towards the membrane, one measure of signal transduction is the concentration and localization of various proteins and complexes. In addition, conformational changes that are involved in the transmission of a signal may be observed using circular dichroism and fluorescence studies.

The term “agonist”, as used herein, is meant to refer to an agent that mimics or upregulates (e.g. potentiates or supplements) MAPKAP-2 bioactivity. A MAPKAP-2 agonist can be a wild-type MAPKAP-2 protein or derivative thereof having at least one bioactivity of the wild-type MAPKAP-2. An MAPKAP-2 therapeutic can also be a compound that upregulates expression of an MAPKAP-2 gene or which increases at least one bioactivity of an MAPKAP-2 protein. An agonist can also be a compound, which increases the interaction of an MAPKAP-2 kinase with another molecule, e.g., its substrate. Alternatively, “agonist” refers to a molecule which, when bound to MAPKAP-2 protein, increases or prolongs the duration of the effect of MAPKAP-2.

“Antagonist” as used herein is meant to refer to an agent that downregulates (e.g. suppresses or inhibits) at least one MAPKAP-2 bioactivity. An MAPKAP-2 antagonist can be a compound, which inhibits or decreases the interaction between an MAPKAP-2 kinase and another molecule, e.g., a downstream target molecule. Accordingly, a preferred antagonist is a compound, which inhibits or decreases binding of MAPKAP-2 to either its upstream kinase, e.g., a member of the MAPK that activates MAPKAP-2, or a downstream target substrate that gets activated by MAPKAP-2. An antagonist can also be a compound that down-regulates expression of an MAPKAP-2 gene or which reduces the amount of MAPKAP-2 protein present. The MAPKAP-2 antagonist can be a dominant negative form of an MAPKAP-2 kinase, e.g., a form of an MAPKAP-2 kinase which is capable of interacting with an upstream region of a gene, which is regulated by an MAPKAP-2 transcription factor, but which is not capable of regulating transcription. The MAPKAP-2 antagonist can also be a nucleic acid encoding a dominant negative form of an MAPKAP-2 kinase, an MAPKAP-2 antisense nucleic acid, or a ribozyme capable of interacting specifically with an MAPKAP-2 RNA. Yet other MAPKAP-2 antagonists are molecules which bind to an MAPKAP-2 kinase and inhibit its action. Such molecules include peptides, antibodies and small molecules.

“Dominant negative mutant” as used herein refer to a nucleic acid coding region sequence which has been changed with regard to at least one position in the sequence, relative to the corresponding wild type native version, preferably at a position which changes an amino acid residue position at an active site required for biological and/or pharmacological activity in the native peptide to thereby encode a mutant peptide. Dominant negative mutant as used herein also, in the same manner, may be used to refer to a mutant peptide.

Thus, “dominant negative mutant protein” refers to a mutant protein that interferes with the normal signal transduction pathway. The dominant negative mutant protein contains the domain of interest (e.g., a MAPKAP-2 kinase or a NBP), but has a mutation preventing proper signaling, for example by preventing binding of a second domain from the same protein. One example of a dominant negative protein is described in Millauer et al., Nature Feb. 10, 1994. The agent is preferably a peptide which blocks or promotes interaction of a signal-transducing kinase having a sequence as substantially set forth in SEQ ID NO: 2 and the NBP. The peptide may be recombinant, purified, or placed in a pharmaceutically acceptable carrier or diluent.

The term “modulation” is used herein to refer to the capacity to either enhance or inhibit a biological activity and/or pharmacological activity of a signal transduction molecule having the sequence as substantially set forth in SEQ ID NO: 2 or to the capacity to either enhance or inhibit a functional property of a nucleic acid coding region of the invention nucleic acids. The term “modulate”, as used herein, may also refers to a change or an alteration in the biological activity of MAPKAP-2 kinase.

Modulation may be an increase or a decrease in protein activity, a change in binding characteristics, or any other change in the biological, functional or immunological properties of MAPKAP-2 kinase.

“Biologically effective” as used herein in reference to antisense nucleic acid molecules as well as dominant negative mutant nucleic acid coding regions and dominant negative mutant peptides refers to the ability of these molecules to modulate the biological activity and/or pharmacological activity of the novel signal transduction protein kinase of the present invention and/or transcription/translation of nucleic acid coding regions of the novel signal transduction protein kinase of the present invention.

As used herein, a “functional derivative” of the novel nucleic acid molecule or polypeptide disclosed herein is an entity that possesses a functional biological activity and/or pharmacological activity as defined herein that is derived from SEQ ID NO:1 or SEQ ID NO:2, for example, truncated versions, versions having deletions, functional fragments, versions having substitutions, versions having insertions or extended ends, or biologically effective dominant negative mutants as well as biologically effective antisense molecules.

“Direct administration” as used herein refers to the direct administration of nucleic acid molecules, peptides, or compounds which comprise embodiments and/or functional derivatives (e.g., SEQ ID NO:1 or 2) of the present invention. Direct administration includes but is not limited to gene therapy.

“Antibodies” as used herein includes polyclonal and monoclonal antibodies, chimeric, single chain, and humanized antibodies, as well as Fab fragments, including the products of an Fab or other immunoglobulin expression library.

As used herein, the term “acceptable carrier” encompasses any of the standard pharmaceutical carriers, such as phosphate buffered saline solution, water and emulsions such as an oil/water or water/oil emulsion, and various types of wetting agents.

Invention nucleic acids, oligonucleotides (including antisense), vectors containing same, transformed host cells, polypeptides and combinations thereof, as well as antibodies of the present invention, can be used to screen compounds in vitro to determine whether a compound functions as a potential agonist or antagonist to the MAPKAP-2 kinase of the invention.

Accordingly, methods for identifying compounds, which bind to the MAPKAP-2 kinase polypeptide(s), are also contemplated by the present invention. The kinase(s) of the invention may be employed in a competitive binding assay. Such an assay can accommodate the rapid screening of a large number of compounds to determine which compounds, if any, are capable of binding to the kinase(s) of the invention. Subsequently, more detailed assays can be carried out with those compounds found to bind, to further determine whether such compounds act as modulators, agonists or antagonists of kinase polypeptide(s) of the invention.

As understood by those of skill in the art, assay methods for identifying compounds that modulate kinase activity of the MAPKAP_{—}2 kinase of the invention generally require comparison to a control. One type of a “control” is a cell or culture that is treated substantially the same as the test cell or test culture exposed to the compound, with the distinction that the “control” cell or culture is not exposed to the compound. For example, in methods that use voltage clamp electrophysiological procedures, the same cell can be tested in the presence or absence of compound, by merely changing the external solution bathing the cell. Another type of “control” cell or culture may be a cell or culture that is identical to the transfected cells, with the exception that the “control” cell or culture do not express the invention polypeptide. Accordingly, the response of the transfected cell to compound is compared to the response (or lack thereof) of the “control” cell or culture to the same compound under the same reaction conditions.

In yet another embodiment of the present invention, the activation of MAPKAPS-2 kinase can be modulated by contacting the kinase with an effective amount of at least one compound (agonist or antagonist) identified by the above-described bioassays.

In accordance with another embodiment of the present invention, there are provided methods for diagnosing disease states characterized by abnormal signal transduction. For example, a sample can be obtained from a patient believed to be suffering from a pathological disorder characterized by dysfunctional signal transduction, and contacted with a nucleic acid probe having a sequence of nucleotides that are substantially homologous to the nucleotide sequence set forth in SEQ ID NO:1. Binding of the probe to any complimentary mRNA present in the sample can be determined and is indicative of the regression, progression or onset of such a pathological disorder in the patient.

Alternatively, the patient sample can be contacted with a detectable probe that is specific for the gene product of the invention nucleic acid molecule, under conditions favoring the formation of a probe/gene product complex. The presence of the complex is indicative of the regression, progression or onset of the pathological disorder in the patient.

In accordance with another embodiment of the present invention, there are provided diagnostic systems, preferably in kit form, comprising at least one invention nucleic acid in a suitable packaging material. The diagnostic nucleic acids are derived from the kinase-encoding nucleic acids described herein. In one embodiment, for example, the diagnostic nucleic acids are derived from SEQ D No: 1. Herein disclosed diagnostic systems are useful for assaying for the presence or absence of nucleic acid encoding a MAPKAP-2 kinase either genomic DNA or in transcribed nucleic acid (such as mRNA or cDNA).

A suitable diagnostic system includes at least one invention nucleic acid, preferably two or more invention nucleic acids, as a separately packaged chemical reagent(s) in an amount sufficient for at least one assay. Instructions for use of the packaged reagent are also typically included. Those of skill in the art can readily incorporate invention nucleic probes and/or primers into kit form in combination with appropriate buffers and solutions for the practice of the invention methods as described herein.

As used herein, the term “protein kinase” includes a protein or polypeptide, which is capable of modulating its own phosphorylation state or the phosphorylation state of another protein or polypeptide. Protein kinases can have a specificity for (i.e., a specificity to phosphorylate) serine/threonine residues, tyrosine residues, or both serine/threonine and tyrosine residues, e.g., the dual specificity kinases.

As used herein, the term “mitogen-activating protein kinase” or “MAPK” means a protein kinase which possesses the characteristic activity of phosphorylating and activating other proteins, including but not limited to MAPKAP-2. Examples of MAPK include p38 MAP Kinase, JNK and ERK. These are generally dual specificity kinases.

“Treatment” refers to both therapeutic treatment and prophylactic or preventative measures. Those in need of treatment include those already with the disorder as well as those prone to have the disorder or those in which the disorder is to be prevented.

By “screening” is meant investigating an organism for the presence or absence of a property. The process may include measuring or detecting various properties, including the level of signal transduction and the level of interaction between a MAPKAP-2 kinase, recombinant or naturally occurring and its natural binding partner (NBP)/substrate.

By “NBP” is meant a natural binding partner of a MAPKAP-2 kinase that naturally associates with the polypeptide. The structure (primary, secondary, or tertiary) of the particular natural binding partner will influence the particular type of interaction between the MAPKAP-2 kinase and the natural binding partner. For example, if the natural binding partner comprises a sequence of amino acids complementary to the MAPKAP-2 kinase, covalent bonding may be a possible interaction. Similarly, other structural characteristics may allow for other corresponding interactions. The interaction is not limited to particular residues and specifically may involve phosphotyrosine, phosphoserine, or phosphothreonine residues. A broad range of sequences may be capable of interacting with MAPKAP-2 kinase. Using techniques well known in the art, one may identify several natural binding partners for MAPKAP-2 kinases. NBP also encompasses MAPKAP-2 substrates, natural and synthetic. Yet another aspect of the invention features a method for treatment of an organism having a disease or condition characterized by an abnormality in a signal transduction pathway.

In preferred embodiments the disease or condition which is diagnosed or treated are those described above, the agent is a dominant negative mutant protein provided by gene therapy or other equivalent methods as described below and the agents is therapeutically effective and has an EC.sub.50, or IC.sub.50 as described below.

An EC.sub.50, or IC.sub.50 of less than or equal to 100.mu.M is preferable, and even more preferably less than or equal to 50.mu.M, and most preferably less that or equal to 20.mu.M. Such lower EC.sub.50's or IC.sub.50's are advantageous since they allow lower concentrations of molecules to be used in vivo or in vitro for therapy or diagnosis. The discovery of molecules with such low EC.sub.50's and IC.sub.50's enables the design and synthesis of additional molecules having similar potency and effectiveness. In addition, the molecule may have an EC.sub.50 or IC.sub.50 less than or equal to 100.mu.M at a muscle cell.

By “therapeutically effective amount” is meant an amount of a pharmaceutical composition having a therapeutically relevant effect. A therapeutically relevant effect relieves to some extent one or more symptoms of the disease or condition in the patient; or returns to normal either partially or completely one or more physiological or biochemical parameters associated with or causative of the disease or condition. Generally, a therapeutically effective amount is between about 1 nmole and 1.mu.mole of the molecule, depending on its EC.sub.50 or IC.sub.50, and on the age and size of the patient, and the disease associated with the patient.

Also included in the present invention is a therapeutic compound capable of regulating the activity of a MAPK-dependent pathway in a cell identified by a process, comprising: (a) contacting a cell with a putative regulatory molecule; and (b) determining the ability of the putative regulatory compound to regulate the activity of an MAPK-dependent pathway in the cell by measuring the activation or phosphorylation of at least one substrate specific for at least one member of the MAPK-dependent pathway. The substrate is a human kinase polypeptide having an amino acid sequence substantially the same as that depicted in SEQ. ID. NO: 2. The member is ERK or p38, both of which are members of the MAPK pathway and known to phosphorylate MAPKAP-2.

An alternative embodiment of the invention provides a method for treatment of a disease manifested by a dysfunctional MAPKAP-2 mediated signal transduction pathway which comprises administering to a patient an effective amount of a therapeutic compound comprising at least one regulatory molecule including a molecule capable of decreasing the activity of a MAPK-dependent pathway, a molecule capable of decreasing activity of a polypeptide that is phosphorylated by an activated extracellular signal-regulated kinase, and combinations thereof, in which the effective amount comprises an amount which results in the depletion of harmful cells involved in the disease.

In yet another aspect, the screening assays provided by the invention relate to transgenic mammals whose germ cells and somatic cells contain a nucleotide sequence encoding the MAPKAP-2 kinase disclosed herein or a selected portion of the protein sufficient to activate its native substrate. There are several means by which a sequence encoding, for example, the MAPKAP-2 kinase of the invention may be introduced into a non-human mammalian embryo, some of which are described in, e.g., U.S. Pat. No. 4,736,866, Jaenisch, Science 240-1468-1474 (1988) and Westphal et al., Annu. Rev. Cell Biol. 5:181-196 (1989), which are incorporated herein by reference. The animal's cells then express the receptor and thus may be used as a convenient model for testing or screening selected agonists or antagonists.

I. DNA Constructs Comprising the Polynucleotides Encoding MAPKAP-2; Vectors, Host Cells Containing the DNA constructs, Expression etc.

Provided herein are isolated nucleic acid molecules comprising a sequence of nucleotides that encode a novel human signal transducing kinase—MAPKAP-2 polypeptide. Specifically, isolated cDNA encoding MAPKAP-2 are described as are recombinant messenger RNA (mRNA). Splice variants of the isolated nucleic acid molecules are also described. Typically, unless the MAPKAP-2 of the invention arises as a splice variant, MAPKAP-2-encoding polynucleotides will share substantial sequence homology (i.e., greater than about 90%), with the MAPKAP-2 encoding DNA described herein. DNA or RNA encoding a splice variant may share less than 90% overall sequence homology with the DNA or RNA provided herein, but such a splice variant would include regions of nearly 100% homology to the disclosed DNAs.

The term “nucleic acid” or “nucleic acid molecule” is intended for polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides. DNA can be either complementary DNA (cDNA) or genomic DNA, e.g. a gene encoding the novel kinase disclosed herein.

Unless otherwise indicated, a “nucleotide” defines a monomeric unit of DNA or RNA consisting of a sugar moiety (pentose), a phosphate group, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose, it is referred to as a nucleotide. A sequence of operatively linked nucleotides is typically referred to herein as a “base sequence” or “nucleotide sequence”, and their grammatical equivalents, and is represented herein by a formula whose left to right orientation is in the conventional direction of 5′-terminus to 3′-terminus.

Each “nucleotide sequence” set forth herein is presented as a sequence of deoxyribonucleotides (abbreviated A, G, C and T). However, by “nucleotide sequence” of a nucleic acid molecule is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U). For instance, reference to an RNA molecule having the sequence of SEQ ID NO:1 set forth using deoxyribonucleotide abbreviations is intended to indicate an RNA molecule having a sequence in which each deoxyribonucleotide A, G or C of SEQ ID NO:1 has been replaced by the corresponding ribonucleotide A, G or C, and each deoxyribonucleotide T has been replaced by a ribonucleotide U.

“Invention polynucleotides” or “nucleic acids” refers to (DNA or RNA) containing a nucleotide sequence which encodes the MAPKAP-2 kinase or fragment thereof, or a sequence of nucleotides that hybridize under high stringency conditions to the nucleotide sequences disclosed herein.

As used herein, a “splice variant” refers to variant MAPKAP-2 kinase-encoding nucleic acid(s) produced by differential processing of primary transcript(s) of genomic DNA, resulting in the production of more than one type of mRNA. cDNA derived from differentially processed primary transcript will encode a MAPKAP-2 kinase that has regions of complete amino acid identity and regions having different amino acid sequences. Thus, the same genomic sequence can lead to the production of multiple, related mRNAs and proteins. Both the resulting mRNAs and proteins are referred to herein as “splice variants”.

An “allele” or “allelic sequence”, or “allelic form” as used herein denotes an alternative version of a gene encoding the same functional protein but containing differences in its nucleotide sequence relative to another version of the same gene.

Alleles may result from at least one mutation in the nucleic acid sequence and may result in altered mRNAs or polypeptides whose structure or function may or may not be altered. Any given natural or recombinant gene may have none, one, or many allelic forms. Common mutational changes which give rise to alleles are generally ascribed to natural deletions, additions, or substitutions of nucleotides. Each of these types of changes may occur alone, or in combination with the others, one or more times in a given sequence.

“Allelic polymorphism” or “allelic variant” as used herein denotes a variation in the nucleotide sequence within a gene, wherein different individuals in the general population may express different variants of the gene.

“Consisting essentially of” herein is meant to encompass the disclosed sequence and includes allelic variations of the disclosed nucleotide sequence(s), either naturally occurring or product of in vitro chemical or genetic modification. Each such variant will be understood to have essentially the same nucleotide sequence as the nucleotide sequence of the corresponding MAPKAP-2 disclosed herein.

A “fragment” of a nucleic acid molecule or nucleotide sequence is a portion of the nucleic acid that is less than full-length and comprises at least a minimum length capable of hybridizing specifically with the nucleotide sequence of SEQ ID NO:1 under stringent hybridization conditions. The length of such a fragment is preferably 15-17 nucleotides or more.

A “variant” nucleic acid molecule refers to polynucleotide molecules containing minor changes in the native nucleotide sequence encoding a MAPKAP-2 kinase i.e., changes in which one or more nucleotides of a native sequence is deleted, added, and/or substituted, preferably while substantially maintaining the biological activity of the native nucleic acid molecule. Variant nucleic acid molecules can be produced, for example, by standard DNA mutagenesis techniques or by chemically synthesizing the variant DNA molecule or a portion thereof. Generally, differences are limited so that the nucleotide sequences of the reference and the variant are closely similar overall and, in many regions, identical.

Changes in the nucleotide sequence of a variant polynucleotide may be silent. That is, they may not alter the amino acids encoded by the polynucleotide. Where alterations are limited to silent changes of this type, a variant will encode a polypeptide with the same amino acid sequence as the reference.

Alternatively, the changes may be “conservative.” Conservative variants are changes in the nucleotide sequence that may alter the amino acid sequence of a polypeptide encoded by the reference polynucleotide. Such nucleotide changes may result in amino acid substitutions, additions, deletions, fusions and truncations in the polypeptide encoded by the reference sequence. Thus, conservative variants are those changes in the protein-coding region of the gene that result in conservative change in one or more amino acid residues of the polypeptide encoded by the nucleic acid sequence, i.e. amino acid substitution.

Preferably, a variant form of the preferred nucleic acid molecule has at least 70%, more preferably at least 80%, and most preferably at least 90% nucleotide sequence similarity with the native gene encoding the MAPKAP-2 kinase of the invention.

“Primer” or “nucleic acid polymerize primer(s)” refers to an oligonucleotide, whether natural or synthetic, capable of acting as a point of initiation of DNA synthesis under conditions in which synthesis of a primer extension product complementary to a nucleic acid strand is initiated, i.e., in the presence of four different nucleotide triphosphates and an agent for polymerization (i.e., DNA polymerase or reverse transcriptase) in an appropriate buffer and at a suitable temperature. The exact length of a primer will depend on many factors, but typically ranges from 15 to 25 nucleotides. Short primer molecules generally require cooler temperatures to form sufficiently stable hybrid complexes with the template. A primer need not reflect the exact sequence of the template, but must be sufficiently complementary to hybridize with a template. A primer can be labeled, if desired.

Nucleic acid amplification techniques, which are well known in the art, can be used to locate splice variants of the herein-disclosed MAPKAP-2 kinase. This is accomplished by employing oligonucleotides based on DNA sequences surrounding divergent sequence(s) as primers for amplifying human RNA or genomic DNA. Size and sequence determinations of the amplification products can reveal the existence of splice variants. Furthermore, isolation of human genomic DNA sequences by hybridization can yield DNA containing multiple exons, separated by introns that correspond to different splice variants of transcripts encoding the herein disclosed MAPKAP-2 kinase. Techniques for nucleic-acid manipulation are described generally in, for example, Sambrook et al. (1989) and Ausubel et al. (1987, with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers.

As used herein, a nucleic acid “probe” is single-stranded DNA or RNA, or analog thereof, that has a sequence of nucleotides that includes at least 14, preferably at least 20, more preferably at least 50, contiguous bases that are the same as or the complement of any 14 or more contiguous bases set forth in SEQ ID NO:1. In addition, the entire cDNA-encoding region of the entire sequence corresponding to SEQ ID NO:1 may be used as a probe.

Presently preferred probe-based screening conditions comprise a temperature of about 37° C., a formamide concentration of about 20%, and a salt concentration of about 5× standard saline citrate (SSC; 20×SSC contains 3M sodium chloride, 0.3M sodium citrate, pH 7.0). Such conditions will allow the identification of sequences which have a substantial degree of similarity with the probe sequence, without requiring perfect homology.

“Hybridization” refers to the binding of complementary strands of nucleic acid (i.e., sense:antisense strands or probe:target-DNA) to each other through hydrogen bonds, similar to the bonds that naturally occur in chromosomal DNA. Stringency levels used to hybridize a given probe with target-DNA can be readily varied by those of skill in the art.

The phrase “stringent hybridization conditioned” is used herein to refer to conditions under which polynucleic acid hybrids are stable. As known to those of skill in the art, the stability of hybrids is reflected in the melting temperature (Tₘ) of the hybrids. Tₘ can be approximated by the formula:

81.5° C.−16.6(log₁₀[Na⁺])+0.4l(% G+C)−600/1,

where 1 is the length of the hybrids in nucleotides. Tₘ decreases approximately 1°-1.5° C. with every 1% decrease in sequence homology. In general, the stability of a hybrid is a function of sodium ion concentration and temperature. Typically, the hybridization reaction is performed under conditions of lower stringency, followed by washes of varying, but higher, stringency. Reference to hybridization stringency relates to such washing conditions.

As used herein, the phrase “moderately stringent hybridization” refers to conditions that permit target-DNA to bind a complementary nucleic acid that has about 60% identity, preferably about 75% identity, more preferably-about 85% identity to the target DNA; with greater than about 90% identity to target-DNA being especially preferred. Preferably, moderately stringent conditions are conditions equivalent to hybridization in 50% formamide, 5× Denhart's solution, 5×SSPE, 0.2% SDS at 42° C., followed by washing in 0.2×SSPE, 0.2% SDS, at 65° C.

The phrase “high stringency hybridization” refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in 50% formamide, 5× Denhart's solution, 5×SSPE, 0.2% SDS at 42° C., followed by washing in 0.1×SSPE, and 0.1% SDS at 65° C.

The phrase “low stringency hybridization” refers to conditions equivalent to hybridization in 10% formamide, 5× Denhart's solution, 6×SSPE, 0.2% SDS at 42° C., followed by washing in 1×SSPE, 0.2% SDS, at 50° C.

Denhardt's solution and SSPE (see, e.g., Sambrook, Fritsch, and Maniatis, in: Molecular Cloning, A Laboratory Manual, Cold Spring Harbor Laboratory Press, 1989) are well known to those of skill in the art as are other suitable hybridization buffers. For example, SSPE is pH 7.4 phosphate-buffered 0.18M NaCl. SSPE can be prepared, for example, as a 20× stock solution by dissolving 175.3 g of NaCl, 27.6 g of NaH₂PO₄ and 7.4 g EDTA in 800 ml of water, adjusting the pH to 7.4, and then adding water to 1 liter. Denhardt's solution (see, Denhardt (1966) Biochem. Biophys. Res. Commun. 23:641) can be prepared, for example, as a 50× stock solution by mixing 5 g Ficoll (Type 400, Pharmacia LKB Biotechnology, INC., Piscataway N.J.), 5 g of polyvinylpyrrolidone, and 5 g bovine serum albumin (Fraction V; Sigma, St. Louis Mo.), and then adding water to 500 ml and filtering to remove particulate matter.

Preferred nucleic acids encoding the herein disclosed novel human MAPKAP-2 kinase hybridize under moderately stringent, preferably high stringency, conditions to substantially the entire sequence, or substantial portions (i.e., typically at least 15-30 nucleotides) of the nucleic acid sequence set forth in SEQ ID NO:1.

Preferably, hybridization conditions will be selected which allow the identification of sequences having at least 70% homology with the probe, while discriminating against sequences which have a lower degree of homology with the probe. As a result, nucleic acids having substantially the same nucleotide sequence as the sequence of nucleotides set forth in SEQ ID NO:1 are obtained.

Thus, the nucleic acid probes are useful for various applications. On the one hand, they may be used as PCR primers for amplification of nucleic acid molecules according to the invention. On the other hand, they can be useful tools for the detection of the expression of molecules according to the invention in target tissues, for example, by in-situ hybridization or Northern-Blot hybridization.

The invention probes may be labeled by methods well known in the art, as described hereinafter, and used in various diagnostic kits.

A “label” refers to a compound or composition that facilitates detection of a compound or composition with which it is specifically associated, which can include conferring a property that makes the labeled compound or composition able to bind specifically to another molecule. “Labeled” refers to a compound or composition that is specifically associated, typically by covalent bonding but non-covalent interactions can also be employed to label a compound or composition, with a label. Thus, a label may be detectable directly, i.e., the label can be a radioisotope (e.g., ³H, ¹⁴C, ³²P, ³⁵S, ¹²⁵I, ¹³¹I) or a fluorescent or phosphorescent molecule (e.g., FITC, rhodamine, lanthanide phosphors), or indirectly, i.e., by enzymatic activity (e.g., horseradish peroxidase, beta-galactosidase, luciferase, alkaline phosphatase) or by its ability to bind to another molecule (e.g., streptavidin, biotin, an antigen, epitope, or antibody). Incorporation of a label can be achieved by a variety of means, i.e., by use of radiolabeled or biotinylated nucleotides in polymerase-mediated primer extension reactions, epitope-tagging via recombinant expression or synthetic means, or binding to an antibody.

Labels can be attached directly or via spacer arms of various lengths, i.e., to reduce steric hindrance. Any of a wide variety of labeled reagents can be used for purposes of the present invention. For instance, one can use one or more labeled nucleoside triphosphates, primers, linkers, or probes. A description of immunofluorescent analytic techniques is found in DeLuca, “Immunofluorescence Analysis”, in Antibody As a Tool, Marchalonis et al., eds., John Wiley & Sons, Ltd., pp. 189-231 (1982), which is incorporated herein by reference.

The term label can also refer to a “tag”, which can bind specifically to a labeled molecule. For instance, one can use biotin as a tag and then use avidinylated or streptavidinylated horseradish peroxidase (HRP) to bind to the tag, and then use a chromogenic substrate (e.g., tetramethylbenzamine) to detect the presence of HRP. In a similar fashion, the tag can be an epitope or antigen (e.g., digoxigenin), and an enzymatically, fluorescently, or radioactively labeled antibody can be used to bind to the tag.

In defining nucleic acid sequences, all subject nucleic acid sequences capable of encoding substantially similar amino acid sequences are considered substantially similar or are considered as comprising substantially identical sequences of nucleotides to the reference nucleic acid sequence, i.e., MAPKAP-2 kinase encoding sequence disclosed herein (SEQ ID NO:1).

In practice, the term “substantially the same sequence” means that DNA or RNA encoding two proteins hybridize under moderately stringent conditions and encode proteins that have the same sequence of amino acids or have changes in sequence that do not alter their structure or function.

Nucleotide sequence “similarity” is a measure of the degree to which two polynucleotide sequences have identical nucleotide bases at corresponding positions in their sequence when optimally aligned (with appropriate nucleotide insertions or deletions). Sequence similarity or percent similarity can be determined, for example, by comparing sequence information using sequence analysis software such as the GAP computer program, version 6.0, available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math. 2:482, 1981).

As used herein, “substantially identical sequences of nucleotides” share at least about 90% identity, and substantially identical amino acid sequences share more than 95% amino acid identity. It is recognized, however, that proteins (and DNA or mRNA encoding such proteins) containing less than the above-described level of homology arising as splice variants or that are modified by conservative amino acid substitutions (or substitution of degenerate codons) are contemplated to be within the scope of the present invention.

“Substantially as depicted” or “set forth” as used herein may also refer to functional derivative proteins, and functional derivative nucleic acid sequences as defined herein that may have changes but perform substantially the same biochemical or pharmacological function in substantially the same way; however, “substantially as depicted” as used herein also refers to biologically effective dominant negative mutants and is intended to encompass biologically effective antisense molecules as defined herein.

The present invention also encompasses nucleic acids which differ from the nucleic acid shown in SEQ ID NO:1, but which have the same phenotype. Phenotypically similar nucleic acids are also referred to as “functionally equivalent nucleic acids”.

As used herein, the phrase “functionally equivalent nucleic acids” or “functional derivative” encompasses nucleic acids characterized by slight and non-consequential sequence variations that will function in substantially the same manner to produce the same protein product(s) as the nucleic acids disclosed herein.

Functionally equivalent sequences will function in substantially the same manner to produce substantially the same compositions as the nucleic acid and amino acid compositions disclosed and claimed herein.

In particular, functionally equivalent DNAs encode proteins that are the same as those disclosed herein (SEQ ID NO:2) or that have conservative amino acid variations, such as substitution of a non-polar residue for another non-polar residue or a charged residue for a similarly charged residue. These changes include those recognized by those of skill in the art as those that do not substantially alter the tertiary structure of the protein.

In particular, functionally equivalent nucleic acids encode polypeptides that are the same as those disclosed herein or that have conservative amino acid variations, or that are substantially similar to one having the amino acid sequence as set forth in SEQ ID NO:2.

In one embodiment of the present invention, cDNAs encoding the MAPKAP-2 kinase disclosed herein include substantially the same nucleotide sequence as set forth in SEQ ID NO:1. Preferred cDNA molecules encoding the invention proteins include the same nucleotide sequence as that set forth in SEQ ID NO:1.

Another embodiment of the invention contemplates nucleic acid(s) having substantially the same nucleotide sequence as the reference nucleotide sequence that encodes substantially the same amino acid sequence as that set forth in SEQ ID NO:2.

Further provided are nucleic acids encoding the MAPKAP-2 kinase of the invention those, by virtue of the degeneracy of the genetic code, do not necessarily hybridize to the invention nucleic acids under specified hybridization conditions. Preferred nucleic acids encoding the MAPKAP-2 kinase of the invention are comprised of nucleotides that encode substantially the same amino acid sequence set forth in SEQ ID NO: 2.

As used herein, the term “degenerate” refers to codons that differ in at least one nucleotide from SEQ ID NO:1, but encode the same amino acids as that set forth in SEQ ID NO: 2. For example, codons specified by the triplets “UCU”, “UCC”, “UCA”, and “UCG” are degenerate with respect to each other since all four of these codons encode the amino acid serine.

As used herein, “expression” refers to the process by which polynucleic acids are transcribed into mRNA and translated into peptides, polypeptides, or proteins. If the polynucleic acid is derived from genomic DNA, expression may, if an appropriate eukaryotic host cell or organism is selected, include splicing of the mRNA.

“Expression vector” as used herein refers to nucleic acid vector constructions to direct the transcription of nucleic acid regions in host cells. Expression vectors include but are not limited to plasmids, retroviral vectors, viral and synthetic vectors.

“Transformed host cells” as used herein refer to cells which harbor nucleic acids or functional derivatives of the present invention.

The invention nucleic acids can be produced by a variety of methods well-known in the art, e.g., the methods described herein, employing PCR amplification using oligonucleotide primers from various regions of SEQ ID NO:1 and the like.

Polynucleotides which are identical or sufficiently identical to a nucleotide sequence contained in SEQ ID NO:1, may be used as hybridization probes for cDNA and genomic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than human) that have a high sequence similarity to SEQ ID NO:1. Typically these nucleotide sequences are 70% identical, preferably 80% identical, more preferably 90% identical, most preferably 95% identical to that of the referent. The probes or primers will generally comprise at least 15 nucleotides, preferably, at least 30 nucleotides and may have at least 50 nucleotides. Particularly preferred probes will have between 30 and 50 nucleotides.

Nucleic acid probes derived from the invention polynucleotide sequences are particularly useful for this purpose. Examples of nucleic acids are RNA, cDNA, or isolated genomic DNA encoding the MAPKAP-2 kinase. Such nucleic acids may include, but are not limited to, nucleic acids having substantially the same nucleotide sequence as set forth in SEQ ID NO:1 or one encoding the amino acid sequence as set forth in SEQ ID NO:2. The probes generally will comprise at least 15 nucleotides. Preferably, such probes will have at least 30 nucleotides and may have at least 50 nucleotides. Particularly preferred probes will range between 30 and 50 nucleotides. The probe may be used to isolate splice variants of the polynucleotides disclosed herein.

Thus, one means of isolating a nucleic acid encoding the polypeptide is to probe various sources of human tissue and cDNA such as kidney, liver, skeletal muscle, leukocytes, leukemia MOLT4, and lymphocytes DNA with invention sequences, and then select those sequences having a significant level of sequence homology with the probe employed. Generally, after screening the mammalian library, positive clones are identified by detecting a hybridization signal; the identified clones are characterized by restriction enzyme mapping and/or DNA sequence analysis, and then examined, by comparison with the sequences set forth herein, to ascertain whether they include DNA encoding the entire MAPKAP-2 kinase. If the selected clones are incomplete, they may be used to rescreen the same or a different library to obtain overlapping clones. If desired, the library can be rescreened with positive clones until overlapping clones that encode an entire MAPKAP-2 kinase are obtained. If the library is a cDNA library, then the overlapping clones will include an open reading frame. If the library is genomic, then the overlapping clones may include exons and introns. In both instances, complete clones may be identified by comparison with the DNA and encoded proteins provided herein.

Preferred stringent hybridization conditions include overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 microgram/ml denatured, sheared salmon sperm DNA; followed by washing the filters in 0.1×SSC at about 65° C. Thus the present invention also includes polynucleotides obtainable by screening an appropriate library under stringent hybridization conditions with a labeled probe having the sequence of SEQ ID NO:1 or a fragment thereof.

The skilled artisan will appreciate that, in many cases, an isolated cDNA sequence will be incomplete, in that the region coding for the human polypeptide of the invention is cut short at the 5′ end of the cDNA. This is a consequence of reverse transcriptase, an enzyme with inherently low ‘processivity’ (a measure of the ability of the enzyme to remain attached to the template during the polymerization reaction), failing to complete a DNA copy of the mRNA template during 1st strand cDNA synthesis.

There are several methods available and well known to those skilled in the art to obtain full-length cDNAs, or extend short cDNAs, for example those based on the method of Rapid Amplification of cDNA ends (RACE) (see, for example, Frohman et al., PNAS USA 85, 8998-9002, 1988). Recent modifications of the technique, exemplified by the Marathon.TM. technology (Clontech Laboratories Inc.) for example, have significantly simplified the search for longer cDNAs. In the Marathon.TM. technology, cDNAs have been prepared from mRNA extracted from a chosen tissue and an ‘adaptor’ sequence ligated onto each end. Nucleic acid amplification (PCR) is then carried out to amplify the ‘missing’ 5′ end of the cDNA using a combination of gene specific and adaptor specific oligonucleotide primers. The PCR reaction is then repeated using ‘nested’ primers, that is, primers designed to anneal within the amplified product (typically an adaptor specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Invention DNA sequences or cDNA sequences thus identified can be used for producing MAPKAP-2 kinases, when such nucleic acids are incorporated into a variety of protein expression systems known to those of skill in the art. In addition, such nucleic acid molecules or fragments thereof can be labeled with a readily detectable substituent and used as hybridization probes for assaying for the presence and/or amount of a Invention encoding gene or mRNA transcript in a given sample. The nucleic acid molecules described herein, and fragments thereof, are also useful as primers and/or templates in a PCR reaction for amplifying genes encoding the invention protein described herein.

A polynucleotide encoding the MAPKAP-2 kinase of the present invention, including homologs and orthologs from species other than human, may be obtained by a process which comprises the steps of screening an appropriate library under stringent hybridization conditions with a labeled probe having the sequence of SEQ ID NO:1 or a fragment thereof; and isolating full-length cDNA and genomic clones containing the polynucleotide sequence. Such hybridization techniques are well known to the skilled artisan.

“Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New, York, 1991; and Carillo, H., and Lipman, D., SIAM J Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well-known Smith Waterman algorithm may also be used to determine identity.

Parameters for polynucleotide comparison include the following:

1) Algorithm: Needleman and Wunsch, J. Mol. Biol. 48: 443-453 (1970)

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

A preferred meaning for “identity” for polynucleotides and polypeptides, as the case may be, are provided in (1) and (2) below.

(1) Polynucleotide embodiments further include an isolated polynucleotide comprising a polynucleotide sequence having at least a 50, 60, 70, 80, 85, 90, 95, 97 or 100% identity to the reference sequence of SEQ ID NO:1, wherein the polynucleotide sequence may be identical to the reference sequence of SEQ ID NO:1 or may include up to a certain integer number of nucleotide alterations as compared to the reference sequence, wherein the alterations are selected from the group consisting of at least one nucleotide deletion, substitution, including transition and transversion, or insertion, and wherein the alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein the number of nucleotide alterations is determined by multiplying the total number of nucleotides in SEQ ID NO:1 by the integer defining the percent identity divided by 100 and then subtracting that product from the total number of nucleotides in SEQ ID NO:1, or:

NₙXₙ−(Xₙ Y),

wherein Nₙ is the number of nucleotide alterations, Xₙ is the total number of nucleotides in SEQ ID NO:1, Y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and is the symbol for the multiplication operator, and wherein any non-integer product of Xₙ and Y is rounded down to the nearest integer prior to subtracting it from Xₙ Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

Complementary DNA clones encoding the MAPKAP-2 kinase of the invention may be prepared from the DNA provided. As well, the polynucleotides of the invention can be obtained from natural sources such as genomic DNA libraries or can be synthesized using well-known and commercially available techniques.

Indeed, the invention polynucleotide may be obtained using standard cloning and screening, from a cDNA library derived from mRNA in cells expressing or suspected of expressing native MAPKAP-2 or its native binding partners using the expressed sequence tag (EST) analysis (Adams, M. D., et al. Science (1991) 252:1651-1656; Adams, M. D. et al., Nature, (1992) 355:632-634; Adams, M. D., et al., Nature (1995) 377 Supp:3-174).

The nucleotide sequence encoding the MAPKAP-2 of the invention may be identical over its entire length to the coding sequence set forth in SEQ ID NO: 1, or it may be a degenerate form of this nucleotide sequence encoding the polypeptide of SEQ ID NO:2, or may be highly identical to a nucleotide sequence that encodes the polypeptide of SEQ ID NO:2.

An exemplary nucleic acid molecule encoding the novel human MAPKAP-2 kinase of the invention can be characterized in a number of ways, for example

(i) a nucleic acid molecule may encode the amino acid sequence as set forth in SEQ ID NO:2, or

(ii) a nucleic acid molecule that hybridizes to the DNA of (i) under moderately stringent conditions; or

(iii) a DNA degenerate with respect to either (a) or (b) above, wherein the DNA encodes biologically active MAPKAP-2 kinase; or

(iv) a nucleotide sequence which has at least 75.9% identity to the nucleotide sequence encoding the polypeptide of SEQ ID NO:2; or

(v) the corresponding fragment thereof, or

(vi) a nucleotide sequence which has sufficient identity to the nucleotide sequence contained in SEQ ID NO:1 or allelic variants thereof, splice variants thereof and/or their complements.

Preferably, the nucleic acid molecule(s) of the invention, i.e., SEQ ID NO:1 contains a nucleotide sequence that is highly identical, at least 80% identical, with a nucleotide sequence encoding a human MAPKAP-2 kinase, or at least 85% identical with the encoding nucleotide sequence set forth in SEQ ID NO:1, or at least 90% identical to a nucleotide sequence encoding the polypeptide of SEQ ID NO:2.

Among particularly preferred embodiments of the invention are polynucleotides encoding the MAPKAP-2 kinase of the invention having the amino acid sequence of as substantially set forth in SEQ ID NO:2 and variants thereof.

Further preferred embodiments are polynucleotides encoding MAPKAP-2 polypeptide variants that have the amino acid sequence of the MAPKAP-2 of SEQ ID NO:2 in which several, 5-10, 1-5, 1-3, 1-2 or 1 amino acid residues are substituted, deleted or added, in any combination.

Further preferred embodiments of the invention are polynucleotides that are at least 80% identical over their entire length to a polynucleotide encoding the MAPKAP-2 kinase having the amino acid sequence set out in SEQ ID NO:2, and polynucleotides which are complementary to such polynucleotides. In this regard, polynucleotides at least 80% identical over their entire length to the same are particularly preferred, and those with at least 90% are especially preferred. Furthermore, those with at least 97% are highly preferred and those with at least 98-99% are most highly preferred, with at least 99% being the most preferred.

The present invention further relates to polynucleotides that hybridize to the herein above-described sequences. In this regard, the present invention especially relates to polynucleotides which hybridize under stringent conditions to the herein above-described polynucleotides. As herein used, the term “stringent conditions” means hybridization will occur only if there are at least 95% and preferably at least 97% identity between the sequences.

In another embodiment, the present invention relates to a recombinant DNA molecule comprising, 5′ to 3′, a promoter effective to initiate transcription in a host cell and the above-described invention nucleic acid molecule(s).

The present invention also relates to vectors which comprise a polynucleotide or polynucleotides of the present invention, and host cells which are genetically engineered with vectors of the invention and to the production of polypeptides of the invention by recombinant techniques.

Cell-free translation systems can also be employed to produce such proteins using RNAs derived from the DNA constructs of the present invention.

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (see, e.g., Sambrook et al. (1989) Molecular Cloning: A Laboratory Manual, Second Edition, Cold Spring Harbor Laboratory Press). Suitable means for introducing (transducing) expression vectors containing invention nucleic acid constructs into host cells to produce transduced recombinant cells (i.e., cells containing recombinant heterologous nucleic acid) are well-known in the art (see, for review, Friedmann, 1989, Science, 244:1275-1281; Mulligan, 1993, Science, 260:926-932, each of which are incorporated herein by reference in their entirety).

Exemplary methods of transduction include, e.g., infection employing viral vectors (see, e.g., U.S. Pat. Nos. 4,405,712 and 4,650,764), calcium phosphate transfection (U.S. Pat. Nos. 4,399,216 and 4,634,665), dextran sulfate transfection, electroporation, lipofection (see, e.g., U.S. Pat. Nos. 4,394,448 and 4,619,794), cytofection, particle bead bombardment, and the like. The heterologous nucleic acid can optionally include sequences which allow for its extrachromosomal (i.e., episomal) maintenance, or the heterologous nucleic acid can be donor nucleic acid that integrates into the genome of the host. Recombinant cells can then be cultured under conditions whereby the MAPKAP-2 kinase encoded by the DNA is (are) expressed. Preferred cells include mammalian cells (e.g., HEK 293, CHO and Ltk⁻ cells), yeast cells (e.g., methylotrophic yeast cells, such as Pichia pastoris), bacterial cells (e.g., Escherichia coli), and the like.

Expression vectors for use in carrying out the present invention will comprise a promoter capable of directing the transcription of a cloned DNA and a transcriptional terminator.

Also contained in the expression vectors is a polyadenylation signal located downstream of the coding sequence of interest. Polyadenylation signals include the early or late polyadenylation signals from SV40 (Kaufman and Sharp, ibid.), the polyadenylation signal from the Adenovirus 5 E1B region and the human growth hormone gene terminator (DeNoto et al., Nuc. Acid Res. 9: 3719-3730, 1981). The expression vectors may include a noncoding viral leader sequence, such as the Adenovirus 2 tripartite leader, located between the promoter and the RNA splice sites. Preferred vectors may also include enhancer sequences, such as the SV40 enhancer and the mouse .mu. enhancer (Gillies, Cell 33: 717-728, 1983). Expression vectors may also include sequences encoding the adenovirus VA RNAs.

Suitable expression vectors are well-known in the art, and include vectors capable of expressing DNA operatively linked to a regulatory sequence, such as a promoter region that is capable of regulating expression of such DNA. Thus, an expression vector refers to a recombinant DNA or RNA construct, such as a plasmid, a phage, recombinant virus or other vector that, upon introduction into an appropriate host cell, results in expression of the inserted DNA. Appropriate expression vectors are well known to those of skill in the art and include those that are replicable in eukaryotic cells and/or prokaryotic cells and those that remain episomal or those which integrate into the host cell genome.

Exemplary expression vectors for transformation of E. coli prokaryotic cells include the pET expression vectors (Novagen, Madison, Wis., see U.S. Pat. No. 4,952,496), e.g., pET11a, which contains the T7 promoter, T7 terminator, the inducible E. coli lac operator, and the lac repressor gene; and pET 12a-c, which contains the T7 promoter, T7 terminator, and the E. coli ompT secretion signal. Another such vector is the pIN-IIIompA2 (see Duffaud et al., Meth. in Enzymology, 153:492-507, 1987), which contains the lpp promoter, the lacUV5 promoter operator, the ompA secretion signal, and the lac repressor gene.

Exemplary eukaryotic expression vectors include eukaryotic cassettes, such as the pSV-2 gpt system (Mulligan et al., 1979, Nature, 277:108-114); the Okayama-Berg system (Mol. Cell Biol., 2:161-170), and the expression cloning vector described by Genetics Institute (1985, Science, 228:810-815). Each of these plasmid vectors is capable of promoting expression of the invention chimeric protein of interest.

Also provided are nucleic acid molecule(s) comprising a transcriptional region functional in a cell, a sequence complimentary to an RNA sequence encoding an amino acid sequence corresponding to the herein-disclosed MAPKAP-2 kinase, and a transcriptional termination region functional in a suitable host cell.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Thus, an embodiment provides are transformed host cells that recombinantly express the herein disclosed MAPKAP-2 kinase of the invention.

As used herein, a cell is said to be “altered to express a desired peptide” when the cell, through genetic manipulation, is made to produce a protein which it normally does not produce or which the cell normally produces at lower levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

If desired, the non-coding region 3′ to the sequence encoding an MAPKAP-2 gene may be obtained by the above-described methods. This region may be retained for its transcriptional termination regulatory sequences, such as termination and polyadenylation. Thus, by retaining the 3′-region naturally contiguous to the DNA sequence encoding a MAPKAP-2 kinase, the transcriptional termination signals may be provided. Where the transcriptional termination signals are not satisfactorily functional in the expression host cell, then a 3′ region functional in the host cell may be substituted.

Two DNA sequencers (such as a promoter region sequence and an MAPKAP-2 encoding sequence) are said to be “operably linked” if the nature of the linkage between the two DNA sequences does not (1) result in the introduction of a frame-shift mutation, (2) interfere with the ability of the promoter region sequence to direct the transcription of the MAPKAP-2 encoding gene sequence, or (3) interfere with the ability of the an MAPKAP-2 encoding gene sequence to be transcribed by the promoter region sequence. Thus, a promoter region would be operably linked to a DNA sequence if the promoter were capable of effecting transcription of that DNA sequence.

The selection of control sequences, expression vectors, transformation methods, and the like, are dependent on the type of host cell used to express the gene. As used herein, “cell”, “cell line”, and “cell culture” may be used interchangeably and all such designations include progeny.

The term “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

To express the human MAPKAP-2 kinase-encoding gene of the invention, transcriptional and translational signals recognized by an appropriate host are necessary. The present invention encompasses the expression of the MAPKAPS-2 kinase encoding gene (or a functional derivative thereof) in either prokaryotic or eukaryotic cells.

Host cells which may be used in the expression systems of the present invention are not strictly limited, provided that they are suitable for use in the expression of the novel human MAPKAP-2 kinase of the invention. Suitable hosts may often include eukaryotic cells.

Representative examples of appropriate host cells for use in practicing the present invention include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

Fungal cells, including species of yeast (e.g., Saccharomyces spp., particularly S. cerevisiae, Schizosaccharomyces spp.) or filamentous fungi (e.g., Aspergillus spp., Neurospora spp.) may be used as host cells within the present invention. Suitable yeast vectors for use in the present invention include YRp7 (Struhl et al., Proc. Natl. Acad. Sci. USA. 76: 1035-1039, 1978), YEp13 (Broach et al., Gene 8: 121-133, 1979), POT vectors (Kawasaki et al, U.S. Pat. No. 4,931,373, which is incorporated by reference herein), pJDB249 and pJDB219 (Beggs, Nature 275:104-108, 1978) and derivatives thereof. Such vectors will generally include a selectable marker, which may be one of any number of genes that exhibit a dominant phenotype for which a phenotypic assay exists to enable transformants to be selected. Preferred selectable markers are those that complement host cell auxotrophy, provide antibiotic resistance or enable a cell to utilize specific carbon sources, and include LEU2 (Broach et al., ibid.), URA3 (Botstein et al., Gene 8: 17, 1979), HIS3 (Struhl et al., ibid.) or POT1 (Kawasaki et al., ibid.). Another suitable selectable marker is the CAT gene, which confers chloramphenicol resistance on yeast cells.

Any of a series of yeast gene sequence expression systems can be utilized which incorporate promoter and termination elements from the actively expressed gene sequences coding for glycolytic enzymes are produced in large quantities when yeast are grown in mediums rich in glucose. Known glycolytic gene sequences can also provide very efficient transcriptional control signals.

Yeast provides substantial advantages in that it can also carry out post-translational peptide modifications. A number of recombinant DNA strategies exist which utilize strong promoter sequences and high copy number of plasmids which can be utilized for production of the desired proteins in yeast. Yeast recognizes leader sequences on cloned mammalian gene sequence products and secretes peptides bearing leader sequences (i.e., pre-peptides). For a mammalian host, several possible vector systems are available for the expression of the MAPKAP-2 kinase.

A variety of higher eukaryotic cells may serve as host cells for expression of the polypeptides of the invention, although not all cell lines will be capable of functional coupling of the receptor to the cell's second messenger systems. Cultured mammalian cells, such as BHK, CHO, Y1 (Shapiro et al., TIPS Suppl. 43-46 (1989)), NG108-15 (Dawson et al., Neuroscience Approached Through Cell Culture, Vol. 2, pages 89-114 (1989)), NIE-115 (Liles et al., J. Biol. Chem. 261:5307-5313 (1986)), PC 12 and COS-1 (ATCC CRL 1650) are preferred. Preferred BHK cell lines are the tk.sup.-ts13 BHK cell line (Waechter and Baserga, Proc. Natl. Acad. Sci. USA 79:1106-1110 (1982)) and the BHK 570 cell line (deposited with the American Type Culture Collection, 12301 Parklawn Dr., Rockville, Md. under accession number CRL 10314). A tk.sup.-BHK cell line is available from the ATCC under accession number CRL 1632.

Prokaryotic hosts are, generally, very efficient and convenient for the production of recombinant proteins and are, therefore, one type of preferred expression system for the expressing the MAPKAP-2 kianse encoding gene.

Prokaryotes most frequently are represented by various strains of E. coli. However, other microbial strains may also be used, including other bacterial strains. In prokaryotic systems, plasmid vectors that contain replication sites and control sequences derived from a species compatible with the host may be used. Examples of suitable plasmid vectors may include pBR322, pUC-118, pUC119 and the like; suitable phage or bacteriophage vectors may include .gamma.gt10, .gamma.gt11 and the like; and suitable virus vectors may include pMAM-neo, pKRC and the like. Preferably, the selected vector of the present invention has the capacity to replicate in the selected host cell.

Recognized prokaryotic hosts include bacteria such as E. coli, Bacillus, Streptomyces, Pseudomonas, Salmonella, Serratia, and the like. However, under such conditions, the peptide will not be glycosylated. The prokaryotic host must be compatible with the replicon and control sequences in the expression plasmid.

To express the MAPKAP-2 kinase (or a functional derivative thereof) in a prokaryotic cell, it is necessary to operably link the MAPKAP-2 encoding nucleotide sequence to a functional prokaryotic promoter. Such promoters may be either constitutive or, more preferably, regulatable (i.e., inducible or derepressible). Examples of constitutive promoters and inducible promoters of well known to a skilled artisan. Prokaryotic promoters are reviewed by Cenatiempo (Biochimie 68:505-516 (1986)); and Gottesman (Ann. Rev. Genet. 18:415-442 (1984)). Proper expression in a prokaryotic cell also requires the presence of a ribosome-binding site upstream of the gene sequence-encoding sequence. Such ribosome binding sites are disclosed, for example, by Gold et at., Ann. Rev. Microbiol. 35:365-404 (1981).

As used herein, the term “promoter” refers to a polynucleotide sequence, preferably a DNA sequence, that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

A MAPKAP-2 kinase encoding nucleic acid molecule and an operably linked promoter may be introduced into a recipient prokaryotic or eukaryotic cell either as a nonreplicating DNA (or RNA) molecule, which may either be a linear molecule or, more preferably, a closed covalent circular molecule. Since such molecules are incapable of autonomous replication, the expression of the gene may occur through the transient expression of the introduced sequence. Alternatively, permanent expression may occur through the integration of the introduced DNA sequence into the host chromosome.

In one embodiment, a vector is employed which is capable of integrating the desired gene sequences into the host cell chromosome. Cells which have stably integrated the introduced DNA into their chromosomes can be selected by also introducing one or more markers which allow for selection of host cells which contain the expression vector. The marker may provide for prototrophy to an auxotrophic host, biocide resistance, e.g., antibiotics, or heavy metals, such as copper, or the like. The selectable marker gene sequence can either be directly linked to the DNA gene sequences to be expressed, or introduced into the same cell by co-transfection. Additional elements may also be needed for optimal synthesis of single chain binding protein mRNA. These elements may include splice signals, as well as transcription promoters, enhancers, and termination signals. cDNA expression vectors incorporating such elements include those described by Okayama, Molec. Cell. Biol. 3:280 (1983).

In a preferred embodiment, the introduced nucleic acid molecule will be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Preferred prokaryotic vectors include plasmids such as those capable of replication in E. coli (such as, for example, pBR322, ColE1, pSC101, pACYC 184, .pi.VX. Such plasmids are, for example, disclosed by Sambrook (cf. Molecular Cloning: A Laboratory Manual, second edition, edited by Sambrook, Fritsch, & Maniatis, Cold Spring Harbor Laboratory, (1989)). Bacillus plasmids include pC194, pC221, pT127, and the like. Such plasmids are disclosed by Gryczan (In: The Molecular Biology of the Bacitli, Academic Press, N.Y. (1982), pp. 307-329). Suitable Streptomyces plasmids include p1J101 (Kendall et al., J. Bacteriol. 169:4177-4183 (1987)), and streptomyces bacteriophages such as .phi.C31 (Chater et al., In: Sixth International Symposium on Actinomycetales Biology, Akademiai Kaido, Budapest, Hungary (1986), pp. 45-54). Pseudomonas plasmids are reviewed by John et al. (Rev. Infect. Dis. 8:693-704 (1986)), and Izaki (Jpn. J. Bacteriol. 33:729-742 (1978)).

As noted, supra, expression of the MAPKAP-2 kinase in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J. Mol. Appl. Gen. 1:273-288 (1982)); the TK promoter of Herpes virus (McKnight, Cell 31:355-365 (1982)); the SV40 early promoter (Benoist et al., Nature (London) 290:304-310(1981)); the yeast gal4 gene sequence promoter (Johnston et al., Proc. Natl. Acad. Sci. (USA) 79:6971-6975 (1982); Silver et al., Proc. Natl. Acad. Sci. (USA) 81:5951-5955 (1984)).

As is widely known, translation of eukaryotic mRNA is initiated at the colon which encodes the first methionine. For this reason, it is preferable to ensure that the linkage between a eukaryotic promoter and a DNA sequence which encodes the MAPKAP-2 kinase (or a functional derivative thereof) does not contain any intervening codons which are capable of encoding a methionine (i.e., AUG). The presence of such codons results either in a formation of a fusion protein (if the AUG codon is in the same reading frame as the MAPKAP-2 coding sequence) or a frame-shift mutation (if the AUG codon is not in the same reading frame as the MAPKAP-2 coding sequence).

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274 (1982); Broach, In: The Molecular Biology of the Yeast Saccharomyces: Life Cycle and Inheritance, Cold Spring Harbor Laboratory, Cold Spring Harbor, N.Y., p. 445-470 (1981); Broach, Cell 28:203-204 (1982); Bollon et at., J. Ctin. Hematol. Oncol. 10:3948 (1980); Maniatis, In: Cell Biology: A Comprehensive Treatise, Vol. 3, Gene Sequence Expression, Academic Press, N.Y., pp. 563-608 (1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene molecule(s) results in the production of the herein-disclosed MAPKAP-2 kinase or biologically active fragments thereof. This can take place in the transformed cells as such, or following the induction of these cells to differentiate (for example, by administration of bromodeoxyuracil to neuroblastoma cells or the like).

A variety of incubation conditions can be used to form the peptide of the present invention. The most preferred conditions are those which mimic physiological conditions.

An example of the means for preparing the MAPKAP-2 kinase of the invention is to express nucleic acids encoding the MAPKAP-2 kinase in a suitable host cell, such as a bacterial cell, a yeast cell, an amphibian cell (i.e., oocyte), or a mammalian cell, using methods well known in the art, and recovering the expressed polypeptide, again using well-known methods.

Using methods such as northern blot or slot blot analysis, transfected cells that contain MAPKAP-2 kinase encoding DNA or RNA can be selected. Transfected cells can also be analyzed to identify those that express the MAPKAP-2 kinase. Analysis can be carried out, for example, by using any of well known screening assays attending a functional receptor, and comparing the values obtained to a control, untransfected host cells by electrophysiologically monitoring the currents through the cell membrane in response to MAPKAP-2 kinase, and the like. MAPKAP-2 kinase(s) can be isolated directly from cells that have been transformed with expression vectors comprising nucleic acid encoding the MAPKAP-2 kinases or fragments/portions thereof.

Nucleic acid molecules may be stably incorporated into cells or may be transiently introduced using methods known in the art. Stably transfected mammalian cells may be prepared by transfecting cells with an expression vector comprising a sequence of nucleotides that encodes the MAPKAP-2 kinases in conjunction with a selectable marker gene (such as, for example, the gene for thymidine kinase, dihydrofolate reductase, neomycin resistance, and the like), and growing the transfected cells under conditions selective for cells expressing the marker gene. To prepare transient transfectants, mammalian cells are transfected with a reporter gene (such as the E. coli.beta.-galactosidase gene) to monitor transfection efficiency. The precise amounts and ratios of DNA encoding the MAPKAP-2 kinases may be empirically determined and optimized for a particular cells and assay conditions. Selectable marker genes are typically not included in the transient transfections because the transfectants are typically not grown under selective conditions, and are usually analyzed within a few days after transfection.

In order to identify cells that have integrated the cloned DNA, a selectable marker is generally introduced into the cells along with the gene or cDNA of interest. Preferred selectable markers for use in cultured mammalian cells include genes that confer resistance to drugs, such as neomycin, hygromycin, and methotrexate. The selectable marker may be an amplifiable selectable marker. Preferred amplifiable selectable markers are the DHFR gene and the neomycin resistance gene. Selectable markers are reviewed by Thilly (Mammalian Cell Technology, Butterworth Publishers, Stoneham, Mass., which is incorporated herein by reference). The choice of selectable markers is well within the level of ordinary skill in the art.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA” to the mixture which is introduced into the cells.

In particularly preferred aspects, eukaryotic cells which contain heterologous DNAs express such DNA and form recombinant MAPKAP-2 kinase. In more preferred aspects, recombinant MAPKAP-2 kinase activity is readily detectable because it is a type that is absent from the untransfected host cell.

Heterologous DNA may be maintained in the cell as an episomal element or may be integrated into chromosomal DNA of the cell. The resulting recombinant cells may then be cultured or subcultured (or passaged, in the case of mammalian cells) from such a culture or a subculture thereof. Methods for transfection, injection and culturing recombinant cells are known to the skilled artisan. Similarly, the MAPKAP-2 kinase(s) may be purified using protein purification methods known to those of skill in the art. For example, antibodies or other ligands that specifically bind to the MAPKAP-2 kinase may be used for affinity purification of the MAPKAP-2 kinase.

As used herein, “heterologous or foreign DNA and/or RNA” are used interchangeably and refer to DNA or RNA that does not occur naturally as part of the genome of the cell in which it is present or to DNA or RNA which is found in a location or locations in the genome that differ from that in which it occurs in nature. Typically, heterologous or foreign DNA and RNA refer to DNA or RNA that is not endogenous to the host cell and has been artificially introduced into the cell. Examples of heterologous DNA include DNA disclosed herein.

In other embodiments, mRNA may be produced by in vitro transcription of DNA encoding the MAPKAP-2 kinase. This mRNA can then be injected into Xenopus oocytes where the RNA directs the synthesis of the MAPKAP-2 kinase. Alternatively, the invention-encoding DNA can be directly injected into oocytes for expression of a functional MAPKAP-2 kinase. The transfected mammalian cells or injected oocytes may then be used in the methods of drug screening provided herein.

Alternatively, the invention DNA sequences can be transcribed into RNA, which can then be transfected into amphibian cells for translation into protein. Suitable amphibian cells include Xenopus oocytes.

II. Substantially Pure MAPKAP-2 Polypeptides

Also provided by the present invention are substantially pure signal-transduction kinase polypeptide designated MAPKAP-2. It is of human origin. It can be prepared in any suitable manner. It includes recombinantly produced polypeptides, synthetically produced polypeptides, or polypeptides produced by a combination of these methods.

The MAPKAP-2 kinase of the invention includes the polypeptide defined by the sequence as set forth in SEQ ID NO:2 (in particular the mature polypeptide) as well as those polypeptides which have at least 80% identity to the polypeptide of SEQ ID NO:2 or the relevant portion and more preferably at least 85% identity, and still more preferably at least 90% identity, and even still more preferably at least 95% identity to SEQ ID NO: 2.

The terms “MAPKAP-2 polypeptide” and “MAPKAP-2 protein” “invention polypeptide” “MAPKAP-2 kinase” all of which may be used interchangeably are intended to encompass kinase polypeptides comprising the amino acid sequence shown as SEQ ID NO. 2 or fragments thereof, and homologs thereof and include agonist and antagonist polypeptides.

“Polypeptide” or “peptide” or “protein” refers to a polymer of amino acid residues and to variants and synthetic analogs of the same and are used interchangeably herein. Thus, these terms apply to amino acid polymers in which one or more amino acid residues is a synthetic non-naturally occurring amino acid, such as a chemical analog of a corresponding naturally occurring amino acid, as well as to naturally occurring amino acid polymers. The invention polypeptide is the preferred polypeptide—a MAPKAP-2 kinase having the sequence substantially as set forth in SEQ ID NO:2.

An “amino acid” is a subunit that is polymerized to form proteins and there are twenty amino acids that are universally found in proteins. The general formula for an amino acid is H.sub.2 N—CHR—COOH, in which the R group can be anything from a hydrogen atom (as in the amino acid glycine) to a complex ring (as in the amino acid tryptophan).

The term “protein” refers to a compound formed of 5-50 or more amino acids joined together by peptide bonds.

An “amino acid” is a subunit that is polymerized to form proteins and there are twenty amino acids that are universally found in proteins. The general formula for an amino acid is H.sub.2 N—CHR—COOH, in which the R group can be anything from a hydrogen atom (as in the amino acid glycine) to a complex ring (as in the amino acid tryptophan).

“Reporter” molecules are those radionuclides, enzymes, fluorescent, chemiluminescent, or chromogenic agents which associate with, establish the presence of, and may allow quantification of a particular nucleotide or amino acid sequence.

“Identity,” as known in the art, is a relationship between two or more polypeptide sequences or two or more polynucleotide sequences, as the case may be, as determined by comparing the sequences. In the art, “identity” also means the degree of sequence relatedness between polypeptide or polynucleotide sequences, as the case may be, as determined by the match between strings of such sequences.

“Identity” or “homology” with respect to the invention polypeptide—MAPKAP-2 (SEQ ID NO: 2) is defined herein as the percentage of amino acid residues in the candidate sequence that are identical with the residues in SEQ ID NO: 2, after aligning the sequences and introducing gaps, if necessary, to achieve the maximum percent homology, and not considering any conservative substitutions as part of the sequence identity. No N- nor C-terminal extensions, deletions nor insertions shall be construed as reducing identity or homology.

Parameters for polypeptide sequence comparison include the following:

1) Algorithm: Needleman and Wunsch, J. Mol. Biol. 48: 443-453 (1970) Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Polypeptide embodiments further include an isolated polypeptide comprising a polypeptide having at least a 50, 60, 70, 80, 85, 90, 95, 97 or 100% identity to a polypeptide reference sequence of SEQ ID NO:2, wherein the polypeptide sequence may be identical to the reference sequence of SEQ ID NO: 2 or may include up to a certain integer number of amino acid alterations as compared to the reference sequence, wherein the alterations are selected from the group consisting of at least one amino acid deletion, substitution, including conservative and non-conservative substitution, or insertion, and wherein the alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein the number of amino acid alterations is determined by multiplying the total number of amino acids in SEQ ID NO:2 by the integer defining the percent identity divided by 100 and then subtracting that product from the total number of amino acids in SEQ ID NO:2, or:

Nₐ=Xₐ−(Xₐ Y),

herein Nₐ is the number of amino acid alterations, Xₐ is the total number of amino acids in SEQ ID NO:2, Y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and is the symbol for the multiplication operator, and wherein any non-integer product of Xₐ and Y is rounded down to the nearest integer prior to subtracting it from Xₐ.

As used herein, a “variant” of the invention polypeptide refers to a polypeptide having an amino acid sequence with one or more amino acid substitutions, insertions, and/or deletions compared to the sequence of the invention polypeptide. Generally, differences are limited so that the sequences of the reference (invention polypeptide) and the variant are closely similar overall, and in many regions, identical. Such variants are generally biologically active and necessarily have less than 100% sequence identity with the polypeptide of interest.

In a preferred embodiment, the biologically active variant has an amino acid sequence sharing at least about 70% amino acid sequence identity with the invention polypeptide, preferably at least about 75%, more preferably at least about 80%, still more preferably at least about 85%, even more preferably at least about 90%, and most preferably at least about 95%. Amino acid substitutions are preferably substitutions of single amino-acid residues.

A “fragment” of the invention polypeptide (reference protein) is meant to refer to a protein molecule which contains a portion of the complete amino acid sequence of the wild type or reference protein.

Preferred polypeptides and polynucleotides of the present invention are expected to have, inter alia, similar biological functions/properties to their homologous polypeptides and polynucleotides. Furthermore, preferred polypeptides and polynucleotides of the present invention have at least one MAPKAP-2 related activity.

As used herein, activity of the MAPKAP-2 kinase of the invention (SEQ ID NO: 2) refers to any activity characteristic of invention. Such activity can typically be measured by one or more in vitro methods, and frequently corresponds to an in vivo activity of invention. Such activity may be measured by any method known to those of skill in the art, such as, for example, assays that measure second messenger activity or phosphorylation assays.

The invention polypeptide, biologically active fragments, and functional equivalents thereof can also be produced by chemical synthesis. For example, synthetic polypeptides can be produced using Applied Biosystems, Inc. Model 430A or 431A automatic peptide synthesizer (Foster City, Calif.) employing the chemistry provided by the manufacturer.

The present invention also provides compositions containing an acceptable carrier and any of an isolated, purified invention polypeptide, an active fragment thereof, or a purified, mature protein and active fragments thereof, alone or in combination with each other. These polypeptides or proteins can be recombinantly derived, chemically synthesized or purified from native sources.

The invention polypeptide may be in the form of the “mature” protein or may be a part of a larger protein such as a fusion protein. It is often advantageous to include sequences which aid in purification such as multiple histidine residues, or an additional sequence for stability during recombinant production.

Recombinant MAPKAP-2 kinase of the present invention may be prepared by processes well known in the art from genetically engineered host cells comprising expression systems. Accordingly, in a further aspect, the present invention relates to expression systems which comprise a polynucleotide or polynucleotides of the present invention, host cells which are genetically engineered with such expression systems and to the production of MAPKAP-2 kinase of the invention by recombinant techniques. Cell-free translation systems can also be employed to produce such proteins using RNAs derived from the DNA constructs of the present invention.

Also included are biologically active fragments or variants of the MAPKAP-2 kinase of the invention. A fragment is a polypeptide having an amino acid sequence that entirely is the same as part, but not all, of the amino acid sequence of the aforementioned polypeptide having the amino acid sequence as that depicted in SEQ ID NO:2. Biologically active fragments are those that mediate kinase activity attending the mature or native MAPKAP-2 kinase, including those with a similar activity or an improved activity, or with a decreased undesirable activity. Activity includes not only phosphorylating native substrates, but also synthetic substrates.

The following is a partial list of available substrates that are activated (phosphorylated) by MAPKAP-2 kinase of the invention.

Hsp-27 (Heat-shock protien-27) entire protein sequence, which is well known.

2) Peptides based on Hsp-27

a) LCB-YSRALSRQL-NH2

b) LCB-LLRGPSWDPFR-NH2

c) LCB-RALSRQLSSGV-NH2

Hsp-25

Peptide based on glycogen synthase

a) LCB-KKLNRTLSVA-NH2

4) Peptide based on CREB

a) LCB-KRREILSRRPSYRK

Where LCB=Long chain Biotin and NH2=free amine.

Based upon the sequences provided herein, additional substrates can be identified and designed using known techniques.

As with the invention MAPKAP-2 polypeptides, fragments may be “free-standing,” or comprised within a larger polypeptide of which they form a part or region, most preferably as a single continuous region.

Preferred fragments include, for example, truncation polypeptides having the amino acid sequence substantially the same as that of the disclosed MAPKAP-2 polypeptides, except for deletion of a continuous series of residues that includes the amino terminus, or a continuous series of residues that includes the carboxyl terminus or deletion of two continuous series of residues, one including the amino terminus and one including the carboxyl terminus.

Preferably, all of these polypeptides retain the biological activity of the polypeptide disclosed herein, including its kinase activity. Thus, the polypeptides of the invention include polypeptides having an amino acid sequence at least 80% identical to that of SEQ ID NO:2 or fragments thereof with at least 85% identity to the corresponding fragment of SEQ ID NO:2.

Included in this group are variants of the defined sequence and fragments. Preferred variants are those that vary from the referents by conservative amino acid substitutions—i.e., those that substitute a residue with another of like characteristics. Typical such substitutions are among Ala, Val, Leu and Ile; among Ser and Thr; among the acidic residues Asp and Glu; among Asn and Gin; and among the basic residues Lys and Arg; or aromatic residues Phe and Tyr. Particularly preferred are variants in which several, 5-10, 1-5, or 1-2 amino acids are substituted, deleted, or added in any combination.

III. Peptide Nucleic Acids or “PNAs”

In yet another embodiment, the invention nucleic acid molecules can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of the novel MAPKAP-2 encoding nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of the invention nucleic acid molecules can also be used in the analysis of single base pair mutations in a gene, (e.g., by PNA-directed PCR clamping); as “artificial restriction enzymes” when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of the invention can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of the invention nucleic acid molecules can be generated which may combine the advantageous properties of PNA and DNA.

Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. US. 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

IV. Compositions

The present invention also provides methods for reducing an abnormality in a signal transduction pathway, wherein the signal transduction pathway contains a MAPKAP-2 kinase. Compositions and methods for the treatment of disorders which involve modulating the activity and/or level of individual components, e.g., cell proliferate disorders, hematopoietic cell disorders and disorders of the immune system including inflammation and rheumatoid arthritis, involving the MAPKAP-2 kinase as well as methods for the identification of agents for such treatments are within the scope of the present invention. More, methods and compositions for prognostic evaluation of such disorders are also described.

In terms of methods and compositions for the treatment of such disorders, such methods and compositions may include, but are not limited to the agents capable of decreasing or inhibiting the interaction between a MAPKAP-2 kinase and a MAPKAP-2 target or natural binding partner (NBP). These may include the upstream kinase(s) that phosphorylates MAPKAP-2. Alternatively, the NBP may be a MAPKAP-2 substrate, e.g., Hsp-25 etc. Agents capable of modulating the activity and/or level of interaction between a MAPKAP-2, native or recombinant polypeptide and its binding partner include those agents that inhibit or decrease the dephosphorylating activity of tyrosine phosphatases. Such agents may also include those that increase or stimulate phosphorylating activity of the activated MAPKAP-2 and thus effect its interaction with its native substrate, e.g., Hsp-25 or an artificial substrate.

Methods for the identifying such agents are also described. These methods may include, for example, assays to identify agents capable of disrupting or inhibiting or promoting the interaction between components of the complexes (e.g., MAPKAP-2:NBP complexes), and may also include paradigms and strategies for the rational design of drugs capable of disruption and/or inhibition and/or promotion of such complexes.

A disorder involving a MAPKAP-2: NBP complex may, for example, develop because the presence of such a complex brings about the aberrant inhibition of a normal signal transduction event. In such a case, the disruption of the complex would allow the restoration of the usual signal transduction event. Further, an aberrant complex may bring about an altered subcellular adapter protein localization, which may result in, for example, dysfunctional cellular events. An inhibition of the complex in this case would allow for restoration or maintenance of a normal cellular architecture. Still further, an agent or agents that cause(s) disruption of the complex may bring about the disruption of the interactions among other potential components of a complex.

The above described antibodies may be made specific for recognizing a complex or an epitope thereof, or of specifically recognizing an epitope on either of the components of the complex, especially those epitopes which would not be recognized by the antibody when the component is present separate and apart from the complex. Such antibodies may be used, for example, in the detection of a complex in a biological sample, or, alternatively, as a method for the inhibition of a complex formation, thus inhibiting the development of a disorder. In general, the techniques described above regarding antibodies to MAPKAP-2 may also be used in relation to antibodies to the complex (MAPKAP-2: NBP) and vice versa.

V. Anti-MAPKAP-2 Antibodies and Uses Therefor

Another aspect of the invention pertains to antibodies. For example, by using immunogens derived from a the invention polypeptide(s), its fragments or analogs thereof, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). The subject antibodies, in turn, can be used for producing hybridoma(s), and identifying pharmaceutical compositions, and for studying DNA/protein interaction.

The term “immunospecific” means that the antibodies have substantial greater affinity for the polypeptides of the invention than their affinity for other related polypeptides in the prior art.

The antibodies of the present invention include monoclonal and polyclonal antibodies as well fragments of these antibodies, and humanized forms.

For example, polyclonal and monoclonal antibodies can be produced by methods well known in the art, as described, for example, in Harlow and Lane, Antibodies: A Laboratory Manual (Cold Spring Harbor Laboratory (1988)), which is incorporated herein by reference. Invention polypeptides can be used as immunogens in generating such antibodies. Alternatively, synthetic peptides can be prepared (using commercially available synthesizers) and used as immunogens. Amino acid sequences can be analyzed by methods well known in the art to determine whether they encode hydrophobic or hydrophilic domains of the corresponding polypeptide. Altered antibodies such as chimeric, humanized, CDR-grafted or bifunctional antibodies can also be produced by methods well known in the art. Such antibodies can also be produced by hybridoma, chemical synthesis or recombinant methods described, for example, in Sambrook et al., supra., and Harlow and Lane, supra. Both anti-peptide and anti-fusion protein antibodies can be used. (see, for example, Bahouth et al., Trends Pharmacol. Sci. 12:338 (1991); Ausubel et al., Current Protocols in Molecular Biology (John Wiley and Sons, N.Y. (1989) which are incorporated herein by reference).

Polyclonal antibodies are heterogeneous populations of antibody molecules derived from the sera of animals immunized with an antigen, such as a complex, or an antigenic functional derivative thereof.

A monoclonal antibody, which is a substantially homogeneous population of antibodies to a particular antigen, may be obtained by any technique which provides for the production of antibody molecules by continuous cell lines in culture. These include, but are not limited to the hybridoma technique of Kohler and Milstein (Nature 256:495-497, 1975) and U.S. Pat. No. 4,376,110), the human B-cell hybridoma technique (Kosbor et. al., Immunology Today 4:72, 1983; Cole et al., Proc. Natl. Acad. Sci. USA 80:2026-2030, 1983), and the EBV-hybridoma technique (Cole et al., Monoclonal Antibodies And Cancer Therapy, Alan R. Liss, Inc., 1985, pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

For the production of polyclonal antibodies, various host animals may be immunized by injection with the complex including but not limited to rabbits, mice, rats, etc. Various adjuvants may be used to increase the immunological response, depending on the host species, including but not limited to Freund's (complete and incomplete), mineral gels such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Any animal (mouse, rabbit, and the like) which is known to produce antibodies can be immunized with the selected polypeptide. Methods for immunization are well known in the art. Such methods include subcutaneous or intraperitoneal injection of the polypeptide. One skilled in the art will recognize that the amount of polypeptide used for immunization will vary based on the animal which is immunized, the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or .beta.-galactosidase) or through the inclusion of an adjuvant during immunization. For monoclonal antibodies, spleen cells from the immunized animals are removed, fused with myeloma cells, such as SP2/0-Agl4 myeloma cells, and allowed to become monoclonal antibody producing hybridoma cells.

For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al, MONOCLONAL ANTIBODIES AND CANCER THERAPY, pp. 77-96, Alan R. Liss, Inc., 1985). Active fragments of antibodies are encompassed within the definition of “antibody”.

As the generation of human monoclonal antibodies to the novel MAPKAP-2 antigen may be difficult with conventional techniques, it may be desirable to transfer antigen binding regions of the non-human antibodies, e.g. the F(ab′).sub.2 or hypervariable regions, to human constant regions (Fc) or framework regions by recombinant DNA techniques to produce substantially human molecules. Such methods are generally known in the art and are described in, for example, U.S. Pat. No. 4,816,397, EP publications 173,494 and 239,400, which are incorporated herein by reference. Alternatively, one may isolate DNA sequences which code for a human monoclonal antibody or portions thereof that specifically bind to the human polypeptide by screening a DNA library from human B cells according to the general protocol outlined by Huse et al., Science 246:1275-1281 (1989), incorporated herein by reference, and then cloning and amplifying the sequences which encode the antibody (or binding fragment) of the desired specificity.

Antibody fragments also within the scope of the present invention. These may be generated by known techniques. For example, such fragments include but are not limited to: the F(ab′).sub.2 fragments which can be produced by pepsin digestion of the antibody molecule and the Fab fragments which can be generated by reducing the disulfide bridges of the F(ab′).sub.2 fragments. Alternatively, Fab expression libraries may be constructed (Huse et al., 1989, Science, 246:1275-1281) to allow rapid and easy identification of monoclonal Fab fragments with the desired specificity to the PTK/adapter complex.

Fragments also include single chain antibodies, anti-idiotypic (anti-Id) antibodies, and epitope-binding fragments of any of the above. Techniques for the production of single chain antibodies (U.S. Pat. No. 4,946,778) can also be adapted to produce single chain antibodies to polypeptides of this invention. Alternatively, techniques described for the production of single chain antibodies (U.S. Pat. No. 4,946,778; Bird, Science 242:423-426, 1988; Houston et al., Proc. Natl. Acad. Sci. USA 85:5879-5883, 1988; and Ward et al., Nature 334:544-546, 1989) can be adapted to produce complex-specific single chain antibodies. Single chain antibodies are formed by linking the heavy and light chain fragment of the Fc region via an amino acid bridge, resulting in a single chain polypeptide.

“Humanized antibodies” are also within the scope of the present invention as are chimeric antibodies. Techniques for making “humanized antibodies” are well known and within the skill of one skilled in the art. Humanized forms of the antibodies of the present invention may be generated using one of the procedures known in the art such as chimerization or CDR grafting. As well, transgenic mice, or other organisms including other mammals, may be used to express humanized antibodies. The antibodies are preferably “substantially human” to minimize immunogenicity and are in substantially pure form. By “substantially human” is meant generally containing at least about 70% human antibody sequence, preferably at least about 80% human, and most preferably at least about 90-95% or more of a human antibody sequence to minimize immunogenicity in humans.

Techniques developed for the production of “chimeric antibodies” (Morrison et al., Proc. Natl. Acad. Sci., 81:6851-6855, 1984; Neuberger et al., Nature, 312:604-608, 1984; Takeda et al., Nature, 314:452-454, 1985) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

In another embodiment, the present invention relates to a method of detecting an MAPKAP-2 kinase in a sample, comprising: a) contacting the sample with an above-described antibody, under conditions such that immunocomplexes form, and b) detecting the presence of said antibody bound to the polypeptide. In detail, the methods comprise incubating a test sample with one or more of the antibodies of the present invention and assaying whether the antibody binds to the test sample. Altered levels of MAPKAP-2 in a sample as compared to normal levels may indicate muscular disease. Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion based Ouchterlony, or rocket immunofluorescent assays) can readily be adapted to employ the antibodies of the present invention. Examples of such assays can be found in Chard, “An Introduction to Radioimmunoassay and Related Techniques” Elsevier Science Publishers, Amsterdam, The Netherlands (1986); Bullock et al., “Techniques in Immunocytochemistry,” Academic Press, Orlando, Fla. Vol. 1 (1982), Vol. 2 (1983), Vol. 3 (1985); Tijssen, “Practice and Theory of Enzyme Immunoassays: Laboratory Techniques in Biochemistry and Molecular Biology,” Elsevier Science Publishers, Amsterdam, The Netherlands (1985).

The immunological assay test samples of the present invention include cells, protein or membrane extracts of cells, or biological fluids such as blood, serum, plasma, or urine. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is capable with the system utilized.

In another embodiment, the present invention relates to a hybridoma which produces the above-described monoclonal antibody, or binding fragment thereof. A hybridoma is an immortalized cell line which is capable of secreting a specific monoclonal antibody. In general, techniques for preparing monoclonal antibodies and hybridomas are well known in the art (Campbell, “Monoclonal Antibody Technology: Laboratory Techniques in Biochemistry and Molecular Biology,” Elsevier Science Publishers, Amsterdam, The Netherlands (1984); St. Groth et al., J. Immunol. Methods 35:1-21 (1980)).

Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell Res. 175:109-124 (1988)). Hybridomas secreting the desired antibodies are cloned and the class and subclass is determined using procedures known in the art (Campbell, Monoclonal Antibody Technology: Laboratory Techniques in Biochemistry and Molecular Biology, supra (1984)). For polyclonal antibodies, antibody-containing antisera is isolated from the immunized animal and is screened for the presence of antibodies with the desired specificity using one of the above-described procedures.

An alternative embodiment contemplates detectably labeling the above-described antibodies. For example, a detectable marker can be directly or indirectly attached to the antibody. Useful markers include, for example, radioisotopes, affinity labels (such as biotin, avidin, and the like), enzymatic labels (such as horseradish peroxidase, alkaline phosphatase, and the like) fluorescent labels (such as FITC or rhodamine, and the like), paramagnetic atoms, and the like. Procedures for accomplishing such labeling are well known in the art, for example, see (Stemberger et al., J. Histochem. Cytochem. 18:315 (1970); Bayer et at., Meth. Enzym. 62:308 (1979); Engval et al., Immunot. 109:129 (1972); Goding, J. Immunol. Meth. 13:215 (1976)). The labeled antibodies of the present invention can be used for in vitro, in vivo, and in situ assays to identify cells or tissues which express a specific peptide.

Accordingly, methods are contemplated herein for detecting the presence of the novel polypeptides on the surface of a cell. In one assay format invention polypeptide is identified and/or quantified by using labeled antibodies, preferably monoclonal antibodies which are reacted with body tissue known to express high levels hMAPKAP-2 and determining the specific binding thereto, the assay typically being performed under conditions conducive to immune complex formation. Unlabeled primary antibody can be used in combination with labels that are reactive with primary antibody to detect the receptor. For example, the primary antibody may be detected indirectly by a labeled secondary antibody made to specifically detect the primary antibody. Alternatively, the anti-MAPKAP-2 antibody can be directly labeled, as described above. A wide variety of labels may be employed, such as radionuclides, particles (e.g., gold, ferritin, magnetic particles, red blood cells), fluorophores, chemiluminescers, enzymes, enzyme substrates, enzyme cofactors, enzyme inhibitors, ligands (particularly haptens), etc.

In another embodiment of the present invention the above-described antibodies are immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir et al., “Handbook of Experimental Immunology” 4th Ed., Blackwell Scientific Publications, Oxford, England, Chapter 10 (1986); Jacoby et al., Meth. Enzym. 34 Academic Press, N.Y. (1974)). The immobilized antibodies of the present invention can be used for in vitro, in vivo, and in situ assays as well as in immunochromotograph.

“Immunologically active fragment(s)” of the invention polypeptides are also embraced by the invention. Such fragments are those proteins that are capable of raising MAPKAP-2-specific antibodies in a target immune system (e.g., murine or rabbit) or of competing with native MAPKAP-2 for binding to invention-polypeptide specific antibodies, and is thus useful in immunoassays for detecting the presence of MAPKAP-2 kinases in a biological sample. Such immunologically active fragments typically have a minimum size of 8 to 11 consecutive amino acids.

Furthermore, one skilled in the art can readily adapt currently available procedures, as well as the techniques, methods and kits disclosed above with regard to antibodies, to generate peptides capable of binding to a specific peptide sequence in order to generate rationally designed antipeptide peptides, for example see Hurby et al., “Application of Synthetic Peptides: Antisense Peptides”, In Synthetic Peptides, A User's Guide, W. H. Freeman, N.Y., pp. 289-307(1992), and Kaspczak et al., Biochemistry 28:9230-8 (1989).

Anti-peptide peptides can be generated in one of many ways. A an aside, the anti-peptide peptides can be generated by replacing the basic amino acid residues found in the MAPKAP-2 kinase sequence with acidic residues, while maintaining hydrophobic and uncharged polar groups. For example, lysine, arginine, and/or histidine residues are replaced with aspartic acid or glutamic acid and glutamic acid residues are replaced by lysine, arginine or histidine.

Such antibodies can also be used for the immunoaffinity or affinity chromatography purification of the invention polypeptides. Antibodies so produced can also be used, inter alia, in diagnostic methods and systems to detect the level of the invention polypeptide(s) present in a mammalian, preferably human, body sample, such as tissue. With respect to the detection of such polypeptides, the antibodies can be used for in vitro diagnostic or in vivo imaging methods.

Immunological procedures useful for in vitro detection of invention polypeptides in a sample include immunoassays that employ a detectable antibody. Such immunoassays include, for example, competitive assays, sandwich assays, and the like, as generally described in, e.g., U.S. Pat. Nos. 4,642,285; 4,376,110; 4,016,043; 3,879,262; 3,852,157; 3,850,752; 3,839,153; 3,791,932; and Harlow and Lane, Antibodies, A Laboratory Manual, Cold Spring Harbor Publications, N.Y. (1988), each incorporated by reference herein. Also included are Pandex microfluorimetric assay, agglutination assays, flow cytometry, serum diagnostic assays and immunohistochemical staining procedures, which are well known in the art.

Thus, an anti-MAPKAP-2 antibody (e.g., monoclonal antibody) can be used to isolate native MAPKAP-2 by standard techniques, such as affinity chromatography or immunoprecipitation. An anti-MAPKAP-2 antibody can facilitate the purification of native MAPKAP-2 from cells and of recombinantly produced MAPKAP-2 expressed in host cells. Moreover, an anti-MAPKAP-2 antibody can be used to detect MAPKAP-2 protein (e.g., in a cellular lysate or cell supernatant) in order to evaluate the abundance and pattern of expression of the MAPKAP-2 protein. Anti-MAPKAP-2 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include .sup.125 I, .sup.131 I, .sup.35 S or .sup.3H.

There are a variety of assay formats known to those of ordinary skill in the art for using an antibody to detect a polypeptide in a sample. See, e.g., Harlow and Lane, Antibodies: A Laboratory Manual, Cold Spring Harbor Laboratory, 1988. For example, the antibody may be immobilized on a solid support such that it can bind to and remove the polypeptide from the sample. The bound polypeptide may then be detected using a second antibody that binds to the antibody/peptide complex and contains a detectable reporter group. Alternatively, a competitive assay may be utilized, in which polypeptide that binds to the immobilized antibody is labeled with a reporter group and allowed to bind to the immobilized antibody after incubation of the antibody with the sample. The extent to which components of the sample inhibit the binding of the labeled polypeptide to the antibody is indicative of the level of polypeptide within the sample. Suitable reporter groups for use in these methods include, but are not limited to, enzymes (e.g., horseradish peroxidase), substrates, cofactors, inhibitors, dyes, radionuclides, luminescent groups, fluorescent groups and biotin.

In another embodiment of the present invention, a kit is provided which contains all the necessary reagents to carry out the previously described methods of detection. The kit may comprise: i) a first container means containing an above-described antibody, and ii) second container means containing a conjugate comprising a binding partner of the antibody and a label.

In an alternative embodiment, the kit further comprises one or more other containers comprising one or more of the following: wash reagents and reagents capable of detecting the presence of bound antibodies. Examples of detection reagents include, but are not limited to, labeled secondary antibodies, or in the alternative, if the primary antibody is labeled, the chromophoric, enzymatic, or antibody binding reagents which are capable of reacting with the labeled antibody. The compartmentalized kit may be as described above for nucleic acid probe kits.

One skilled in the art will readily recognize that the antibodies described in the present invention can readily be incorporated into one of the established kit formats which are well known in the art.

VI. Uses and Methods of the Invention

The invention polypeptide(s) (kinase polypeptide) are hypothesized to be ubiquitous in the mammalian host and are responsible for many biological functions, including many pathologies. Accordingly, it is desirous to find compounds and drugs which stimulate invention polypeptide on the one hand and which can inhibit the function of invention polypeptide on the other hand.

Specifically, the nucleic acid molecules, proteins, protein homologs, and antibodies described herein can be used in one or more of the following methods: a) screening assays; b) predictive medicine i.e., diagnostic assays, prognostic assays, monitoring clinical trials and c) method of treatment, i.e., therapeutic and prophylactic.)

The invention nucleic acids can be used, for example, to express the invention polypeptides, to detect MAPKAP-2 mRNA or a genetic alteration in MAPKAP-2 encoding gene, and to modulate MAPKAP-2 activity, as described below. The MAPKAP-2 kinases can be used to treat disorders characterized by insufficient or excessive expression of MAPKAP-2 kinase or its native substrate or production of MAPKAP-2 kinase specific inhibitors or agonists. As well, the invention polypeptide may be used to screen for naturally occurring MAPKAP-2 substrates, screen for therapeutics or compounds that modulate MAPKAP-2 activity, or production of MAPKAP-2 kinases that have decreased or aberrant activity compared to wild type MAPKAP-2. More, the anti-MAPKAP-2 antibodies may be useful for detecting and isolating MAPKAP-2 kinases, imaging, regulating bioavalability of MAPKAP-2 polypeptides, and modulating MAPKAP-2 activity. It is understood that the term “MAPKAP-2 polypeptide or kinase” is preferably the polypeptide having the sequence substantially as set forth in SEQ ID NO:2.

A. Screening Assays

The signal-transduction kinase described herein—invention polypeptide or MAPKAP-2 kinase, its immunogenic fragments or oligopeptides can be used for screening therapeutic compounds in any of a variety of drug screening techniques. The fragment employed in such a test may be free in solution, affixed to a solid support, borne on a cell surface, or located intracellularly.

Cell-Based Assays

Cell based assays can be used for identifying modulators, i.e., candidate or test compounds or agents (e.g., peptides, peptidomimetics, small molecules or other drugs) which bind to MAPKAP-2 kinases, have a stimulatory or inhibitory effect on, for example, MAPKAP-2 expression or MAPKAP-2 activity, or have a stimulatory or inhibitory effect on, for example, the expression or activity of a MAPKAP-2 substrate.

As used herein, a compound or a signal that “modulates the activity” of invention polypeptide refers to a compound or a signal that alters the activity of invention polypeptide so that the activity of the invention polypeptide is different in the presence of the compound or signal than in the absence of the compound or signal. In particular, such compounds or signals include agonists and antagonists. Such activity is generally detected using conventional assays described herein.

Examples of methods for the synthesis of molecular libraries can be found in the art, for example in: DeWitt et al. Proc. Natl. Acad. Sci. U.S.A. 0.90:6909 (1993); Zuckermann et al. J. Med. Chem. 37:2678 (1994).

Libraries of compounds may be presented in solution (e.g., Houghten Biotechniques 13:412-421 (1992), or on beads (Lam, Nature 354:82-84 (191)), chips (Fodor Nature 364:555-556 (1993)), bacteria (U.S. Pat. No. 5,223,409), spores (U.S. Pat. No. '409), plasmids (Cull et al. Proc Natl Acad Sci USA 89:1865-1869 (1992)) or on phage (Scott and Smith, Science 249:386-390 (1990)).

Thus, methods for screening for compounds which modulate the expression of DNA or RNA encoding recombinant or native MAPKAP-2 kinase, as well as the function of the invention polypeptide in vivo are included within the scope of the present invention. Compounds which modulate these activities may be DNA, RNA, peptides, proteins, or non-proteinaceous organic molecules. Compounds may modulate by increasing or attenuating the expression of DNA or RNA encoding the novel signal-transduction kinase polypeptide, or the function thereof. Compounds that modulate the expression of DNA or RNA encoding the invention polypeptide or the function of the polypeptide may be detected by a variety of assays.

The assay may be a simple “yes/no” assay to determine whether there is a change in expression or function. The assay may be made quantitative by comparing the expression or function of a test sample with the levels of expression or function in a standard sample.

Consequently, an embodiment of the invention promises methods of identifying compounds that modulate the activity of a signal-transduction kinase polypeptide, which comprise combining a candidate compound suspected of being a modulator of a signal-transduction kinase activity with a signal-transduction kinase having the sequence substantially as depicted in SEQ ID NO:2, and measuring an effect of the candidate compound modulator on the kinase activity, i.e. ability of MAPKAP-2 to phosphorylate a MAPKAP-2 substrate such as Hsp-27.

Accordingly, in one aspect, the invention features methods for identifying a reagent which modulates MAPKAP-2 kinase activity, comprising incubating MAPKAP-2 kinase with the test reagent and measuring the effect of the test reagent on the ability of MAPKAP-2 kinase to phosphorylate a MAPKAP-2 substrate.

Alternatively, methods of identifying compounds that modulate the activity of a signal-transduction kinase, comprise combining a candidate compound modulator of a signal-transduction kinase activity with a host-cell expressing the signal-transduction kinase molecule having the sequence substantially as depicted in SEQ ID NO:2, and measuring an effect of the candidate compound modulator on the kinase activity. Preferred cellular assays for inhibitors of the kinase fall into two general categories: 1) direct measurement of the kinase activity, and 2) measurement of downstream events in the signaling cascade. These methods can employ the endogenous kinase, or the overexpressed recombinant kinase. Thus, activation of the MAPKAP-2 signal-transduction pathway may include measuring MAPKAP-2 kinase activity by the rate of substrate phosphorylation, e.g., Hsp-27 as determined by quantification of the rate of ³²P incorporation. Hsp-27 or another suitable MAPKAP-2 substrate may be co-expressed by the host cell, or in the alternative it may be added exogenously.

The term “MAPKAP-2 substrate” as used herein include MAPKAP-2 substrates, e.g., native substrates such as Hsp-27, Hsp-25 as well as artificial substrates well known to a skilled artisan.

The term “modulation of MAPKAP-2 activity” includes inhibitory or stimulatory effects. The invention is particularly useful for screening reagents that inhibit MAPKAP-2 activity. Such reagents are useful for the treatment or prevention of MAPKAP-2-mediated disorders, for example, inflammation and oxidative damage.

MAPKAP-2 kinase assays, for use in evaluating the polypeptide variants and other agents discussed herein, include any assays that evaluate a compound's ability to phosphorylate Hsp-27 or other MAPKAP-2 substrates, thereby rendering the MAPKAP-2 active (i.e., capable of phosphorylating in vivo substrates such as Hsp-27). MAPKAP-2 kinase for use in such methods may be endogenous proteins or variants thereof, may be purified or recombinant, and may be prepared using any of a variety of techniques that will be apparent to those of ordinary skill in the art.

For example, cDNA-encoding MAPKAP-2 may be cloned by PCR amplification from a suitable human cDNA library, using polymerase chain reaction (PCR) and methods well known to those of ordinary skill in the art. MAPKAP-2 may be cloned using primers based on the published sequence (Derijard et al., Science 267:682-685, 1995). MAPKAP-2 cDNA may then be cloned into a bacterial expression vector and the protein produced in bacteria, such as E. coli, using standard techniques. The bacterial expression vector may, but need not, include DNA encoding an epitope such as glutathione-S transferase protein (GST) such that the recombinant protein contains the epitope at the N- or C-terminus.

The ability of the MAPKAP-2 kinase to phosphorylate a MAPKAP-2 target molecule/substrate can be determined by, for example, an in vitro kinase assay. Briefly, a MAPKAP-2 substrate molecule, e.g., an immunoprecipitated MAPKAP-2 substrate molecule (Hsp-27, for example) from a cell line expressing such a molecule, can be incubated with the MAPKAP-2 kinase and radioactive ATP, e.g., [.δ³²P] ATP, in a suitable buffer containing 50 mM HEPES pH 8, 10 mM MgCl.sub.2, 1 mM DTT, 100.mμ.M ATP) for 60 minutes at 30° C. In general, approximately 50 ng to 1.mu.g of the polypeptide and 50 ng recombinant MAPKAP-2, with 2-7 cpm/fmol [.χ³²P]ATP, is sufficient. Following the incubation, the immunoprecipitated MAPKAP-2 substrate molecule can be separated by SDS-polyacrylamide gel electrophoresis under reducing conditions, transferred to a membrane, e.g., a PVDF membrane, and autoradiographed. The appearance of detectable bands on the autoradiograph indicates that the MAPKAP-2 substrate i.e., Hsp-27 has been phosphorylated.

Incorporation of [³²P]phosphate into the MAPKAP-2 target substrate may be quantitated using techniques well known to those of ordinary skill in the art, such as with a phosphorimager. To evaluate the substrate specificity of polypeptide variants, a kinase assay may generally be performed as described above except that other MAPKAP-2 substrates are substituted for the Hsp-27.

Phosphoaminoacid analysis of the phosphorylated substrate can also be performed in order to determine which residues on the MAPKAP-2 substrate are phosphorylated. Briefly, the radiophosphorylated protein band can be excised from the SDS gel and subjected to partial acid hydrolysis. The products can then be separated by one-dimensional electrophoresis and analyzed on, for example, a phosphoimager and compared to ninhydrin-stained phosphoaminoacid standards.

In order to measure the cellular activity of the kinase, the source may be a whole cell lysate, prepared by one to three freeze-thaw cycles in the presence of standard protease inhibitors. Alternatively, the kinase may be partially or completely purified by standard protein purification methods. Finally, the kinase may be purified by affinity chromatography using specific antibody for the C terminal regulatory domain described herein or by ligands specific for the epitope tag engineered into the recombinant kinase moreover described herein. The kinase preparation may then be assayed for activity as described in the prior art.

To determine whether MAPKAP-2 phosphorylation results in activation, a coupled in vitro kinase assay may be performed using a substrate for MAPKAP-2, such as Hsp-27, with or without an epitope tag. It should be noted that alternative buffers may be used and that buffer composition can vary without significantly altering kinase activity. Reactions may be separated by SDS-PAGE, visualized by autoradiography and quantitated using any of a variety of known techniques. Activated MAPKAP-2 will be capable of phosphorylating Hsp-27 at a level that is at least 5% above background using such an assay.

The specificity of MAPKAP-2 substrate phosphorylation can be tested not only by measuring Hsp-27 activation, but instead by employing mutated Hsp-27 molecules that lack the sites of MAPKAP-2 phosphorylations. Altered phosphorylation of the substrate relative to control values indicates alteration of the MAPKAP-2 signal transduction pathway, and increased risk in a subject of an MAPKAP-2-mediated disorder.

An embodiment of the invention pertains to detecting an active MAPKAP-2 kinase in a sample, in which an immunokinase assay is employed. Briefly, polyclonal or monoclonal antibodies may be raised against a unique sequence of a MAPKAP-2 kinase using standard techniques. A sample to be tested, such as a cellular extract, is incubated with the anti-MAPKAP-2 antibodies to immunoprecipitate a MAPKAP-2 kinase, and the immunoprecipitated material is then incubated with a substrate (e.g., Hsp-27) under suitable conditions for substrate phosphorylation. The level of substrate phosphorylation may generally be determined using any of a variety of assays, as described herein.

Methods of identifying compounds that modulate the biological activity of a signal-transduction kinase are also preferred, which comprise combining a candidate compound modulator of a signal-transduction kinase activity with a signal-transduction kinase having the sequence substantially as depicted in SEQ ID NO:2, and measuring an effect of the candidate compound modulator on the biological activity, which may include its synthesis, function, or activity.

In one embodiment, the screening assay comprises contacting a cell transfected with a reporter gene operably linked to an MAPKAP-2 promoter with a test compound and determining the level of expression of the reporter gene. The reporter gene can encode, e.g., a gene product that gives rise to a detectable signal such as: color, fluorescence, luminescence, cell viability, relief of a cell nutritional requirement, cell growth, and drug resistance. For example, the reporter gene can encode a gene product selected from the group consisting of chloramphenicol acetyl transferase, luciferase, beta-galactosidase and alkaline phosphatase.

Methods of identifying compounds that modulate the pharmacological activity of a signal-transduction kinase are also preferred, which comprise combining a candidate compound modulator of a signal-transduction kinase activity with a signal-transduction kinase having the sequence substantially as depicted in SEQ ID NO:2, and measuring an effect of the candidate compound modulator on the pharmacological activity.

In addition to detecting MAPKAP-2 kinase activity in a sample, methods for detecting the level of MAPKAP-2 in a sample are also provided. The level of a MAPKAP-2 kinase or polynucleotide may generally be determined using a reagent that binds to the MAPKAP-2 kinase, DNA or mRNA.

An exemplary cell-based assay is based upon determining the ability of the test compound to modulate activity of MAPKAP-2 wherein the step of determining the ability of the test compound to modulate activity of MAPKAP-2 is accomplished, for example, by determining the ability of a signal-transduction kinase molecule having the sequence substantially as depicted in SEQ ID NO:2, to bind to or interact with the test compound or reagent. To detect MAPKAP-2 kinase, the reagent is typically an antibody, which may be prepared as described herein.

In a preferred embodiment, determining the ability of the MAPKAP-2 kinase to bind to or interact with a MAPKAP-2 target molecule can be accomplished by determining the activity of the target molecule. For example, the activity of the target molecule (substrate) can be determined by detecting induction of a cellular second messenger of the target (e.g., intracellular Ca.sup.2+, diacylglycerol, IP.sub.3, etc.), detecting catalytic/enzymatic activity of the target and appropriate substrate, detecting the induction of a reporter gene (comprising a target-responsive regulatory element operatively linked to a nucleic acid encoding a detectable marker, e.g., chloramphenicol acetyl transferase), or detecting a target-regulated cellular response.

Compounds which are identified generally according to methods described, contemplated, and referenced herein that modulate the biological and/or pharmacological activity of a signal-transduction molecule of the sequence substantially as depicted in SEQ ID NO:2 are especially preferred embodiments of the present invention.

Another embodiment provides a method for screening a plurality of compounds for specific binding affinity with the signal-transduction kinase polypeptide or a fragment thereof, comprising providing

i) a plurality of compounds;

ii) combining the invention polypeptide or a fragment thereof with each of a plurality of compounds for a time sufficient to allow binding under suitable conditions; and

iii) detecting binding of the kinase polypeptide, or fragment thereof, to each of the plurality of compounds, thereby identifying the compounds which specifically bind the signal-transduction kinase polypeptide.

A still further embodiment of the present invention provides a method of detecting an agonist or antagonist of MAPKAP-2 activity comprising incubating cells that express a polypeptide having the sequence substantially as set forth in SEQ ID NO: 2 in the presence of a compound and detecting changes in the level of polypeptide activity. The compounds thus identified would produce a change in activity indicative of the presence of the compound. In a preferred embodiment, the compound is present within a complex mixture, for example, serum, body fluid, or cell extracts. Once the compound is identified it can be isolated using techniques well known in the art.

In a preferred embodiment, the method includes the steps of (a) forming a reaction mixture, which includes: (i) a polypeptide having the sequence substantially as set forth on SEQ ID NO:2, (ii) a MAPKAP-2 binding partner/substrate and (iii) a test compound; and (b) detecting interaction between the polypeptide and the binding partner. A statistically significant change (potentiation or inhibition) in the interaction of the polypeptide and binding partner in the presence of the test compound, relative to the interaction in the absence of the test compound, indicates a potential agonist (mimetic or potentiator) or antagonist (inhibitor) of MAPKAP-2 bioactivity for the test compound. The reaction mixture can be a cell-free protein preparation, e.g., a reconstituted protein mixture or a cell lysate, or it can be a recombinant cell including a heterologous nucleic acid recombinantly expressing the MAPKAP-2 binding partner.

In preferred embodiments, the step of detecting interaction of the MAPKAP-2 and MAPKAP-2 binding partner is a competitive binding assay. In other preferred embodiments, at least one of the MAPKAP-2 polypeptide and the MAPKAP-2 binding partner comprises a detectable label, and interaction of the MAPKAP-2 and MAPKAPP-2 binding partner is quantified by detecting the label in the complex. The detectable label can be, e.g., a radioisotope, a fluorescent compound, an enzyme, or an enzyme co-factor. In other embodiments, the complex is detected by an immunoassay.

In a further embodiment, the present invention provides a method of agonizing (stimulating) or antagonizing MAPKAP-2 associated activity in a mammal comprising administering to said mammal an agonist or antagonist to a polypeptide having the sequence substantially as set forth in SEQ ID NO:2 in an amount sufficient to effect said agonism or antagonism.

A preferred embodiment of the present invention is a method for treatment of a patient in need of such treatment for a condition which is mediated by the human signal-transduction polypeptide described herein, e.g., SEQ ID NO:2, comprising administration of a therapeutically effective amount of a human signal-transduction modulating compound identified using sequence substantially as depicted in SEQ ID NO:2 as a pharmacological target in methods contemplated herein.

In a preferred embodiment, the present invention relates to a method of treating inflammatory related diseases including rheumatoid arthritis in a mammal with an agonist or antagonist of MAPKAP-2 activity comprising administering the agonist or antagonist to a mammal in an amount sufficient to agonize or antagonize MAPKAP-2 associated functions.

Cell-Free Assays

Cell-free assays can also be used to identify compounds capable of interacting with a MAPKAP-2 kinase or its binding partner or substrate, to thereby modify activity of the polypeptide or binding partner. Such a compound can effect the activity of the polypeptide. Cell-free assays will also find use in identifying compounds which modulate the interaction between a MAPKAP-2 kinase and its natural binding partner/target molecule/substrate, i.e., Hsp-27.

An exemplary cell-free screening assay includes the steps of contacting a MAPKAP-2 kinase polypeptide or functional fragment thereof with a test compound or library of test compounds under conditions favoring formation of a complex there between and detecting the formation of complexes. For detection purposes, the polypeptide can be labeled with a specific marker and the test compound or library of test compounds labeled with a different marker. Interaction of a test compound with the MAPKAP-2 kinase or fragment thereof partner can then be detected by determining the level of the two labels after an incubation step and a washing step. The presence of two labels after the washing step is indicative of an interaction.

In another embodiment, the assay includes the steps of contacting the MAPKAP-2 kinase polypeptide or biologically active portion thereof with a known compound which binds the MAPKAP-2 kinase to form an assay mixture, contacting the assay mixture with a test compound, and determining the ability of the test compound to interact with the MAPKAP-2 kinase, wherein determining the ability of the test compound to interact with the polypeptide comprises determining the ability of the test compound to preferentially bind to the MAPKAP-2 kinase polypeptide or biologically active portion thereof as compared to the known compound.

In an alternative cell-free assay, a MAPKAP-2 kinase polypeptide or biologically active portion thereof is contacted with a test compound and the ability of the test compound to modulate (e.g., stimulate or inhibit) the activity of the MAPKAP-2 kinase or biologically active portion thereof is determined. Determining the ability of the test compound to modulate the activity of a MAPKAP-2 kinase can be accomplished, for example, by determining the ability of the MAPKAP-2 kinase to bind to a MAPKAP-2 target molecule (substrate) by one of the methods described above for determining direct binding.

Determining the ability of the MAPKAP-2 kinase polypeptide to bind to a MAPKAP-2 target molecule can also be accomplished using a technology such as real-time Biomolecular Interaction Analysis (BIA). Sjolander, S. and Urbaniczky, C. Anal. Chem. 63:2338-2345 (1991). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

An alternative embodiment to determine the ability of a compound to modulate the interaction between MAPKAP-2 kinase and its target molecule, without the labeling of any of the interactants, proposes the use of a microphysiometer to detect the interaction of MAPKAP-2 kinase with its target molecule. A microphysiometer is useful in that it allows detection of the interaction without the labeling of either MAPKAP-2 or the target molecule. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between the compound and its binding partner.

In more than one embodiment of the above assay methods of the present invention, it may be desirable to immobilize either MAPKAP-2 kinase or its target molecule to facilitate separation of complexed from uncomplexed forms of the proteins, as well as to accommodate automation of the assay. Binding of a test compound to the MAPKAP-2 kinase, or interaction of a MAPKAP-2 kinase with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/MAPKAP-2 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or MAPKAP-2 kinase, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of MAPKAP-2 kinase binding activity determined using standard techniques.

Other techniques for immobilizing proteins on matrices can also be used in the screening assays of the invention. For example, either a MAPKAP-2 kinase or a MAPKAP-2 target molecule can be immobilized utilizing conjugation of biotin and streptavidin. Biotinylated MAPKAP-2 kinase or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical).

Alternatively, antibodies reactive with MAPKAP-2 kinase or target molecules but which do not interfere with binding of the MAPKAP-2 kinase to its target molecule can be derivatized to the wells of the plate, and unbound target or MAPKAP-2 kinase trapped in the wells by antibody conjugation. Methods for detecting such complexes, in addition to those described above for the GST-immobilized complexes, include immunodetection of complexes using antibodies reactive with the MAPKAP-2 kinase or target molecule, as well as enzyme-linked assays which rely on detecting an enzymatic activity associated with the MAPKAP-2 kinase or target molecule.

One such method included within the scope of the invention is a method for identifying an agent to be tested for an ability to modulate a signal transduction pathway disorder. The method involves exposing at least one agent to a MAPKAP-2 kinase for a time sufficient to allow binding of the agent to the protein; removing non-bound agents; and determining the presence of the compound bound to the protein, thereby identifying an agent to be tested for an ability to modulate a disorder involving a MAPKAP-2 kinase complex.

In an alternative embodiment, determining the ability of the test compound to modulate the activity of a MAPKAP-2 kinase can be accomplished by determining the ability of the MAPKAP-2 kinase to further modulate the activity of a MAPKAP-2 target molecule (e.g., a MAPKAP-2 mediated signal transduction pathway component—Hsp-27 for example). For example, the activity of the effector molecule on an appropriate target can be determined, or the binding of the effector to an appropriate target can be determined as previously described.

In yet another embodiment, modulators of MAPKAP-2 expression are identified in a method wherein a cell is contacted with a candidate compound and the expression of MAPKAP-2 mRNA or protein in the cell is determined. The level of expression of MAPKAP-2 mRNA or protein in the presence of the candidate compound is compared to the level of expression of MAPKAP-2 mRNA or protein in the absence of the candidate compound. The candidate compound can then be identified as a modulator of MAPKAP-2 expression based on this comparison. For example, when expression of MAPKAP-2 mRNA or polypeptide is greater (statistically significantly greater) in the presence of the candidate compound than in its absence, the candidate compound is identified as a stimulator (agonist) of MAPKAP-2 mRNA or polypeptide expression. Alternatively, when expression of MAPKAP-2 mRNA or polypeptide is less (statistically significantly less) in the presence of the candidate compound than in its absence, the candidate compound is identified as an inhibitor (antagonist) of MAPKAP-2 mRNA or polypeptide expression. The level of MAPKAP-2 mRNA or polypeptide expression in the cells can be determined by methods described herein for detecting MAPKAP-2 mRNA or polypeptide.

In yet another aspect of the invention, the MAPKAP-2 kinase can be used as “bait proteins” in a two-hybrid assay or three-hybrid assay (see, e.g., U.S. Pat. No. 5,283,317; Zervos et al. Cell 72:223-232 (1993); and Brent WO94/10300), to identify other proteins, which bind to or interact with MAPKAP-2 (“MAPKAP-2-binding proteins” or “MAPKAP-2-bp”) and are involved in MAPKAP-2 activity. Such MAPKAP-2-binding proteins are also likely to be involved in the propagation of signals by the MAPKAP-2 kinases or MAPKAP-2 targets as, for example, downstream elements of a MAPKAP-2-mediated signaling pathway. Alternatively, such MAPKAP-2-binding proteins are likely to be MAPKAP-2 inhibitors.

The two-hybrid system is based on the modular nature of most transcription factors, which consist of separable DNA-binding and activation domains. Briefly, the assay utilizes two different DNA constructs. In one construct, the gene that codes for a MAPKAP-2 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a MAPKAP-2-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene, which encodes the protein, which interacts with the MAPKAP-2 kinase.

Accordingly, it is within the scope of this invention to further use an agent identified as described herein in an appropriate animal model. For example, an agent identified as described herein (e.g., a MAPKAP-2 modulating agent, an antisense MAPKAP-2 nucleic acid molecule, a MAPKAP-2-specific antibody, or a MAPKAP-2-binding partner) can be used in an animal model to determine the efficacy, toxicity, or side effects of treatment with such an agent.

Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) detect MAPKAP-2 encoding nucleotide sequences in a sample; (ii) map their respective genes on a chromosome, and, thus, locate gene regions associated with genetic disease; and (iii) identify an individual from a minute biological sample (tissue typing). These applications are described in the subsections below. Gene products can likewise be used as herein described.

(i) Detection in a Sample

To detect nucleic acid encoding a polypeptide having the sequence substantially as set forth in SEQ ID NO: 2, standard hybridization and/or PCR techniques may be employed using a nucleic acid probe or a PCR primer. Suitable probes and primers may be designed by those of ordinary skill in the art based on the MAPKAP-2 cDNA sequences provided herein.

Consequently, determining the level of MAPKAP-2 expression may be accomplished by Northern blot analysis. Polyadenylated [poly(A)+] mRNA is isolated from a test sample. The mRNA is fractionated by electrophoresis and transferred to a membrane. The membrane is probed with labeled MAPKAP-2 cDNA. In another embodiment, MAPKAP-2 expression is measured by quantitative PCR applied to expressed mRNA.

In another embodiment, the test reagent is incubated with a cell transfected with an MAPKAP-2 polynucleotide expression vector, and the effect of the test reagent on MAPKAP-2 transcription is measured by Northern blot analysis, as described above.

In another embodiment, activation of the MAPKAP-2 signal transduction pathway is determined by measuring the level of MAKPAK-2 expression in a test sample. In a specific embodiment, the level of MAPKAP-2 expression is measured by Western blot analysis. The proteins present in a sample are fractionated by gel electrophoresis, transferred to a membrane, and probed with labeled antibodies to MAPKAP-2.

Determining the ability of the MAPKAP-2 kinase to bind to or interact with a MAPKAP-2 target molecule can be accomplished by determining direct binding. Determining the ability of the MAPKAP-2 kinase to bind to or interact with a MAPKAP-2 target molecule can be accomplished, for example, by coupling the MAPKAP-2 kinase with a radioisotope or enzymatic label such that binding of the MAPKAP-2 kinase to a MAPKAP-2 target molecule can be determined by detecting the labeled MAPKAP-2 kinase in a complex. For example, MAPKAP-2 molecules, e.g., MAPKAP-2 kinase, can be labeled with ¹²⁵I, ³⁵S, ¹⁴C, or ³H, either directly or indirectly, and the radioisotope detected by direct counting of radioemmission or by scintillation counting.

Alternatively, MAPKAP-2 kinase molecules can be enzymatically labeled with, for example, horseradish peroxidase, alkaline phosphatase, or luciferase, and the enzymatic label detected by determination of conversion of an appropriate substrate to product.

(ii) Chromosome Mapping

The nucleic acid molecules of the present invention may be valuable for chromosome identification. The sequence(s) is specifically targeted to and can hybridize with a particular location on an individual human chromosome. The mapping of relevant sequences to chromosomes according to the present invention is an important first step in correlating those sequences with gene associated disease. Upon mapping a sequence to a precise chromosomal location, the physical position of the sequence on the chromosome can be correlated with genetic map data. (Such data are found, for example, in V McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. Nature, 325:783-787 (1987). The differences in the cDNA or genomic sequence between affected and unaffected individuals can also be determined. If a mutation is observed in some or all of the affected individuals but not in any normal individuals, then the mutation is likely to be the causative agent of the disease.

For example, upon isolation of a sequence (or a portion of the sequence) of a gene, the isolated sequence can be used to map the location of the gene on a chromosome. This process is called chromosome mapping. Accordingly, portions or fragments of the MAPKAP-2 encoding nucleotide sequence(s) (SEQ ID NO:1), described herein, can be used to map the location of the MAPKAP-2 encoding genes on a chromosome.

As an example, MAPKAP-2 encoding genes can be mapped to chromosomes by preparing PCR primers (preferably 15-25 bp in length) from the herein disclosed MAPKAP-2 nucleotide sequences. Computer analysis of the MAPKAP-2 sequences can be used to predict primers that do not span more than one exon in the genomic DNA, thus complicating the amplification process. Thereafter, these primers can be used for PCR screening of somatic cell hybrids containing individual human chromosomes. Only those hybrids containing the human gene corresponding to the MAPKAP-2 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel will contain either a single human chromosome or a small number of human chromosomes, and a full set of mouse chromosomes, allowing easy mapping of individual genes to specific human chromosomes. (D'Eustachio P. et al. (1983) Science 220:919-924).

Somatic cell hybrids containing only fragments of human chromosomes can also be produced by using human chromosomes with translocations and deletions. PCR mapping of somatic cell hybrids is a rapid procedure for assigning a particular sequence to a particular chromosome. Three or more sequences can be assigned per day using a single thermal cycler. Using the MAPKAP-2 nucleotide sequences to design oligonucleotide primers, sublocalization can be achieved with panels of fragments from specific chromosomes. Other mapping strategies include in situ hybridization (described in Fan, Y. et al. Proc. Natl. Acad. Sci. USA, 87:6223-27(1990)), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle.

The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

More, differences in the DNA sequences between individuals affected and unaffected with a disease associated with the MAPKAP-2 encoding gene, can be determined. If a mutation is observed in some or all of the affected individuals but not in any unaffected individuals, then it is save to assume that the mutation is likely the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

(iii) Tissue Typing

The MAPKAP-2 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the MAPKAP-2 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The MAPKAP-2 nucleotide sequences of the invention uniquely represent portions of the human genome.

Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals.

If a panel of reagents from MAPKAP-2 nucleotide sequences described herein is used to generate a unique identification database for an individual, those same reagents can later be used to identify tissue from that individual. Using the unique identification database, positive identification of the individual, living or dead, can be made from extremely small tissue samples.

The MAKAP-2 nucleotide sequences described herein can further be used to provide polynucleotide reagents, e.g., labeled or labelable probes which can be used in, for example, an in situ hybridization technique, to identify a specific tissue, e.g., brain tissue.

In a similar fashion, the above referenced probes or MAPKAP-2 primers can also be used to screen tissue culture for contamination (i.e. screen for the presence of a mixture of different types of cells in a culture).

C. Predictive Medicine

The invention further features predictive medicines, which features diagnostic assays, prognostic assays and monitoring clinical trials used for prognostic (predictive) purposes with a view towards treating an individual prophylactically are based, at least in part, on the identity of the novel MAPKAP-2 gene and alterations in the genes and related pathway genes, which affect the expression level and/or function of the encoded MAPKAP-2 kinase in a subject.

Detection of a mutated form of a human MAPKAP-2 encoding gene associated with a dysfunction will provide a diagnostic tool that can add to or define a diagnosis of a disease or susceptibility to a disease which results from under-expression, over-expression or altered expression of human MAPKAP-2 kinase. Individuals carrying mutations in the MAPKAP-2 encoding gene may be detected at the DNA level by a variety of techniques.

Information obtained using the diagnostic assays described herein (alone or in conjunction with information on another genetic defect, which contributes to the same disease) is useful for prognosing, diagnosing or confirming that a subject has a genetic defect (e.g. in a MAPKAP-2 gene or in a gene that regulates the expression of a MAPKAP-2 gene), which causes or contributes to the development of inflammatory related disorders. Based on prognostic information, a doctor can recommend a regimen (e.g. diet or exercise) or therapeutic protocol, which is useful for preventing or prolonging onset of a pathological condition characterized by aberrant expression of a MAPKAP-2 gene in the individual.

In addition, knowledge of the particular alteration or alterations, resulting in defective or deficient MAPKAP-2 gene(s) or proteins in an individual (the MAPKAP-2 genetic profile), alone or in conjunction with information on other genetic defects contributing to an inflammatory related disorder allows customization of therapy to the individual's genetic profile, the goal of “pharmacogenomics”. For example, an individual's MAPKAP-2 genetic profile, can enable a doctor to: 1) more effectively prescribe a drug that will address the underlying MAPKAP-2 associated disorder; and 2) better determine the appropriate dosage of a particular drug for the particular individual.

For example, the expression level of MAPKAP-2 kinase proteins, alone or in conjunction with the expression level of other genes, known to contribute to the same disease, can be measured in many patients at various stages of the disease to generate a transcriptional or expression profile of the disease. Expression patterns of individual patients can then be compared to the expression profile of the disease to determine the appropriate drug and dose to administer to the patient.

The ability to target populations expected to show the highest clinical benefit, based on the MAPKAP-2 or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of MAPKAP-2 as a marker is useful for optimizing effective dose).

Consequently, an aspect of the present invention relates to diagnostic assays for determining MAPKAP-2 kinase and/or nucleic acid expression as well as MAPKAP-2 activity, in the context of a biological sample (e.g., blood, serum, cells, tissue) to thereby determine whether an individual is afflicted with a disease or disorder, or is at risk of developing a disorder, associated with aberrant MAPKAP-2 expression or activity.

The invention further provides for prognostic (or predictive) assays for determining whether an individual is at risk of developing a disorder associated with MAPKAP-2 kinase, nucleic acid expression or activity. For example, mutations in a MAPKAP-2 gene can be assayed in a biological sample. Such assays can be used for prognostic or predictive purpose to thereby prophylactically treat an individual prior to the onset of a disorder characterized by or associated with MAPKAP-2 kinase, nucleic acid expression or activity. Another aspect of the invention pertains to monitoring the influence of agents (e.g., drugs, compounds) on the expression or activity of MAPKAP-2 in clinical trials.

These and other agents are described in further detail in the following sections.

(i) Diagnostic Assays

The present invention provides means for determining if a subject has (diagnostic) or is at risk of developing (prognostic) a disease, condition or disorder that is associated with an aberrant MAPKAP-2 activity, e.g., an aberrant level of MAPKAP-2 protein or an aberrant bioactivity, such as results in the development of an immune related inflammatory disorder.

Accordingly, an aspect of the invention provides methods for determining whether a subject has or is likely to develop an immune related disorder, comprising determining the level of an MAPKAP-2 gene or protein, an MAPKAP-2 bioactivity and/or the presence of a mutation or particular polymorphic variant in the MAPKAP-2 gene.

In one embodiment, the method comprises determining whether a subject has an abnormal mRNA and/or protein level of MAPKAP-2, such as by Northern blot analysis, reverse transcription-polymerase chain reaction (RT-PCR), in situ hybridization, immunoprecipitation, Western blot hybridization, or immunohistochemistry.

According to the method, cells are obtained from a subject and the MAPKAP-2 protein or mRNA level is determined and compared to the level of MAPKAP-2 protein or mRNA level in a healthy subject. An abnormal level of MAPKAP-2 kinase or mRNA level is likely to be indicative of an aberrant MAPKAP-2 activity.

In another embodiment, the method comprises measuring at least one activity of MAPKAP-2. For example, regulation of the expression of MAPKAP-2 gene can be determined, e.g., as described herein. Comparison of the results obtained with results from similar analysis performed on MAPKAP-2 proteins from healthy subjects is indicative of whether a subject has an abnormal MAPKAP-2 activity.

Another embodiment of the invention provides for a method for detecting the presence or absence of MAPKAP-2 kinase or nucleic acid in a biological sample entails obtaining a biological sample from a test subject and contacting the biological sample with a compound or an agent capable of detecting MAPKAP-2 kinase or nucleic acid (e.g., mRNA, genomic DNA) that encodes a MAPKAP-2 kinase such that the presence of MAPKAP-2 kinase or nucleic acid is detected in the biological sample. Preferably, the agent used for detecting MAPKAP-2 mRNA or genomic DNA is a labeled nucleic acid probe capable of hybridizing to any MAPKAP-2 mRNA or genomic DNA that may be present in the sample. The nucleic acid probe can be, for example, a human MAPKAP-2 nucleic acid, such as the nucleic acid of SEQ ID NO:1 or a portion thereof, preferably of a length sufficient to specifically hybridize under stringent conditions to any MAPKAP-2 mRNA or genomic DNA suspected of being present in the sample. Other suitable probes for use in the diagnostic assays of the invention are described herein.

Alternatively, the agent may be a labeled probe such as an antibody that is specific for the gene product of SEQ ID NO:1—that is human MAPKAP-2 kinase. The antibody can be polyclonal, or more preferably, monoclonal. An intact antibody, or a fragment thereof (e.g., Fab or F(ab′).sub.2) can also be used. As noted supra, the term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled.

Examples of indirect labeling include detection of a primary antibody using a fluorescently labeled secondary antibody and end labeling of a DNA probe with biotin such that it can be detected with fluorescently labeled streptavidin.

The term “biological sample” is intended to include tissues, cells and biological fluids isolated from a subject, as well as tissues, cells and fluids present within a subject. That is, the detection method of the invention can be used to detect MAPKAP-2 mRNA, protein, or genomic DNA in a biological sample in vitro as well as in vivo. In vitro techniques for detection of MAPKAP-2 mRNA include Northern hybridizations and in situ hybridizations. In vitro techniques for detecting the presence or absence of a MAPKAP-2 kinase or a fragment thereof in a sample include enzyme linked immunosorbent assays (ELISAs), Western blots, immunoprecipitations and immunofluorescence. Likewise, in vitro techniques for detecting MAPKAP-2 genomic DNA include Southern hybridizations.

Thus, an embodiment of the invention relates to a method of detecting the presence of MAPKAP-2 in a sample comprising a) contacting said sample with the above-described nucleic acid probe, under conditions such that hybridization occurs, and b) detecting the presence of said probe bound to said nucleic acid molecule. One skilled in the art would select the nucleic acid probe according to techniques known in the an as described above. Samples to be tested include but should not be limited to RNA samples of human tissue.

In another embodiment, the methods further involve obtaining a control biological sample from a control subject, contacting the control sample with a compound or agent capable of detecting MAPKAP-2 kinase, mRNA, or genomic DNA, such that the presence of MAPKAP-2 kinase, mRNA or genomic DNA is detected in the biological sample, and comparing the presence of MAPKAP-2 kinase, mRNA or genomic DNA in the control sample with the presence of MAPKAP-2 kinase, mRNA or genomic DNA in the test sample.

In vivo techniques for detecting MAPKAP-2 kinase include introducing into a subject a labeled anti-MAPKAP-2 kinase specific-antibody. For example, the antibody can be labeled with a radioactive marker whose presence and location in a subject can be detected by standard imaging techniques.

Kits for detecting the presence or absence of human MAPKAP-2 kinase in a biological sample are also provided. For example, the kit can comprise a labeled compound or agent capable of detecting MAPKAP-2 kinase or mRNA in a biological sample; means for determining the amount of MAPKAP-2 in the sample; and means for comparing the amount of MAPKAP-2 in the sample with a standard. The compound or agent can be packaged in a suitable container. The kit can further comprise instructions for using the kit to detect MAPKAP-2 kinase or nucleic acid.

(ii) Prognostic Assay

The diagnostic methods described herein can furthermore be utilized to identify subjects having or at risk of developing a disease or disorder associated with aberrant MAPKAP-2 expression or activity. For example, the assays described herein, such as the preceding diagnostic assays or the following assays, can be utilized to identify a subject having or at risk of developing a disorder associated with MAPKAP-2 kinase, nucleic acid expression or activity.

As a consequence, there is provided a method for identifying a disease or disorder associated with aberrant MAPKAP-2 kinase expression or activity in which a test sample is obtained from a subject and MAPKAP-2 kinase or nucleic acid (e.g., mRNA, genomic DNA) is detected, wherein the presence of MAPKAP-2 kinase or nucleic acid is diagnostic for a subject having or at risk of developing a disease or disorder associated with aberrant MAPKAP-2 expression or activity.

As used herein, a “test sample” refers to a biological sample obtained from a subject of interest. For example, a test sample can be a biological fluid (e.g., serum), cell sample, or tissue.

More, the prognostic assays described herein can also be used to determine whether a subject can be administered an agent (e.g., an agonist, antagonist, peptidomimetic, protein, peptide, nucleic acid, small molecule, or other drug candidate) to treat a disease or disorder associated with aberrant MAPKAP-2 expression or activity.

In accordance with the above, there are provided methods for determining whether a subject can be effectively treated with an agent for a disorder associated with aberrant MAPKAP-2 expression or activity. The method comprises obtaining a test sample from a subject suspected of being at risk of developing a pathological condition associated with aberant MAPKAP-2 activity or expression followed by detecting MAPKAP-2 kinase or nucleic acid expression or activity e.g., wherein the abundance of MAPKAP-2 kinase or nucleic acid expression or activity is diagnostic for the subject who, can, in turn, be administered the agent to treat a disorder associated with aberrant MAPKAP-2 expression or activity.

The methods of the invention as noted supra can also be used to detect genetic alterations in a MAPKAP-2 gene, thereby determining if a subject with the altered gene is at risk for a disorder associated with the MAPKAP-2 gene. In preferred embodiments, the methods include detecting, in a sample of cells from the subject, the presence or absence of a genetic alteration characterized by at least one of an alteration affecting the integrity of a gene encoding a MAPKAP-2-polypeptide, or the mis-expression of the MAPKAP-2 gene.

Such genetic alterations can be detected by ascertaining the existence of at least one of 1) a deletion of one or more nucleotides from a MAPKAP-2 encoding gene; 2) an addition of one or more nucleotides to a MAPKAP-2 gene; 3) a substitution of one or more nucleotides of a MAPKAP-2 gene, 4) a chromosomal rearrangement of a MAPKAP-2 gene; 5) an alteration in the level of a messenger RNA transcript of a MAPKAP-2 gene, 6) aberrant modification of a MAPKAP-2 gene, such as of the methylation pattern of the genomic DNA, 7) the presence of a non-wild type splicing pattern of a messenger RNA transcript of a MAPKAP-2 gene, 8) a non-wild type level of a MAPKAP-2-polypeptide, 9) allelic loss of a MAPKAP-2 gene, and 10) inappropriate post-translational modification of a MAPKAP-2-polypeptide.

Additional MAPKAP-2 related diseases or pathological condition's associated with its aberrant expression can be diagnosed by methods comprising determining from a sample derived from a subject an abnormally decreased or increased level of MAPKAP-2 expression. Decreased or increased expression can be measured at the RNA level using any of the methods well known in the art for the quantitation of polynucleotides, such as, for example, PCR, RT-PCR, RNase protection, Northern blotting and other hybridization methods. Assay techniques that can be used to determine levels of a polypeptide having a sequence as substantially set forth in SEQ ID NO:2 in a sample derived from a host are well known to those of skill in the art. Such assay methods include radioimmunoassays, competitive-binding assays, Western Blot analysis and ELISA assays.

In an exemplary embodiment, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the MAPKAP-2-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a MAPKAP-2 encoding gene under conditions such that hybridization and amplification of the MAPKAP-2-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

Mutations in a MAPKAP-2 gene from a sample cell can also be identified by alterations in restriction enzyme cleavage patterns. For example, sample and control DNA may be isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in MAPKAP-2 encoding gene can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759).

For example, genetic mutations in MAPKAP-2 can be identified in two-dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the MAPKAP-2 encoding gene and detect mutations by comparing the sequence of the sample MAPKAP-2 with the corresponding wild-type (control) sequence. Examples of sequencing reactions include those based on techniques developed by Maxam and Gilbert, Proc. Natl. Acad. Sci. USA 74:560 (1977) or Sanger, Proc. Natl. Acad. Sci. USA 74:5463(1977). It is also contemplated that any of a variety of automated sequencing procedures can be utilized when performing the diagnostic assays (Biotechniques 19:448, (1995), including sequencing by mass spectrometry (see, e.g., PCT International Publication No. WO 94/16101; Cohen et al. Adv. Chromatogr. 36:127-162(1996); and Griffin et al. Appl. Biochem. Biotechnol. 38:147-159 (1993)).

Other methods for detecting mutations in the MAPKAP-2 gene include methods in which protection from cleavage agents is used to detect mismatched bases in RNA/RNA or RNA/DNA heteroduplexes (Myers et al. Science 230:1242, (1995)). In general, the art technique of “mismatch cleavage” starts by providing heteroduplexes formed by hybridizing (labeled) RNA or DNA containing the wild-type MAPKAP-2 sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See; for example, Cotton et al. Proc. Natl. Acad. Sci USA 85:4397(1988); Saleeba et al. Methods Enzymol. 217:286-295(1992). In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in MAPKAP-2 cDNAs obtained from samples of cells. For example, the mutY enzyme of E. coli cleaves A at G/A mismatches and the thymidine DNA glycosylase from HeLa cells cleaves T at G/T mismatches (Hsu et al. Carcinogenesis 15:1657-1662 (1994)). According to an exemplary embodiment, a probe based on a MAPKAP-2 sequence, e.g., a wild-type MAPKAP-2 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility may be employed to identify mutations in MAPKAP-2 encoding genes.

Another method employs single strand conformation polymorphism (SSCP) to detect differences in electrophoretic mobility between mutant and wild type nucleic acids (Orita et al. Proc Natl Acad. Sci USA: 86:2766(1989), Hayashi Genet Anal Tech Appl 9:73-79 (1992)). Single-stranded DNA fragments of sample and control MAPKAP-2 nucleic acids are allowed to denature and then allowed to renature. The secondary structure of single-stranded nucleic acids varies according to sequence, the resulting alteration in electrophoretic mobility will enable one to detect even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al., Trends Genet 7:5, (1991)).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner, Biophys Chem 265:12753(1987)), U.S. Pat. No. 6,146,841.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, one may employ allele specific amplification technology which depends on selective PCR amplification in conjunction with the instant invention. Oligonucleotides used as primers for specific amplification may carry the mutation of interest in the center of the molecule (so that amplification depends on differential hybridization) (Gibbs et al. Nucleic Acids Res. 17:2437-2448 (1989)) or at the extreme 3′ end of one primer where, under appropriate conditions, mismatch can prevent, or reduce polymerase extension. See Prossner et al. Tibtech 11:238(1993). In addition it may be desirable to introduce a novel restriction site in the region of the mutation to create cleavage-based detection (Gasparini et al., Mol. Cell Probes 6:1 (1992)). It is anticipated that in certain embodiments amplification may also be performed using Taq ligase for amplification, see Barany, Proc. Natl. Acad. Sci USA 88:189 (1991). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a MAPKAP-2 gene.

Furthermore, any cell type or tissue in which MAPKAP-2 is expressed may be utilized in the prognostic assays described herein.

(iii) Antisense Molecules/Gene Therapy

Antisense Molecules

Agents which decrease the cellular level and/or the activity of the overexpressed and/or overactive MAPKAP-2 kinase or nucleic acid expression or activity will find use in various treatment regiments. Techniques for decreasing the cellular level and/or the activity MAPKAP-2 may include, but are not limited to antisense or ribozyme approaches, and/or gene therapy approaches, each of which is well known to those of skill in the art.

Provided herein are antisense oligonucleotides having a nucleotide sequence capable of binding specifically with any portion of an mRNA that encodes a MAPKAP-2 kinase of SEQ ID NO:2 so as to prevent translation of the mRNA. The antisense oligonucleotide may have a sequence capable of binding specifically with any portion of the sequence of the cDNA encoding the MAPKAP-2 kinase of the invention.

The cDNA sequence SEQ ID NO:1 provided herein, may be used in another embodiment of the invention to study the physiological relevance of the novel human signal-transduction kinase in cells, especially cells of hematopoietic origin, by knocking out the endogenous gene by use of anti-sense constructs.

Some methods of delivering the proposed antisense reagents include: 1) encapsulation in liposomes; 2) transduction by retroviral vectors; 3) localization to nuclear compartment utilizing nuclear targeting site found on most nuclear proteins; 4) transfection of cells ex vivo with subsequent re-implantation or administration of the transfected cells; and 5) a DNA transporter system.

Consequently, included in the scope of the invention are oligoribonucleotides, including antisense RNA and DNA molecules and ribozymes that function to inhibit translation of MAPKAP-2 encoding mRNA. Anti-sense RNA and DNA molecules act to directly block the translation of mRNA by binding to targeted mRNA and preventing protein translation. With respect to antisense DNA, oligodeoxyribonucleotides derived from the translation initiation site, e.g., between −10 and +10 regions of the relevant nucleotide sequence, are preferred.

Ribozymes are enzymatic RNA molecules capable of catalyzing the specific cleavage of RNA. The mechanism of ribozyme action involves sequence specific interaction of the ribozyme molecule to complementary target RNA, followed by a endonucleolytic cleavage. Within the scope of the invention are engineered hammerhead or other motif ribozyme molecules that specifically and efficiently catalyze endonucleolytic cleavage of RNA sequences encoding protein complex components.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate targets may also be evaluated by testing their accessibility to hybridization with complementary oligonucleotides, using ribonuclease protection assays. See, Draper, PCT WO 93/23569.

Both anti-sense RNA and DNA molecules and ribozymes of the invention may be prepared by any method known in the art for the synthesis of RNA molecules. See, Draper, id. hereby incorporated by reference herein. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules may be introduced as a means of increasing intracellular stability and half-life. Possible modifications include but are not limited to the addition of flanking sequences of ribo- or deoxynucleotides to the 5′ and/or 3′ ends of the molecule or the use of phosphorothioate or 2′ O-methyl rather than phosphodiesterase linkages within the oligodeoxyribonucleotide backbone.

Oligomers of 12-21 nucleotides are most preferred in the practice of the present invention, particularly oligomers of 12-18 nucleotides. Oligos are, in principle, effective for inhibiting translation of the transcript, and capable of inducing the effects herein described. Translation is most effectively inhibited by blocking the mRNA at a site at or near the initiation codon. Thus, oligonucleotides complementary to the 5′-terminal region of the human kinase mRNA transcript are preferred. Secondary or tertiary structure which might interfere with hybridization is minimal in this region. Moreover, sequences that are too distant in the 3′ direction from the initiation site can be less effective in hybridizing the mRNA transcripts because of a “read-through” phenomenon whereby the ribosome appears to unravel the antisense/sense duplex to permit translation of the message. Oligonucleotides which are complementary to and hybridizable with any portion of the novel human signal-transduction kinase mRNA are contemplated for therapeutic use.

U.S. Pat. No. 5,639,595, Identification of Novel Drugs and Reagents, issued Jun. 17, 1997, teaches methods of identifying oligonucleotide sequences that display in vivo activity. Expression vectors containing random oligonucleotide sequences derived from previously known polynucleotides are transformed/transfected into cells. The cells are then assayed for a phenotype resulting from the desired activity of the oligonucleotide. Once cells with the desired phenotype have been identified, the sequence of the oligonucleotide having the desired activity can be identified. Identification may be accomplished by recovering the vector or by polymerase chain reaction (PCR) amplification and sequencing the region containing the inserted nucleic acid material.

Nucleotide sequences that are complementary to the novel signal-transduction kinase polypeptide encoding polynucleotide sequence described can be synthesized for antisense therapy. These antisense molecules may be DNA, stable derivatives of DNA such as phosphorothioates or methylphosphonates, RNA, stable derivatives of RNA such as 2′-O-alkyl RNA, or other oligonucleotide mimetics. U.S. Pat. No. 5,652,355, Hybrid Oligonucleotide Phosphorothioates, issued Jul. 29, 1997, and U.S. Pat. No. 5,652,356, Inverted Chimeric and Hybrid Oligonucleotides, issued Jul. 29, 1997, which describe the synthesis and effect of physiologically-stable antisense molecules, are incorporated by reference. Signal-transduction kinase antisense molecules may be introduced into cells by microinjection, liposome encapsulation or by expression from vectors harboring the antisense sequence. Antisense therapy may be particularly useful for the treatment of diseases where it is beneficial to reduce the signal-transduction kinase activity.

Gene Therapy

The human signal-transduction kinase described herein may administered to a subject via gene therapy. Moreover, the invention polypeptide may be delivered to the cells of target organs in this manner. Conversely, signal-transduction kinase antisense gene therapy may be used to reduce the expression of the polypeptide in the cells of target organs. See Miller, Nature 357:455-460, (1992). According to Miller, advances have resulted in practical approaches to human gene therapy that have demonstrated positive initial results.

In its simplest form, gene transfer can be performed by simply injecting minute amounts of DNA into the nucleus of a cell, through a process of microinjection. Capecchi, M R, Cell 22:479-88 (1980). Once recombinant genes are introduced into a cell, they can be recognized by the cells normal mechanisms for transcription and translation, and a gene product will be expressed.

Other methods have also been attempted for introducing DNA into larger numbers of cells. These methods include: transfection, wherein DNA is precipitated with CaPO₄ and taken into cells by pinocytosis (Chen C. and Okayama H, Mol. Cell Biol. 7:2745-52 (1987)); electroporation, wherein cells are exposed to large voltage pulses to introduce holes into the membrane (Chu G. et al., Nucleic Acids Res., 15:1311-26 (1987)); lipofection/liposome fusion, wherein DNA is packaged into lipophilic vesicles which fuse with a target cell (Felgner P L., et al., Proc. Natl. Acad. Sci. USA. 84:7413-7 (1987)); and particle bombardment using DNA bound to small projectiles (Yang N S. et al., Proc. Natl. Acad. Sci. 87:9568-72 (1990)). Another method for introducing DNA into cells is to couple the DNA to chemically modified proteins.

It has also been shown that adenovirus proteins are capable of destabilizing endosomes and enhancing the uptake of DNA into cells. The admixture of adenovirus to solutions containing DNA complexes, or the binding of DNA to polylysine covalently attached to adenovirus using protein crosslinking agents substantially improves the uptake and expression of the recombinant gene. Curiel D T, et al., Am. J. Respir. Cell. Mol. Biol., 6:247-52 (1992).

As used herein “gene transfer” means the process of introducing a foreign nucleic acid molecule into a cell. Gene transfer is commonly performed to enable the expression of a particular product encoded by the gene. The product may include a protein, polypeptide, anti-sense DNA or RNA, or enzymatically active RNA. Gene transfer can be performed in cultured cells or by direct administration into animals. Generally gene transfer involves the process of nucleic acid contact with a target cell by non-specific or receptor mediated interactions, uptake of nucleic acid into the cell through the membrane or by endocytosis, and release of nucleic acid into the cytoplasm from the plasma membrane or endosome. Expression may require, in addition, movement of the nucleic acid into the nucleus of the cell and binding to appropriate nuclear factors for transcription.

As used herein “gene therapy” is a form of gene transfer and is included within the definition of gene transfer as used herein and specifically refers to gene transfer to express a therapeutic product from a cell in vivo or in vitro. Gene transfer can be performed ex vivo on cells which are then transplanted into a patient, or can be performed by direct administration of the nucleic acid or nucleic acid-protein complex into the patient.

The MAPKAP-2 kinase-coding region can be ligated into viral vectors which mediate transfer of the kinase polypeptide DNA by infection of recipient host cells. Suitable viral vectors include retrovirus, adenovirus, adeno-associated virus, herpes virus, vaccinia virus, polio virus and the like. See, e.g., U.S. Pat. No. 5,624,820, Episomal Expression Vector for Human Gene Therapy, issued Apr. 29, 1997. Nucleic acid coding regions of the present invention are incorporated into effective eukaryotic expression vectors, which are directly administered or introduced into somatic cells for gene therapy (a nucleic acid fragment comprising a coding region, preferably mRNA transcripts, may also be administered directly or introduced into somatic cells). See, e.g., U.S. Pat. No. 5,589,466, issued Dec. 31, 1996. Such nucleic acids and vectors may remain episomal or may be incorporated into the host chromosomal DNA as a provirus or portion thereof that includes the gene fusion and appropriate eukaryotic transcription and translation signals, i.e., an effectively positioned RNA polymerase promoter 5′ to the transcriptional start site and ATG translation initiation codon of the gene fusion as well as termination codon(s) and transcript polyadenylation signals effectively positioned 3′ to the coding region.

Alternatively, the invention polypeptide encoding DNA can be transferred into cells for gene therapy by non-viral techniques including receptor-mediated targeted DNA transfer using ligand-DNA conjugates or adenovirus-ligand-DNA conjugates, lipofection membrane fusion or direct microinjection. These procedures and variations thereof are suitable for ex vivo, as well as in vivo human signal-transduction kinase polypeptide gene therapy according to established methods in this art. See Chowdhury et al, Science 254:1802-1805, 1991 and Wilson, Hum. Gene Ther. 3:179-222, 1992) ex vivo approaches, which can be modified to transfer the MAPKAP-2 nucleic acid sequence disclosed herein and re-implanted into an animal.

Other nonviral techniques for the delivery of a MAPKAP-2 nucleic acid sequence into a cell can be used, including direct naked DNA uptake (e.g., Wolff et al., Science 247: 1465-1468, (1990)), receptor-mediated DNA uptake, e.g., using DNA coupled to asialoorosomucoid which is taken up by the asialoglycoprotein receptor in the liver (Wu and Wu, J. Biol. Chem. 262:4429-4432, (1987 and liposome-mediated delivery (Kaneda et al., Expt. Cell Res. 173:56-69, (1987) and Kaneda et al., Science 243:375-378, (1989). Many of these physical methods can be combined with one another and with viral techniques; enhancement of receptor-mediated DNA uptake can be effected, for example, by combining its use with adenovirus (Curiel et al., Proc. Natl. Acad. Sci. USA 88:8850-8854, (1991).

The MAPKAP-2 kinase or nucleic acid encoding the MAPKAP-2 kinase may also be administered via an implanted device that provides a support for growing cells. Thus, the cells may remain in the implanted device and still provide the useful and therapeutic agents of the present invention.

In an exemplary embodiment, an expression vector containing the MAPKAP-2 coding sequence (SEQ ID NO:1) is inserted into cells, the cells are grown in vitro and then infused in large numbers into patients. In another preferred embodiment, a DNA segment containing a promoter of choice (for example a strong promoter) is transferred into cells containing an endogenous MAPKAP-2 gene in such a manner that the promoter segment enhances expression of the endogenous MAPKAP-2 gene (for example, the promoter segment is transferred to the cell such that it becomes directly linked to the endogenous MAPKAP-2 gene).

Target cell populations (e.g., cells of the immune system) may be modified by introducing altered forms of MAPKAP-2 kinase in order to modulate the activity of such cells.

In another preferred embodiment, a method of gene replacement is set forth. “Gene replacement” as used herein means supplying a nucleic acid sequence which is capable of being expressed in vivo in an animal and thereby providing or augmenting the function of an endogenous gene which is missing or defective in the animal.

PCR Diagnostics

The nucleic acid sequence, oligonucleotides, fragments, portions or antisense molecules thereof, may be used in diagnostic assays of body fluids or biopsied tissues to detect the expression level of the novel human signal-transduction kinase molecule. For example, sequences designed from the cDNA sequence SEQ ID NO:1 or sequences comprised in SEQ ID NO:2 can be used to detect the presence of the mRNA transcripts in a patient or to monitor the modulation of transcripts during treatment.

One method for amplification of target nucleic acids, or for later analysis by hybridization assays, is known as the polymerase chain reaction (“PCR”) or PCR technique. The PCR technique can be applied to detect sequences of the invention in suspected samples using oligonucleotide primers spaced apart from each other and based on the genetic sequence, e.g., SEQ ID NO:1, set forth herein. The primers are complementary to opposite strands of a double stranded DNA molecule and are typically separated by from about 50 to 450 nucleotides or more (usually not more than 2000 nucleotides). This method entails preparing the specific oligonucleotide primers followed by repeated cycles of target DNA denaturation, primer binding, and extension with a DNA polymerase to obtain DNA fragments of the expected length based on the primer spacing. The degree of amplification of a target sequence is controlled by the number of cycles that are performed and is theoretically calculated by the simple formula 2n where n is the number of cycles. See, e.g., Perkin Elmer, PCR Bibliography, Roche Molecular Systems, Branchburg, N.J.; CLONTECH products, Palo Alto, Calif.; U.S. Pat. No. 5,629,158, Solid Phase Diagnosis of Medical Conditions, issued May 13, 1997.

Monitoring of Effects During Clinical Trials

The ability to target populations expected to show the highest clinical benefit, based on the MAPKAP-2 or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of MAPKAP-2 as a marker is useful for optimizing effective dose).

The treatment of an individual with an MAPKAP-2 therapeutic can be monitored by determining MAPKAP-2 characteristics, such as MAPKAP-2 protein level or activity, MAPKAP-2 mRNA level, and/or MAPKAP-2 transcriptional level. This measurements will indicate whether the treatment is effective or whether it should be adjusted or optimized. Thus, MAPKAP-2 can be used as a marker for the efficacy of a drug during clinical trials.

Monitoring the influence of agents (e.g., drugs or compounds) on the expression or activity of a MAPKAP-2 kinase are also contemplated by the invention and can be applied not only in basic drug screening, but also in clinical trials. For example, the effectiveness of an agent determined by a screening assay as described above to increase MAPKAP-2 gene expression, protein levels, or upregulate MAPKAP-2 activity, can be monitored in clinical trials of subjects exhibiting decreased MAPKAP-2 gene expression, polypeptide levels, or down-regulated MAPKAP-2 activity. Alternatively, the effectiveness of an agent determined by a screening assay to decrease MAPKAP-2 gene expression, polypeptide levels, or downregulate MAPKAP-2 activity, can be monitored in clinical trials of subjects exhibiting increased MAPKAP-2 gene expression, polypeptide levels, or upregulated MAPKAP-2 activity. In such clinical trials, the expression or activity of a MAPKAP-2 gene, and preferably, other genes that have been implicated in a disorder can be used as a “read out” or markers of the phenotype of a particular cell.

For example, and not by way of limitation, genes, including MAPKAP-2, that are modulated in cells by treatment with an agent (e.g., compound, drug or small molecule) which modulates MAPKAP-2 activity (e.g., identified in a screening assay as described herein) can be identified. Thus, to study the effect of agents on a MAPKAP-2 associated disorder, for example, in a clinical trial, cells can be isolated and RNA prepared and analyzed for the levels of expression of MAPKAP-2 and other genes implicated in the MAPKAP-2 associated disorder, respectively. The levels of gene expression (i.e., a gene expression pattern) can be quantified by Northern blot analysis or RT-PCR, as described herein, or alternatively by measuring the amount of polypeptide produced, by one of the methods as described herein, or by measuring the levels of activity of MAPKAP-2 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a MAPKAP-2 kinase, mRNA, or genomic DNA in the pre-administration sample; (iii) obtaining one or more post-administration samples from the subject; (iv) detecting the level of expression or activity of the MAPKAP-2 kinase, mRNA, or genomic DNA in the post-administration samples; (v) comparing the level of expression or activity of the MAPKAP-2 kinase, mRNA, or genomic DNA in the pre-administration sample with the MAPKAP-2 kinase, mRNA, or genomic DNA in the post administration sample or samples; and (vi) altering the administration of the agent to the subject accordingly.

For example, increased administration of the agent may be desirable to increase the expression or activity of MAPKAP-2 to higher levels than detected, i.e., to increase the effectiveness of the agent. Alternatively, decreased administration of the agent may be desirable to decrease expression or activity of MAPKAP-2 to lower levels than detected, i.e. to decrease the effectiveness of the agent. According to such an embodiment, MAPKAP-2 expression or activity may be used as an indicator of the effectiveness of an agent, even in the absence of an observable phenotypic response.

Cells of a subject may also be obtained before and after administration of an MAPKAP-2 therapeutic to detect the level of expression of genes other than MAPKAP-2, to verify that the MAPKAP-2 therapeutic does not increase or decrease the expression of genes which could be deleterious. This can be done, e.g., by using the method of transcriptional profiling. Thus, mRNA from cells exposed in vivo to an MAPKAP-2 therapeutic and mRNA from the same type of cells that were not exposed to the MAPKAP-2 therapeutic could be reverse transcribed and hybridized to a chip containing DNA from numerous genes, to thereby compare the expression of genes in cells treated and not treated with an MAPKAP-2-therapeutic. If, for example an MAPKAP-2 therapeutic turns on the expression of a proto-oncogene in an individual, use of this particular MAPKAP-2 therapeutic may be undesirable.

To reiterate, biological tissue may be repeatedly assayed over time and the levels of the desired parameters, e.g., MAPKAP-2 kinase or mRNA or DNA be recorded over time to assess the usefulness of a therapeutic regime or determine the usefulness of a treatment protocol.

An embodiment of the invention pertains to a method for following progress of a therapeutic regime designed to alleviate a condition characterized by aberrant MAPKAP-2 kinase expression comprising:

(a) assaying a sample from a subject to determine level of a parameter selected from the group consisting of (i) a polypeptide encoded by a the nucleotide sequence of SEQ. ID. NO. 1 and (ii) a polypeptide encoded by the degenerate sequence thereto, (iii) a polypeptide having the amino acid sequence as set forth in SEQ ID NO: 2;

(b) assaying level of the parameter selected in (a) at a second time point and

(c) comparing said level at said second time point to the level determined in (a) as a determination of effect of said therapeutic regime.

A method for determining regression, progression or onset of a pathological disorder characterized by a dysfunctional signal transduction comprising incubating a sample obtained from a patient with said disorder with a complimentary nucleic acid hybridization probe having a sequence of nucleotides that are substantially homologous to those of SEQ. ID. NO. 1 and determining binding between the probe and any complimentary mRNA that may be present in said sample as determinative of the regression, progression or onset of the pathological disorder in the patient.

A method for determining regression, progression or onset of a pathological disorder characterized by a dysfunctional signal transduction comprising: contacting a sample, from a patient with the disorder, with a detectable probe that is specific for the gene product of the isolated nucleic acid molecule having a sequence of nucleotides as set forth in SEQ ID NO:1, under conditions favoring formation of a probe/gene product complex, the presence of which is indicative of the regression progression or onset of the pathological disorder in the patient.

C. Methods of Treatment

The present invention also provides for both prophylactic and therapeutic methods of treating a subject at risk of (or susceptible to) a disorder or having a disorder associated with aberrant MAPKAP-2 expression or activity. With regards to both prophylactic and therapeutic methods of treatment, such treatments may be specifically tailored or modified, based on knowledge obtained from the field of pharmacogenomics.

The term “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.)

In accordance with the above, there are provided methods for tailoring an individual's prophylactic or therapeutic treatment with either the MAPKAP-2 kinase(s) of the present invention or MAPKAP-2 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

(i) Prophylactic Methods

In one aspect, the invention provides a method for preventing in a subject, a disease or condition associated with an aberrant MAPKAP-2 expression or activity, by administering to the subject a MAPKAP-2 kinase (SEQ ID NO:2) or an agent which modulates MAPKAP-2 expression or at least one MAPKAP-2 activity. Subjects at risk for a disease which is caused or contributed to by aberrant MAPKAP-2 expression or activity can be identified by, for example, any or a combination of diagnostic or prognostic assays as described herein. Administration of a prophylactic agent can occur prior to the manifestation of symptoms characteristic of the MAPKAP-2 aberrancy, such that a disease or disorder is prevented or, alternatively, delayed in its progression. Depending on the type of MAPKAP-2 aberrancy, for example, a MAPKAP-2, MAPKAP-2 agonist or MAPKAP-2 antagonist agent can be used for treating the subject. The appropriate agent can be determined based on screening assays described herein.

(ii) Therapeutic Methods

Another aspect of the invention pertains to methods of modulating MAPKAP-2 expression or activity for therapeutic purposes. As a consequence, an exemplary embodiment teaches a modulatory method which involves contacting a cell with a MAPKAP-2 or agent that modulates one or more of the activities of MAPKAP-2 kinase activity associated with the cell. An agent that modulates MAPKAP-2 kinase activity can be an agent as described herein, such as a nucleic acid or a polypeptide, a naturally-occurring target molecule of a MAPKAP-2 kinase (e.g., a MAPKAP-2 phosphorylation substrate—Hsp-27 or another native substrate), a MAPKAP-2 antibody, a MAPKAP-2 agonist or antagonist, a peptidomimetic of a MAPKAP-2 agonist or antagonist, or other small molecule.

An exemplary embodiment is drawn to an agent that stimulates one or more MAPKAP-2 activities. Examples of such stimulatory agents include active MAPKAP-2 kinase and a nucleic acid molecule encoding MAPKAP-2 kinase having a sequence substantially as set forth in SEQ ID NO:2 that has been introduced into the cell.

In another embodiment, the agent inhibits one or more MAPKAP-2 activities.

Examples of such inhibitory agents include antisense MAPKAP-2 nucleic acid molecules, anti-MAPKAP-2 antibodies, and MAPKAP-2 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a MAPKAP-2 kinase or nucleic acid molecule.

An exemplary method of the present invention involves administering an agent (e.g., an agent identified by a screening assay described herein), or combination of agents that modulates (e.g., upregulates or downregulates) MAPKAP-2 expression or activity. In another embodiment, the method involves administering a MAPKAP-2 kinase or nucleic acid molecule as therapy to compensate for reduced or aberrant MAPKAP-2 expression or activity.

Stimulation of MAPKAP-2 activity is desirable in situations in which MAPKAP-2 is abnormally downregulated and/or in which increased MAPKAP-2 activity is likely to have a beneficial effect. For example, stimulation of MAPKAP-2 activity is desirable in situations in which a MAPKAP-2 is downregulated and/or in which increased MAPKAP-2 activity is likely to have a beneficial effect. Likewise, inhibition of MAPKAP-2 activity is desirable in situations in which MAPKAP-2 is abnormally upregulated and/or in which decreased MAPKAP-2 activity is likely to have a beneficial effect.

(iii) Pharmacogenomics

Pharmacogenomics deals with clinically significant hereditary variations in the response to drugs due to altered drug disposition and abnormal action in affected persons. See, for example, Eichelbaum, M. et al. Clin. Exp. Pharmacol. Physiol. 23(10-11):983-985 (1996) and Linder, M. W. et al. Clin. Chem. 43(2):254-266 (1997). In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms.

Knowledge of the particular alteration or alterations, resulting in defective or deficient MAPKAP-2 genes or proteins in an individual (the MAPKAP-2 genetic profile), alone or in conjunction with information on other genetic defects contributing to the same disease (the genetic profile of the particular disease) allows a customization of the therapy for a particular disease to the individual's genetic profile, the goal of “pharmacogenomics”.

For example, subjects having a specific allele of a MAPKAP-2 gene may or may not exhibit symptoms of a particular disease or be predisposed of developing symptoms of a particular disease. Further, if those subjects are symptomatic, they may or may not respond to a certain drug, e.g., a specific MAPKAP-2 therapeutic, but may respond to another. Thus, generation of an MAPKAP-2 genetic profile, (e.g., categorization of alterations in MAPKAP-2 genes which are associated with the development of rheumatoid arthritis, for example), from a population of subjects, who are symptomatic for a disease or condition that is caused by or contributed to by a defective and/or deficient MAPKAP-2 gene and/or protein (a MAPKAP-2 genetic population profile) and comparison of an individual's MAPKAP-2 profile to the population profile, permits the selection or design of drugs that are expected to be safe and efficacious for a particular patient or patient population (i.e., a group of patients having the same genetic alteration).

As an example, a MAPKAP-2 population profile can be performed, by determining the MAPKAP-2 profile, e.g., the identity of MAPKAP-2 genes, in a patient population having a disease, which is caused by or contributed to by a defective or deficient MAPKAP-2 gene. Optionally, the MAPKAP-2 population profile can further include information relating to the response of the population to a MAPKAP-2 therapeutic, using any of a variety of methods, including, monitoring: 1) the severity of symptoms associated with the MAPKAP-2 related disease, 2) MAPKAP-2 gene expression level, 3) MAPKAP-2 mRNA level, and/or 4) MAPKAP-2 protein level and (iii) dividing or categorizing the population based on the particular genetic alteration or alterations present in its MAPKAP-2 gene or a MAPKA-2 pathway gene. The MAPKAP-2 genetic population profile can also, optionally, indicate those particular alterations in which the patient was either responsive or non-responsive to a particular therapeutic. This information or population profile, is then useful for predicting which individuals should respond to particular drugs, based on their individual MAPKAP-2 profile.

In a preferred embodiment, the MAPKAP-2 profile is a transcriptional or expression level profile and step (i) is comprised of determining the expression level of MAPKAP-2 proteins, alone or in conjunction with the expression level of other genes, known to contribute to the same disease. The MAPKAP-2 profile can be measured in many patients at various stages of the disease.

Pharmacogenomic studies can also be performed using transgenic animals. For example, one can produce transgenic mice, e.g., as described herein, which contain a specific allelic variant of an MAPKAP-2 gene. These mice can be created, e.g., by replacing their wild-type MAPKAP-2 gene with an allele of the human MAPKAP-2 gene. The response of these mice to specific MAPKAP-2 therapeutics can then be determined.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect.

Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict a drug response. According to this method, if a gene that encodes a drug target is known (e.g., a MAPKAP-2 kinase of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

On the other hand, a method termed the “gene expression profiling”, can be utilized to identify genes that predict drug response. For example, the gene expression of an animal dosed with a drug (e.g., a MAPKAP-2 kinases or MAPKAP-2 kinase modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. It is believed that the knowledge gleaned from the above, when applied to dosing or drug selection, can avoid adverse reactions or therapeutic failure and thus enhance therapeutic or prophylactic efficiency when treating a subject with a MAPKAP-2 kinase or MAPKAP-2 modulator, such as a modulator identified by one of the exemplary screening assays described herein.

The MAPKAP-2 kinase of the present invention, as well as agents, or modulators which have a stimulatory or inhibitory effect on MAPKAP-2 activity (e.g., MAPKAP-2 gene expression) as identified by a screening assay described herein above can be administered to individuals to treat (prophylactically or therapeutically) disorders (e.g., cardiovascular disorders such as congestive heart failure) associated with aberrant MAPKAP-2 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a MAPKAP-2 molecule or MAPKAP-2 modulator as well as tailoring the dosage and/or therapeutic regimen of treatment with a MAPKAP-2 molecule or MAPKAP-2 modulator.

VI. Zinc Finger Containing Proteins

The paradigm that the primary mechanism for governing the expression of genes involves protein switches that bind DNA in a sequence specific manner was established in 1967—Ptashne, M., Nature (London) 214, 323-4 (1967). Diverse structural families of DNA binding proteins have been described.

The selective expression of any one gene is accomplished primarily through the interaction of protein transcription factors with characteristic nucleotide sequences located in the control regions of the gene, which are most commonly located near to, or upstream from, the actual coding region. The binding of a set of such factors, or regulatory proteins, acts as a molecular switch for the activation of the RNA polymerase and other components of the transcriptional machinery, which are common to all genes. The supply of a particular combination of such transcription factors ensures that a gene is switched on at the right place and at the right time. Thus, transcriptional regulation is primarily achieved by the sequence-specific binding of proteins to DNA and RNA.

Of the known protein motifs involved in the sequence specific recognition of DNA, the zinc finger protein is unique in its modular nature. To date, more than two hundred proteins, many of them transcription factors, have been shown to possess zinc fingers domains. The CYS.₂-His₂ zinc finger motif, identified first in the DNA and RNA binding transcription factor TFIIIA (Miller, J., McLachlan, A. D. & Klug, A., Embo J 4, 1609-14 (1985), is perhaps the ideal structural scaffold on which a sequence specific protein might be constructed. Zinc fingers connect transcription factors to their target genes mainly by binding to specific sequences of DNA base pairs—the “rungs” in the DNA “ladder”. In fact, the Cys.₂-His₂ zinc finger motif constitutes the most frequently utilized nucleic acid binding motif in eukaryotes.

These motifs can be used as modular building blocks for the construction of larger protein domains that recognize and bind to specific DNA sequences. Detailed models for the interaction of zinc fingers and DNA have been proposed (Berg, 1988; and Churchill, et al., 1990). Zinc finger proteins have also been reported which bind to RNA—Clemens, et al., Science, 260:530, (1993).

Importantly, control of gene expression using designed zinc finger peptides has been demonstrated by the specific inhibition of an oncogene mouse cell line and also by switching on genes in expression plasmids. These experiments have shown zinc finger DNA-binding domains can be engineered de novo to recognize given DNA sequences. It is understood that five to six individual zinc fingers linked together would recognise a DNA sequence 15-18 bp in length, sufficiently long to constitute a rare address in the human genome. By adding functional groups to the engineered DNA-binding domains, e.g. silencing domains, novel transcription factors can be generated to up- or downregulate expression of a target gene. Among potential applications are the repression of oncogene expression and the disruption of the reproductive cycle of virus infection. Klug, A, J Mol 293(2):215-8 (1999).

U.S. Pat. No. 6,140,466 teaches methods for designing new zinc finger modules that bind to a cellular nucleotide sequence and modulate the function of the cellular nucleotide sequence. The proposed new variant binds to either DNA or RNA and may enhance or suppress transcription from a promoter or from within a transcribed region of a structural gene. The cellular nucleotide sequence may be a sequence which is a naturally occurring sequence in the cell, or it may be a viral-derived nucleotide sequence in the cell. See also U.S. Pat. No. 5,789,538.

As a consequence, the herein disclosed novel nucleotide sequences can used to design now zinc finger containing proteins that can be used to express endogenous MAPKAP-2 from a cell line without the need to transform cells with heterologous DNA encoding MAPKAP-2 kinase. The proposed new zinc finger proteins will be specific for the polynucleotide sequences disclosed herein or complementary sequences thereof, which encode substantially the polypeptide of SEQ ID NO:2. The resulting proteins can, in turn, be used to treat a MAPKAP-2 related disorder.

VII. Pharmaceutical Formulations and Modes of Administration

Pharmaceutically useful compositions comprising the novel human kinase encoding DNA, human kinase encoding RNA, antisense sequences, or the human kinase having a sequence substantially as set forth in SEQ ID NO: 2, or variants and analogs which have the human kinase activity or otherwise modulate its activity, may be formulated according to known methods such as by the admixture of a pharmaceutically acceptable carrier. Examples of such carriers and methods of formulation may be found in Remington's Pharmaceutical Sciences (Maack Publishing Co, Easton, Pa.). To form a pharmaceutically acceptable composition suitable for effective administration, such compositions will contain an effective amount of the protein, DNA, RNA, or modulator.

The therapeutically effective dose refers to compositions of the invention that are administered to an individual in amounts sufficient to treat or diagnose human signal-transduction kinase polypeptide related disorders. The effective amount may vary according to a variety of factors such as the individual's condition, weight, sex and age. Other factors include the mode of administration. An effective but non-toxic amount of the compound desired can be employed as a signal-transduction kinase modulating agent.

Compounds identified according to the methods disclosed herein may be used alone at appropriate dosages defined by routine testing in order to obtain optimal modulation of a signal-transduction kinase, or its activity while minimizing any potential toxicity. In addition, co-administration or sequential administration of other agents may be desirable.

Toxicity and therapeutic efficacy of such compounds can be determined by standard pharmaceutical procedures in cell cultures or experimental animals, e.g., for determining the LD.sub.50. (the dose lethal to 50% of the population) and the ED.sub.50 (the dose therapeutically effective in 50% of the population). The dose ratio between toxic and therapeutic effects is the therapeutic index and it can be expressed as the ratio LD.sub.50/ED.sub.50. Compounds which exhibit large therapeutic indices are preferred. The data obtained from these cell culture assays and animal studies can be used in formulating a range of dosage for use in human. The dosage of such compounds lies preferably within a range of circulating concentrations that include the ED.sub.50 with little or no toxicity. The dosage may vary within this range depending upon the dosage form employed and the route of administration utilized.

The exact formulation, route of administration and dosage can be chosen by the individual physician in view of the patient's condition. See e.g. Fingl et al., in The Pharmacological Basis of Therapeutics, 1975, Ch. 1 p. 1. It should be noted that the attending physician would know how to and when to terminate, interrupt, or adjust administration due to toxicity, or to organ dysfunction. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine. The dosage should not be so large as to cause adverse side effects, such as unwanted cross-reactions, anaphylactic reactions, and the like.

Generally, the dosage will vary with the age, condition, sex and extent of disease in the patient, counter indications, if any, and other such variables, to be adjusted by the individual physician. Dosage can vary from 0.001 mg/kg to 50 mg/kg, preferably 0.1 mg/kg to 1.0 mg/kg, of the agonist or antagonist of the invention, in one or more administrations daily, for one or several days.

For oral administration, the compositions are preferably provided in the form of scored or unscored tablets containing 0.01, 0.05, 0.1, 0.5, 1.0, 2.5, 5.0, 10.0, 15.0, 25.0, and 50.0 milligrams of the active ingredient for the symptomatic adjustment of the dosage to the patient to be treated. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day. Even more particularly, the range varies from about 0.05 to about 1 mg/kg. Of course the dosage level will vary depending upon the potency of the particular compound. Certain compounds will be more potent than others. In addition, the dosage level will vary depending upon the bioavalability of the compound. The more bioavailable and potent the compound, the less compound will need to be administered through any delivery route, including but not limited to oral delivery. The dosages of the human signal-transduction kinase modulators are adjusted when combined to achieve desired effects. On the other hand, dosages of these various agents may be independently optimized and combined to achieve a synergistic result wherein the pathology is reduced more than it would be if either agent were used alone. Those skilled in the art will employ different formulations for nucleotides than for proteins or their inhibitors. Similarly, delivery of polynucleotides or polypeptides will be specific to particular cells and conditions.

For example, a dose can be formulated in animal models to achieve a circulating plasma concentration range that includes the IC.sub.50 as determined in cell culture (i.e., the concentration of the test compound which achieves a half-maximal disruption of the polypeptide complex, or a half-maximal inhibition of the cellular level and/or activity of a complex component). Such information can be used to more accurately determine useful doses in humans. Levels in plasma may be measured, for example, by HPLC.

The pharmaceutical compositions may be provided to the individual by a variety of routes such as subcutaneous, topical, oral and intramuscular. Administration of pharmaceutical compositions is accomplished orally or parenterally. Methods of parenteral delivery include topical, intra-arterial (directly to the tissue), intramuscular, subcutaneous, intramedullary, intrathecal, intraventricular, intravenous, intraperitoneal, or intranasal administration. The present invention also has the objective of providing suitable topical, oral, systemic and parenteral pharmaceutical formulations for use in the novel methods of treatment of the present invention. The compositions containing compounds identified according to this invention as the active ingredient for use in the modulation of signal-transduction kinase can be administered in a wide variety of therapeutic dosage forms in conventional vehicles for administration. For example, the compounds can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts.

For injection, the agents of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, capsules, or solutions. The pharmaceutical compositions of the present invention may be manufactured in a manner that is itself known, e.g., by means of conventional mixing, dissolving, granulating, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Kits

The present invention further provides kits for detecting MAPKAP-2 kinases and its attendant kinase activity. Such kits may be designed for detecting the level of a MAPKAP-2 kinase or polynucleotide, or may detect phosphorylation of its native or artificial substrate, e.g., Hsp-27 in a direct kinase assay or a coupled kinase assay, in which the level of phosphorylation and/or the kinase activity of MAPKAP-2 may be determined.

MAPKAP-2 kinase(s) and its attending kinase activity may be detected in any of a variety of samples, such as eukaryotic cells, bacteria, viruses, extracts prepared from such organisms and fluids found within living organisms. In general, the kits of the present invention comprise one or more containers enclosing elements, such as reagents or buffers, to be used in the assay.

A kit for detecting the level of MAPKAP-2 kinase or polynucleotide typically contains a reagent that binds to at least one member of the MAPK family and/or MAPKAP-2 kinase, DNA or RNA. To detect nucleic acid encoding a MAPKAP-2 kinase, the reagent may be a nucleic acid probe or a PCR primer. In a preferred embodiment, the kit further comprises other containers comprising one or more of the following: wash reagents and reagents capable of detecting the presence of bound nucleic acid probe. Examples of detection reagents include, but are not limited to radiolabeled probes, enzymatic labeled probes (horse radish peroxidase, alkaline phosphatase), and affinity labeled probes (biotin, avidin, or streptavidin).

To detect a MAPKAP-2 kinase, the reagent is typically an antibody. A kit useful for detecting the presence of MAPKAP-2 kinase in a sample comprises at least one container means having disposed therein the above-described reagent. The kit also contains a reporter group suitable for direct or indirect detection of the reagent (i.e., the reporter group may be covalently bound to the reagent or may be bound to a second molecule, such as Protein A, Protein G, immunoglobulin or lectin, which is itself capable of binding to the reagent). Suitable reporter groups include, but are not limited to, enzymes (e.g., horseradish peroxidase), substrates, cofactors, inhibitors, dyes, radionuclides, luminescent groups, fluorescent groups and biotin. Such reporter groups may be used to directly or indirectly detect binding of the reagent to a sample component using standard methods known to those of ordinary skill in the art.

In detail, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris-buffers, and the like), and containers which contain the reagents used to detect the hybridized probe, bound antibody, amplified product, or the like. Kits containing antibodies to MAPKAP-2, preferably monospecific antibodies such as monoclonal antibodies, or compositions of the NBP may also be provided, usually in lyophilized form in a container, either segregated or in conjunction with additional reagents, such as anti-antibodies, labels, etc.

A kit for detecting MAPKAK-2 kinase activity based on measuring the phosphorylation of Hsp-27 generally comprises Hsp-27 or its equivalent in combination with a suitable buffer. A kit for detecting MAPKAP-2 kinase activity based on detecting Hsp-27 activity generally comprises MAPKAP-2 in combination with a suitable MAPKAP-2 substrate, such as Hsp-27. Optionally, the kit may additionally comprise a suitable buffer and/or material for purification of MAPKAP-2 activation and before combination with substrate. Such kits may be employed in direct or coupled kinase assays, which may be performed as described above.

Thus, in another aspect, the present invention relates to a screening kit for identifying agonists, antagonists, ligands, binding partners, substrates, enzymes, etc. for the invention polypeptide; or compounds which decrease or enhance the production of such a polypeptide individually or as a complex, which comprises:

(a) a polypeptide of the present invention;

(b) a recombinant cell expressing the invention polypeptide;

(c) a cell membrane expressing the invention polypeptide; or

(d) an antibody to the invention polypeptide; wherein the invention polypeptide is that of SEQ ID NO:2.

It will be appreciated that in any such kit, (a), (b), (c) or (d) may comprise a substantial component.

One skilled in the art will readily recognize that the nucleic acid probes described in the present invention can readily be incorporated into one of the established kit formats which are well known in the art.

This invention is further illustrated by the following examples which should not be construed as limiting. The contents of all references, patents and published patent applications cited throughout this application are incorporated herein by reference.

### EXAMPLE I

I. Cloning of Invention cDNA Polynucleotide Sequences

(i) Full length MAPKAP-2 encoding cDNA sequence (SEQ ID NO: 3): Full length MAPKAP-2 cDNA spanning nucleotides 1-1200 was generated by ligation of three EST clones. These EST clones were: 343563 (gb:w69432); 610307 (gb:aa171519); and AI478890. A DNA piece representing the combined 342563 and 610307 pieces was sub-cloned into pThioHis vector and PCR amplified using the following oligonucleotides: 5′ ATA GTT TTA GCGGCCGC TCA GTG GGC CAG AGC CGC 3′ (SEQ ID NO: 5); and 5′ GCT CCA GAA GTG CTG GGT CCA GAC 3′ (SEQ ID NO: 6). PCR conditions were those recommended by the Clontech Advantage®-GC 2 PCR kit utilizing 10% (0.5 M) GC Melt buffer and Platinum Hi Fi DNA polymerase (Gibco BRL). This DNA fragment contained a 3′ NOT I restriction site for sub-cloning (fragment 1). The AI478890 EST DNA piece was PCR amplified utilizing the same conditions described above utilizing the following two oligonucleotides: 5′ GTG GGA TTC CC ATG TTG TCA AAC TCC CAG GGC 3′ (SEQ ID NO: 7); and 5′ TGG AGT AGA AGG GGG GAT ACC CAC 3′ (SEQ ID NO: 8). This fragment contained a 5′ BAM HI site for sub-cloning (fragment 2). The individual EST fragments were ligated into the TOPO TA vector using the rapid ligation kit method (Roche). Clones were miniprep purified using a Qiaspin Miniprep protocol (Qiagen). DNA fragments were isolated from the TA vector by restriction digest with AFL III and NOT I for fragment 1, and AFL III and BAM HI for fragment 2. Restricted inserts were gel purified using a Qiaspin Miniprep protocol (Qiagen). The inserts were ligated into pGEX 5X-1 (Pharmacia) that had been restricted with BAM HI and NOT I using the Roche rapid ligation method. Chemically competent E. coli 1-shot TOPO 10 cells were transformed and the resulting clones were miniprep purified as described above. Clones were checked by restriction digest to confirm release of a 1200 bp MAPKAP-2 DNA fragment and further confirmed by automated DNA sequencing on the ABI 393 XL with Taq FS Big Dye terminator Kit (Applied Biosystems). DNA sequencing of the above referenced 1200 bp MAPKAP-2 fragment is outlined in SEQ ID NO. 3 and its deduced amino acid sequence is outlined in SEQ 4. The cDNA sequence corresponding to the full length MAPKAP-2 encoding gene differs from the prior art MAPKAP-2 gene in at least the following respects:

DNA sequence comparison between the full length clone (SEQ ID NO: 3) and the prior art clone (clone with accession number X75346) revealed the following amino acid differences encoded by the respective DNA sequences: D117H; L247W; and V248S. Alignment of nine EST DNA fragments for MAPKAP-2 showed that these changes were conserved in all ESTs as well as other species such as mouse, hamster, rabbit, and drosophila.

The full length clone of the invention encoded 4 additional amino acids at the N terminus that were missing from the prior art clone.

(ii) Truncated MAPKAP-2 encoding cDNA sequence (SEQ ID NO:1): Truncated MAPKAP2 was PCR amplified using the full length MAPKAP2 cDNA described above as template and the following two oligonucleotides: 5′ CAGTCGGATCCCCTCCCAGGGCCAGAGCCCGC 3′ (SEQ ID NO: 9) and 5′ TAGCGCGGCCGCTCAGTGGGCCAGAGCCGCAGCCT 3′ (SEQ ID NO:10). PCR conditions were those recommended by the Clontech Advantage®-GC 2 PCR kit utilizing 10% (0.5 M) GC Melt buffer and Platinum Hi Fi DNA polymerase (Gibco BRL). The insert was ligated into pGEX 5X-1 (Pharmacia) that had been restricted with BAM HI and NOT I using the Roche rapid ligation method. Chemically competent E. coli 1-shot TOPO 10 cells were transformed and the resulting clones were miniprep purified as described above. Clones were checked by restriction digest to confirm release of a 1188 bp truncated MAPKAP-2 DNA fragment and further confirmed by automated DNA sequencing on the ABI 393 XL with Taq FS Big Dye terminator Kit (Applied Biosystems).

DNA sequence comparison between the truncated MAPKAP_{—}2 encoding clone (SEQ ID NO:1) and the prior art clone (clone with accession number X75346) revealed the following amino acid differences encoded by the respective DNA sequences: Dl 17H; L247W; and V248S. Alignment of nine EST DNA fragments for MAPKAP-2 showed that these changes were conserved in all ESTs as well as other species such as mouse, hamster, rabbit, and drosophila.

Expression of Full Length MAPKAP2 Clone in Sf-9/Baculovirus

MAPKAP-2 was sub-cloned in to pAcG3X (BD-Pharmingen) and expressed as a GST-fusion in Sf-9/baculovirus under the following conditions: 400 mL of 1.5×10⁶ Sf-9 cells/mL were infected at an moi=1 by adding 4.4 mL of a MAPKAP2 viral stock (titer 8.5×10⁷ pfu/mL). Cells were grown for 48 hours at 26° C. and activated by heat shock at 42° C. for 30 minutes in the presence of 2 mM Na₃VO₄, and 50 ng/mL Okadaic Acid. Cells were harvested by centrifugation at 1500 rpm for 10 minutes. Cell pellets were frozen at −20° C. and lysed in 100 mM Tris-HCL pH 8.0, 150 mM NaCl, 2 mM Na₃VO₄, 50 ng/mL Okadaic Acid, and Sigma Protease cocktail. The protein was purified by binding the supernatant to Glutathione Sepharose 4B (Pharmacia). The column was washed with 5 column volumes of column buffer and eluted with column buffer plus 20 mM Glutathione (Sigma). Column buffer for purification and elution 50 mM Tris pH 8.0, 0.5M NaCl, 1 mM DTT, 10% glycerol.

Expression of Truncated MAPKAP2 Clone in Sf-9/Baculovirus

Truncated MAPKAP-2 was sub-cloned in to pAcG3X (BD-Pharmingen) and expressed as a GST-fusion in Sf-9/baculovirus under the following conditions: 400 mL of 1.5×10⁶ Sf-9 cells/mL were infected at an moi=1 by adding 2.6 mL of a truncated MAPKAP2 viral stock (titer 1.5×10⁸ pfu/mL). Cells were grown for 48 hours at 26° C. and activated by heat shock at 42° C. for 30 minutes in the presence of 2 mM Na₃VO₄, and 50 ng/mL Okadaic Acid. Cells were harvested by centrifugation at 1500 rpm for 10 minutes. Cell pellets were frozen at −20° C. and lysed in 100 mM Tris-HCL pH 8.0, 150 mM NaCl, 2 mM Na₃VO₄, 50 ng/mL Okadaic Acid, and Sigma Protease cocktail. The protein was purified by binding the supernatant to Glutathione Sepharose 4B (Pharmacia). The column was washed with 5 column volumes of column buffer and eluted with column buffer plus 20 mM Glutathione (Sigma). Column buffer for purification and elution 50 mM Tris pH 8.0, 0.5M NaCl, 1 mM DTI, 10% glycerol.

### EXAMPLE 2

Mammalian Cell Expression

The polypeptides of the invention can also be expressed in either human embryonic kidney 293 (HEK293) cells or adherent dhfr CHO cells. To maximize protein expression, typically all 5′ and 3′ untranslated regions (UTRs) are removed from the protein cDNA prior to insertion into a pCDN or pCDNA3 vector. The cells can there after be transfected with individual receptor cDNAs by lipofectin and selected in the presence of appropriate amounts of (ca 400 mg/l ml) G418.

After a suitable period of time, i.e., about 3 weeks of selection, individual clones are picked and expanded for further analysis. HEK293 or CHO cells transfected with the vector alone serve as negative controls. To isolate cell lines stably expressing the individual receptors, about 24-36 clones are typically selected and analyzed by Northern blot analysis. Receptor mRNAs are generally detectable in about 50% of the G418 resistant clones analyzed.

### EXAMPLE 3

Functional Assay in Xenopus Oocytes

Capped RNA transcripts from linearized plasmid templates encoding the MAPKAP-2 cDNAs of the invention may be synthesized in vitro with RNA polymerases in accordance with standard procedures. In vitro transcripts are suspended in water at a final concentration of 0.2 mg/ml. Ovarian lobes can be removed from adult female toads, stage V defolliculated oocytes are obtained, and RNA transcripts (10 ng/oocyte) are injected in a 50 nl bolus using a microinjection apparatus.

Thereafter, two electrode voltage clamps are used to measure the currents from individual Xenopus oocytes in response to agonist exposure. Recordings are made in Ca²⁺ free Barth's medium at room temperature. The Xenopus system can be used to screen known ligands and tissue/cell extracts for activating ligands.

### EXAMPLE 4

Microphysiometric Assays

Activation of a wide variety of secondary messenger systems results in extrusion of small amounts of acid from a cell. The acid formed is largely as a result of the increased metabolic activity required to fuel the intracellular signaling process. The pH changes in the media surrounding the cell are very small but are detectable by the CYTOSENSOR microphysiometer (Molecular Devices Ltd., Menlo Park, Calif.). The CYTOSENSOR is thus capable of detecting the activation of a receptor which is coupled to an energy utilizing intracellular signaling pathway such as the G-protein coupled receptor of the present invention.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

Summary of Sequences

SEQ. ID. NO: 1 is the nucleotide sequence encoding the novel truncated MAPKAP-2 kinase of the invention, comprising 1191 nucleotide bases.

SEQ ID NO: 2 is the deduced amino acid sequence of the gene product of SEQ ID NO:1.

SEQ ID NO: 3 is the nucleotide sequence encoding the novel full length human MAPKAP-2 kinase of the invention, comprising 1200 nucleotide bases.

SEQ ID NO: 4 is the amino acid sequence of the gene product of SEQ ID NO: 3.

What is claimed:

1. An isolated nucleic acid molecule, comprising a sequence of nucleotides that encodes a human mitogen-activated protein kinase activating protein kinase-2 (MAPKAP-2 kinase), wherein the sequence of nucleotides is selected from the group consisting of:
a) a sequence of nucleotides that encodes a human MAPKAP-2 kinase and comprises the sequence of nucleotides set forth in SEQ ID NO:1;
b) a sequence of nucleotides that encodes a human MAPKAP-2 kinase and that hybridizes under conditions of high stringency to the complement of the sequence of nucleotides set forth in SEQ ID NO:1; and, if it is DNA, is fully complementary or, if it is RNA, is identical to mRNA native to a human cell;
c) a sequence of nucleotides degenerate with the human MAPKAP-2 polypeptide encoding sequence of (a) or (b); and
d) a sequence of nucleotides that encode an amino acid sequence as set forth in SEQ ID NO:2 or the corresponding fragment thereof.

2. An isolated nucleic acid molecule, comprising a coding region that encodes a splice variant of a MAPKAP-2 kinase, wherein the MAPKAP-2 kinase is encoded by a sequence of nucleotides as set forth in SEQ ID NO:1.

3. The isolated nucleic acid molecule according to claim 1, wherein the isolated nucleic acid molecule is cDNA.

4. The isolated nucleic acid molecule of claim 1, wherein said nucleic acid molecule comprises the sequence of nucleotides as set forth in SEQ ID NO:1.

5. The isolated nucleic acid molecule of claim 1, wherein said nucleic acid molecule comprises a sequence of nucleotides that hybridizes under stringent wash conditions to the complement of the sequence of nucleotides set forth in SEQ ID NO:1.

6. An isolated nucleic acid molecule that encodes a MAPKAP-2 kinase having an amino acid sequence as set forth in SEQ ID NO:2.

7. A substantially pure human MAPKAP-2 kinase encoded by the nucleic acid molecule of claim 1.

8. A substantially pure human MAPKAP-2 kinase encoded by a nucleotide sequence that is a splice variant of a isolated nucleic acid molecule that encodes a MAPKAP-2 kinase comprising the amino acid sequence set forth in SEQ ID NO:2.

9. A substantially pure human MAPKAP-2 kinase encoded by a nucleotide sequence as set forth in SEQ ID NO:1.

10. A substantially pure human MAPKAP-2 kinase encoded by a nucleic acid molecule comprising a sequence of nucleotides that hybridizes under stringent wash conditions to the complement of the sequence of nucleotides set forth in SEQ ID NO:1.

11. A substantially pure human MAPKAP-2 kinase comprising a sequence of amino acids as set forth in SEQ ID NO: 2.

12. Suitable host cells transfected or transformed with the nucleic acid molecule of claim 1, wherein the cells are bacterial cells, mammalian cells or amphibian oocytes, and the nucleic acid molecule is heterologous to the cells.

13. A method for detecting MAPKAP-2 messenger RNA in a biological sample comprising the steps of:
a) introducing the nucleic acid molecule of claim 1 into a suitable host cell that is suspected of expressing a MAPKAP-2 kinase under conditions favoring formation of a complex therebetween; and
b) detecting presence of said complex as indicative of presence of said MAPKAP-2 kinase in said sample.

14. A method for identifying DNA sequences encoding a MAPKAP-2 kinase, the method comprising probing a cDNA library or a genomic library with a labeled probe, and recovering from the library those sequences having a significant degree of homology relative to the probe, wherein the probe comprises the nucleotide sequence of claim 1.

15. A method for identifying MAPKAP2 kinase in a sample, comprising:
a) introducing the nucleic acid molecule of claim 1 into eukaryotic cells; and
b) detecting second messenger activity in the cells of step (a), wherein the activity is mediated by a polypeptide encoded by the introduced nucleic acid molecule.

16. A bioassay for identifying a test compound, which modulates the activity of a human MAPKAP-2 kinase, the bioassay comprising:
a) measuring the second messenger activity of eukaryotic cells transformed with DNA encoding a MAPKAP-2 kinase in the absence of the test compound, thereby obtaining a first measurement;
b) measuring the second messenger activity of eukaryotic cells transformed with DNA encoding the MAPKAP-2 kinase in the presence of the test compound, thereby obtaining a second measurement; and
c) comparing the first and second measurement and identifying those compounds that result in a difference between the first measurement and the second measurement as a test compound that modulates the activity of the MAPKAP-2 kinase, wherein the eukaryotic cells express a functional human parathyroid hormone-2 polypeptide.

17. A method for monitoring the effectiveness of treatment with a test compound for a MAPKAP-2 mediated disease state comprising the steps of
(i) obtaining a pre-administration sample from a subject suspected of having a dysfunctional MAPKAP-2 mediated disease state prior to administration of the test compound;
(ii) detecting a level of expression or activity of a MAPKAP-2 kinase encoding mRNA or genomic DNA in the pre-administration sample to obtain a first measurement;
(iii) detecting a level of expression or activity of a MAPKAp-2 kinase encoding mRNA or genomic DNA in a post-administration sample to obtain a second measurement;
(iv) comparing the level of expression or activity of the MAPKAP-2 kinase in the first and second measurement; and
(v) altering the administration of the compound to the subject accordingly.

18. A method for determining regression, progression or onset of a disease state manifested by a dysfunctional signal transducing MAPKAP-2 kinase, the method comprising the steps of (i) contacting a cDNA or mRNA containing sample from a subject suspected of suffering from said disease state with a nucleic acid hybridization probe comprising a sequence of nucleotides as set forth in SEQ ID NO: 1 under conditions favoring binding of the hybridization probe to the cDNA or mRNA to form a complex therebetween, and (ii) detecting said complex as an indication that said subject is at risk of developing said disease state.

19. A method for determining regression, progression or onset of a pathological disorder characterized by a dysfunctional signal transducing MAPKAP-2 kinase comprising: contacting a sample from a patient with the disorder with a detectable probe that is specific for the gene product of the isolated nucleic acid molecule having a sequence of nucleotides as set forth in SEQ ID NO:1 under conditions favoring formation of a probe/gene product complex, the presence of which is indicative of the regression, progression or onset of the pathological disorder in the patient.

20. The method of claim 19, wherein the probe is an antibody.

21. The method of claim 20, wherein the antibody is labeled with a radioactive label or an enzyme.

22. A method of screening test compounds for use as inflammation inhibitors, comprising the steps of:
a) contacting a test compound with a MAPKAP-2 kinase encoded by a polynucleotide comprising a nucleotide sequence selected from the group consisting of SEQ ID NO:1 and a sequence of nucleotides that encodes a human MAPKAP-2 kinase that hybridizes under conditions of high stringency to the complement of the sequence of nucleotides set forth in SEQ ID NO:1; and
b) testing the contacted MAPKAP-2 kinase protein for its ability to bind to or phosphorylate Hsp-27, wherein a test compound which inhibits phosphorylation of Hsp-27 by the MAPKAP-2 kinase or which inhibits the binding of the MAPKAP-2 kinase protein to the Hsp-27 is a candidate drug for treatment of inflammation.

23. A pharmaceutical composition comprising the polypeptide according to claim 7 in combination with a pharmaceutically acceptable carrier, diluent or excipient.

24. A method for monitoring the efficacy of an agent in correcting an abnormal level of the polypeptide of claim 7 in a subject prone thereto, comprising administering an effective amount of the agent to the subject and determining a level of the polypeptide in the subject following its administration, wherein a change in the level of the polypeptide towards a normal level is indicative of the efficacy of the agent.

25. An isolated nucleic acid molecule comprising a nucleotide sequence encoding a polypeptide that has at least 80% identity to the amino acid sequence set forth SEQ ID NO:2 over the entire length of SEQ ID NO:2, wherein 80% identity defines the amino acid alterations allowed for SEQ ID NO:2 which are determined by the equation and is calculated by the formula
Nₐ=Xₐ−(Xₐ Y),
wherein Nₐ is the maximum number of amino acid alterations, Xₐ is the total number of amino acids in SEQ ID NO:2, and Y has a value of 0.80, wherein any non-integer product of Xₐ and Y is rounded down to the nearest integer prior to subtracting such product from Xₐ.

26. A method for identifying ligand(s) that activate a MAPKAP-2 kinase, the method comprising:
(i) contacting endogenous-MAPKAP-2 kinase-deficient host cells with a candidate compound suspected of activating MAPKAP-2 kinase activity wherein the host cells contain a reporter gene functionally linked to a transcriptional control element, and an exogenous gene encoding the MAPKAP-2 kinase, wherein the transcriptional control element, upon activation, induces expression of the reporter gene(s);
(ii) monitoring induction of the reporter gene(s); and
(iii) identifying ligand(s) that activate the polypeptide.

27. An antibody that is specific for the gene product of the nucleic acid molecule of claim 1.

28. A recombinant non-human cell line which has been engineered to express a heterologous protein, the cell line comprising a host cell transformed or transfected with a heterologous nucleic acid molecule of claim 1 that inducibly expresses a MAPKAP-2 kinase of SEQ ID NO: 2.

29. An expression vector comprising the nucleic acid molecule of claim 1, operably linked to a regulatory nucleotide sequence that controls expression of the nucleic acid molecule in a host cell.

30. A method for detecting a binding partner for a MAPKAP-2 kinase in a sample suspected of containing the binding partner, comprising:
(i) contacting the sample with the MAPKAP-2 of SEQ ID NO:2 under conditions favoring binding of the MAPKAp-2 kinase to the binding partner;
(ii) determining presence of the binding partner in the sample by detecting binding of the MAPKAP-2 kinase to the binding partner.

31. A method for identifying a compound which modulates the binding or kinase activity of a kinase polypeptide consisting of the amino acid sequence of SEQ ID NO:2; the method comprising:
a) contacting a cell expressing the polypeptide with a test compound under conditions suitable for modulation of the binding or kinase activity of the polypeptide; and
b) detecting modulation of the activity of the binding or kinase polypeptide by the test compound.

32. The method according to claim 31, wherein said agent inhibits MAPKAP-2 activity.

33. The method according to claim 31, wherein said agent stimulates MAPKAP-2 activity.

34. The method according to claim 31, wherein the agent is an antibody that specifically binds to the MAPKAP-2 kinase.

35. The method according to claim 31, wherein the agent modulates expression of MAPKAP-2 by modulating transcription of a MAPKAP-2 gene or translation of a MAPKAP-2 mRNA.

36. The method according to claim 31, wherein the agent is a nucleic acid molecule having a nucleotide sequence that is antisense to the coding strand of a MAPKAP-2 mRNA or a MAPKAP-2 gene.

37. A method for modulating endogenous signal transducing activity of a MAPKAP-2 kinase in a mammal comprising contacting a cell capable of expressing MAPKAP-2 with the compound of claim 31.

38. The method of claim 37, wherein said modulation of the activity of the polypeptide is detected by direct binding of the test compound to the polypeptide.

39. The method of claim 38, wherein said direct binding is determined by lysing the cell and performing an immunoprecipitation.

40. The method of claim 39, wherein said direct binding is determined by a yeast two-hybrid assay.

41. The method of claim 40, wherein said modulation of the activity of the polypeptide is detected by use of an assay for MAPKAP-2 kinase activity.

42. The method of claim 41, wherein said assay for MAPKAP-2 kinase activity is based on the phosphorylation of a MAPKAP-2 substrate.

43. A method for identifying a compound which modulates the binding or kinase activity of a naturally occurring allelic variant of a polypeptide consisting of the amino acid sequence of SEQ ID NO:2, wherein the allelic variant is encoded by a nucleic acid molecule which hybridizes to the complement of a nucleic acid molecule consisting of SEQ ID NO:1 in 6×SSC at 45° C., followed by one or more washes in 0.2.times.SSC, 0.1% SDS at 50-65° C., the method comprising:
a) contacting a cell expressing the allelic variant with a test compound under conditions suitable for modulation of the binding or kinase activity of the allelic variant; and
b) detecting modulation of the binding or kinase activity of the allelic variant by the test compound.

44. Isolated polypeptide comprising an amino acid sequence provided in SEQ ID NO:2, or a variant thereof that is at least 80% identical to SEQ ID NO:2 and that differs from SEQ ID NO:2 only in one or more amino acid substitutions, additions of terminal amino acid residues and/or deletions of terminal amino acid residues, wherein the ability of the variant to phosphorylate Hsp-27 is not substantially diminished.

45. A method of treating a subject having a disorder characterized by aberrant MAPKAP-2 kinase or nucleic acid expression or activity comprising administering an agent which is a MAPKAP-2 modulator to the subject.

46. The method according to claim 45 wherein the MAPKAP-2 modulator is a MAPKAP-2 kinase.

47. The method according to claim 45, wherein the MAPKAP-2 modulator is a MAPKAP-2 nucleic acid molecule.

48. The method according to claim 45, wherein the MAPKAP-2 modulator is a peptide, peptidomimetic, or other small molecule.

49. The method according to claim 45, wherein the disorder characterized by aberrant MAPKAP-2 protein or nucleic acid expression is an immune related disorder.

50. A method of phosphorylating a serine-containing substrate which comprises:
a) incubating the substrate with an effective concentration of ATP and an enzyme having at least 84% homology to SEQ ID NO:1, wherein said enzyme phosphorylates Hsp-27; and
b) measuring the amount of phosphorylation of the substrate.

51. The method of claim 50, which further comprises the steps of forming a mixture of the enzyme and a candidate antagonist or a candidate agonist of the enzyme and measuring the effect of said candidate antagonist or candidate agonist on the amount of phosphorylation on the substrate.

52. A method for identifying a reagent that modulates mitogen-activating protein kinase (MAPKAP-2) activity, the method comprising:
a) obtaining a test sample containing an MAPKAP-2 kinase characterized as having serine, threonine, and tyrosine kinase activity, and a reagent;
b) incubating the test sample with an MAPKAP-2 substrate and with labeled phosphate under conditions sufficient to allow phosphorylation of the substrate;
c) determining the rate of incorporation of labeled phosphate into the substrate, wherein the rate of incorporation is a measure of MAPKAP-2 activity; and
d) comparing the effect of the reagent on MAPKAP-2 activity relative to a control, wherein a change in MAPKAP-2 activity indicates the presence of a reagent able to modulate MAPKAP-2 activity.

53. The method of claim 51, wherein said MAPKAP-2 substrate is one or more of Hsp-25, Hsp-27 and ALT2.

54. The method of claim 51, wherein said modulation is inhibition of MAPKAP-2 activity.

55. The method of claim 51, wherein the reagent is selected from the group consisting of an antisense oligonucleotide and a ribozyme.

56. The method of claim 51, wherein the reagent is selected from the group consisting of a tumor necrosis factor and an interleukin-1.

57. A method for identifying a reagent that modulates MAPKAP-2 synthesis, the method comprising:
a) providing a test sample containing an MAPKAP-2 kinase having serine, threonine, and tyrosine kinase activity;
b) incubating the test sample in the presence of a reagent;
c) fractionating proteins present in the sample by gel electrophoresis;
d) transferring the proteins onto a membrane;
e) probing the proteins with a labeled antibody specific to an MAPKAP-2 kinase, wherein the level of MAPKAP-2 synthesis is determined by the amount of antibody detected; and
f) comparing the effect of the reagent on MAPKAP-2 synthesis relative to a control, wherein a change in MAPKAP-2 synthesis indicates the presence of a reagent able to modulate MAPKAP-2 synthesis.

58. A method for identifying a reagent that modulates MAPKAP-2 expression, the method comprising:
a) providing a test sample in which an MAPKAP-2 polynucleotide is expressed;
b) incubating the test sample in the presence of a reagent;
c) isolating polyadenylated RNA from the test sample;
d) incubating the polyadenylated RNA with a polynucleotide probe specific for a MAPKAP-2 kinase;
e) determining the amount of the probe hybridized to the polyadenylated RNA, wherein the level of expression of MAPKAP-2 is directly related to the amount of MAPKAP-2 probe hybridized to the RNA; and
f) comparing the effect of the reagent on MAPKAP-2 expression relative to a control, wherein a change in MAPKAP-2 expression indicates the presence of a reagent able to modulate MAPKAP-2 expression.

59. The method of claim 58, wherein the reagent is selected from the group consisting of a polynucleotide, a polypeptide, and an antibody.

60. The method of claim 58, wherein the reagent is selected from the group consisting of an antisense oligonucleotide and a ribozyme.

61. A kit useful for the detection of mitogen-activating protein kinase activating protein kinase 2 (MAPKAP-2), said kit comprising a buffer and a labeled antibody which specifically binds to a MAPKAP-2 kinase having serine, threonine, and tyrosine kinase activity, wherein the sample to be tested is mixed with the buffer and the antibody.

62. A kit useful for the detection of mitogen-activating protein kinase activating protein kinase 2 (MAPKAP-2) encoding nucleic acid, said kit comprising a buffer and nucleic acid molecule comprising at least about 20 nucleotides capable of hybridizing to a nucleic acid sequence encoding MAPKAP-2 or a complement thereof under stringent hybridization conditions, and instructions for use thereof.

63. An isolated nucleic acid molecule, comprising a sequence of nucleotides that encodes a human mitogen-activated protein kinase activating protein kinase-2 (MAPKAP-2 kinase), wherein the sequence of nucleotides is selected from the group consisting of:
(a) a sequence of nucleotides that encodes a human MAPKAP-2 kinase and comprises the sequence of nucleotides set forth in SEQ D NO:3;
(b) a sequence of nucleotides that encodes a human MAPKAP-2 kinase and that hybridizes under conditions of high stringency to the complement of the sequence of nucleotides set forth in SEQ ID NO:3; and, if it is DNA, is fully complementary or, if it is RNA, is identical to mRNA native to a human cell;
(c) a sequence of nucleotides degenerate with the human MAPKAP-2 polypeptide encoding sequence of (a) or (b);
(d) a sequence of nucleotides that encode an amino acid sequence as set forth in SEQ ID NO: 4 or the corresponding fragment thereof.

64. An isolated nucleic acid molecule, comprising a coding region that encodes a splice variant of a MAPKAP-2 kinase, wherein the MAPKAP-2 kinase is encoded by a sequence of nucleotides as set forth in SEQ ID NO: 3.

65. An isolated nucleic acid molecule that encodes a MAPKAP-2 kinase having an amino acid sequence as set forth in SEQ ID NO:4.

66. A substantially pure MAPKAP-2 kinase encoded by a nucleotide sequence that is a splice variant of a isolated nucleic acid molecule that encodes a MAPKAP-2 kinase comprising the amino acid sequence set forth in SEQ ID NO:4.

67. Suitable host cells transfected or transformed with the nucleic acid molecule of claim 62, wherein the cells are bacterial cells, mammalian cells or amphibian oocytes, and the nucleic acid molecule is heterologous to the cells.

68. An substantially pure MAPKAP-2 kinase encoded by the nucleic acid molecule of claim 63.

69. An isolated nucleic acid molecule comprising a nucleotide sequence having at least 80% identity to a nucleotide sequence encoding a polypeptide comprising the amino acid sequence set forth SEQ ID NO:2, which may include up to Nₐ nucleic acid alterations over the entire length of SEQ ID NO:1, wherein Nₐ represents the maximum number of such alterations and is calculated by the formula
Nₐ=Xₐ−(Xₐ Y),
In which Xₐ is the total number of nucleotides in SEQ D NO:1, and Y has a value of 0.80, wherein any non-integer product of Xₐ and Y is rounded down to the nearest integer prior to subtracting such product from Xₐ, wherein said polypeptide has MAPKAp-2 activity.


SEQUENCE LISTING (4 sequences)

SEQ ID NO: 1 - DNA, 1191 bp, Human
tcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgcccgcacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacatcctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggaggagatgaccagtgccttggccacaatgcgcgttgactacgagcagatcaagataaaaaagattgaagatgcatccaaccctctgctgctgaagaggcggaagaaagctcgggccctggaggctgcggctctggcccactga

SEQ ID NO: 2 - protein, 396 aa, Human
SQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKEEMTSALATMRVDYEQIKIKKIEDASNPLLLKRRKKARALEAAALAH

SEQ ID NO: 3 - DNA, 1203 bp, Human
atgctgtccaactcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgcccgcacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacatcctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggaggagatgaccagtgccttggccacaatgcgcgttgactacgagcagatcaagataaaaaagattgaagatgcatccaaccctctgctgctgaagaggcggaagaaagctcgggccctggaggctgcggctctggcccactga

SEQ ID NO: 4 - protein, 400 aa, Human
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKEEMTSALATMRVDYEQIKIKKIEDASNPLLLKRRKKARALEAAALAH